SEQUENCE LISTING (80 sequences)

SEQ ID NO: 1 - DNA, 51 bp, Cannabis sativa
  source 1..51: mol_type = genomic DNA
ctgaattcttcgtcttctagttcctgtacacttcacactcacggggcaact

SEQ ID NO: 2 - DNA, 51 bp, Cannabis sativa
  source 1..51: mol_type = genomic DNA
tcatcagtcctattagaatacgtttgcccactttcttccttttgggatcat

SEQ ID NO: 3 - DNA, 51 bp, Cannabis sativa
  source 1..51: mol_type = genomic DNA
aagaataggctttgtgttagttagaattagttactttgctctcacggttga

SEQ ID NO: 4 - DNA, 51 bp, Cannabis sativa
  source 1..51: mol_type = genomic DNA
agccaaacggttgccgccctcatatctattaattaaggcttctcaattagc

SEQ ID NO: 5 - DNA, 51 bp, Cannabis sativa
  source 1..51: mol_type = genomic DNA
ctatttaattatattctctatattaacagggcttgaagaggactacactct

SEQ ID NO: 6 - DNA, 51 bp, Cannabis sativa
  source 1..51: mol_type = genomic DNA
cgtcgtcgggtggttgactgcatttctaaactccgagctagattttcatag

SEQ ID NO: 7 - DNA, 51 bp, Cannabis sativa
  source 1..51: mol_type = genomic DNA
tctctgacacatgcaaggacgattccttgacttaaaaataggcaatggaag

SEQ ID NO: 8 - DNA, 51 bp, Cannabis sativa
  source 1..51: mol_type = genomic DNA
gattacatttcattttaggactcctgattgaattgaattgaataccctttt

SEQ ID NO: 9 - DNA, 51 bp, Cannabis sativa
  source 1..51: mol_type = genomic DNA
agtatagaaaatcgttaatccatttaatggccccttcccataaacttagcc

SEQ ID NO: 10 - DNA, 51 bp, Cannabis sativa
  source 1..51: mol_type = genomic DNA
gatccaatccaattcatgccatattggaaggatatagagtgcatatacatg

SEQ ID NO: 11 - DNA, 51 bp, Cannabis sativa
  source 1..51: mol_type = genomic DNA
taccagacagtgaagatgttgtgaagaaagcagaacccttgagtgacaaac

SEQ ID NO: 12 - DNA, 51 bp, Cannabis sativa
  source 1..51: mol_type = genomic DNA
cccaaccgcccaaagtaacacctgtcatattacaaaatcaattaattatta

SEQ ID NO: 13 - DNA, 51 bp, Cannabis sativa
  source 1..51: mol_type = genomic DNA
gaaaaagggaaagaacgtgatatagctagttgattggaggaataggtttgt

SEQ ID NO: 14 - DNA, 51 bp, Cannabis sativa
  source 1..51: mol_type = genomic DNA
ccagacaggtacatgcatatgtggtcattttaccatgtcaaaaacagtatt

SEQ ID NO: 15 - DNA, 51 bp, Cannabis sativa
  source 1..51: mol_type = genomic DNA
gatttctgactcagagcctttggtattaaaagttcaggatgtagcgtgatc

SEQ ID NO: 16 - DNA, 51 bp, Cannabis sativa
  source 1..51: mol_type = genomic DNA
cactaaaatgttgtacactaaatttgacatcattaatagtgttgaatactg

SEQ ID NO: 17 - DNA, 51 bp, Cannabis sativa
  source 1..51: mol_type = genomic DNA
aattaaaataatcatatattcattcccaacttcctttcttctttaactttc

SEQ ID NO: 18 - DNA, 51 bp, Cannabis sativa
  source 1..51: mol_type = genomic DNA
acaaaagataatatcggacatagaggatagaggtatggagccgaacctttg

SEQ ID NO: 19 - DNA, 51 bp, Cannabis sativa
  source 1..51: mol_type = genomic DNA
catatatctttttctttgtcatgctgttatgttatgtaaagaaaaattgaa

SEQ ID NO: 20 - DNA, 20 bp, synthetic construct
  source 1..20: mol_type = other DNA
aggaagacgaggaggaggac

SEQ ID NO: 21 - DNA, 20 bp, synthetic construct
  source 1..20: mol_type = other DNA
ggattccctgaggcattgta

SEQ ID NO: 22 - DNA, 20 bp, synthetic construct
  source 1..20: mol_type = other DNA
tctgtccaactttccccatc

SEQ ID NO: 23 - DNA, 20 bp, synthetic construct
  source 1..20: mol_type = other DNA
tgctctgattgcttccatga

SEQ ID NO: 24 - DNA, 20 bp, synthetic construct
  source 1..20: mol_type = other DNA
cgaccatcgagtgcttacaa

SEQ ID NO: 25 - DNA, 20 bp, synthetic construct
  source 1..20: mol_type = other DNA
agcacccaattcctgagctt

SEQ ID NO: 26 - DNA, 20 bp, synthetic construct
  source 1..20: mol_type = other DNA
agcccacctgtggtgttaag

SEQ ID NO: 27 - DNA, 20 bp, synthetic construct
  source 1..20: mol_type = other DNA
tggccagatttgtgttgtgt

SEQ ID NO: 28 - DNA, 20 bp, synthetic construct
  source 1..20: mol_type = other DNA
ggctggtttcgttgttgttt

SEQ ID NO: 29 - DNA, 20 bp, synthetic construct
  source 1..20: mol_type = other DNA
gaaatgggcatcaagaggaa

SEQ ID NO: 30 - DNA, 1883 bp, Cannabis sativa
  source 1..1883: mol_type = other DNA
ttggagaatgtctcgcacgggcagcttcaggctttatcggctgtgcctcctgattgccctagtttggatcaggataggtccgatgggggaaattcggcttatgtggtcactccgcctccgattatggaaggccgcggcgttgtgaagcgttttgggactcggtctcatgtcgttccaatgcattcggattggttttcacctgctacagtgcatagactagagagacaagtggtgccacattttttctcaggaaaatcaccagaccatactcctgagaaatacatggaatgtaggaactatgttgttgccaaatacatggagaatccagaaagacggttcactgtgtctgatttcgtgggagtgatacctggtcttgaaactgaagatttaagtcgaatagttcggtttcttgatcactggggaataatcaattattgtgctgctgtatcaagtcatgagccatggaatagcagttcctacttaaaggaggagtcaaatggtgagattcacgtgccatctgctgcgttaaagtccattgatagcttaatcaagtttgaaaagcccaaatgcaagctcaaggcagctgatgtttattcatccctttcaagtcatgacaacaatgtctctgatttggacaacagaattcgtgagcatttatctgaaaatcgttgcaatagttgttctcgatctctacccactgtatgctatcagtcacacaaggaggtggatataatgctatgctctgattgcttccatgatggaaggtatgtgattggtcactcaagcttagattttacgaggatggattcaacaaaagattatggcgatccagatggggaaagttggacagatcaagaaacgttgttgctacttgaggcagtggaaatgtacaatgagaactggaatgaaattgcagagcatgttggaactaagtctaaagcccaatgcattcttcatttccttcgcctgcctgtggaagatggccttctggaaaatattgaagttcctgggaatcctatatcatccagtatgtcaaatggagctgttcatggacaaataattgcaaatggggattcagcaggatcatgccaacaagatgctgattttgaaagcagattcccttttgccaattctgggaatccggtcatgtccctggttgcctttctggcttcttcggttggcccaagagtcgctgcagcatgtgcacatgcatccttggcagcgttgtcggaggaaagtagtttatctgcatcggagaacctgttacataaggaaggaccaggacagagtaataggatgaactcagagattattcatggtagagatggtggccctcagggagagattgcaaattcagctcaccataaagctgttgcttcaaggggtcaaaatgaaggggctactccattatcagcagaaaaggttaaagctgctgctaaagttggcttggctgctgcagcaacgaaggccaaattgtttgctgatcatgaagaacgagaaatccagagactctctgccaatattataaatcatcagctgaagcgattggagctgaagttaaagcaatttgctgaagtggaaacattcctaatgaaagaatgtgaacaagtggagaggacaagacagagacttttggctgagcgatctcgttttatggcagcacaaatgggagctagcgggatcacttctatgactctaccaggagcacctggtccttccatggttaacaactcaggtaacaatagacaacaacttatgtcagccgcaccttcacaaccgagcatatcaggatacaacaacaatcaccaaccacaacaggttcatccccacatgccattcaggcctcggcagcaaatgtttgctatggggccaaggatgccgct

SEQ ID NO: 31 - DNA, 2138 bp, Cannabis sativa
  source 1..2138: mol_type = other DNA
caggatcgtgaggatgattcagaggatggtgtccaacctaaccctcaatctggctcttccgcacccgatccctcttcttcctctcaagagagtgaggtctctcccgacggtgtggttcgttgctctgattttccacctgtcatgaggcgcatcttgaatcgccctcacccttccgttatggccattgttgctttggagcgagctaatgagtttggggaaagcaagggtcaaggtcagggaagctctttaatgtttttggagaatgtctcgcacgggcagcttcaggctttatcggctgtgcctcctgattgccctagtttggatcaggataggtccgatgggggaaattcggcttatgtggtcactccgcctccgattatggaaggccgcggcgttgtgaagcgttttgggactcggtctcatgtcgttccaatgcattcggattggttttcacctgctacagtgcatagactagagagacaagtggtgccacattttttctcaggaaaatcaccagaccatactcctgagaaatacatggaatgtaggaactatgttgttgccaaatacatggagaatccagaaagacggttcactgtgtctgatttcgtgggagtgatacctggtcttgaaactgaagatttaagtcgaatagttcggtttcttgatcactggggaataatcaattattgtgctgctgtatcaagtcatgagccatggaatagcagttcctacttaaaggaggagtcaaatggtgagattcacgtgccatctgctgcgttaaagtccattgatagcttaatcaagtttgaaaagcccaaatgcaagctcaaggcagctgatgtttattcatccctttcaagtcatgacaacaatgtctctgatttggacaacagaattcgtgagcatttatctgaaaatcgttgcaatagttgttctcgatctctacccactgtatgctatcagtcacacaaggaggtggatataatgctatgctctgattgcttccatgatggaaggtatgtgattggtcactcaagcttagattttacgaggatggattcaacaaaagattatggcgatccagatggggaaagttggacagatcaagaaacgttgttgctacttgaggcagtggaaatgtacaatgagaactggaatgaaattgcagagcatgttggaactaagtctaaagcccaatgcattcttcatttccttcgcctgcctgtggaagatggccttctggaaaatattgaagttcctgggaatcctatatcatccagtatgtcaaatggagctgttcatggacaaataattgcaaatggggattcagcaggatcatgccaacaagatgctgattttgaaagcagattcccttttgccaattctgggaatccggtcatgtccctggttgcctttctggcttcttcggttggcccaagagtcgctgcagcatgtgcacatgcatccttggcagcgttgtcggaggaaagtagtttatctgcatcggagaacctgttacataaggaaggaccaggacagagtaataggatgaactcagagattattcatggtagagatggtggccctcagggagagattgcaaattcagctcaccataaagctgttgcttcaaggggtcaaaatgaaggggctactccattatcagcagaaaaggttaaagctgctgctaaagttggcttggctgctgcagcaacgaaggccaaattgtttgctgatcatgaagaacgagaaatccagagactctctgccaatattataaatcatcagctgaagcgattggagctgaagttaaagcaatttgctgaagtggaaacattcctaatgaaagaatgtgaacaagtggagaggacaagacagagacttttggctgagcgatctcgttttatggcagcacaaatgggagctagcgggatcacttctatgactctaccaggagcacctggtccttccatggttaacaactcaggtaacaatagacaacaacttatgtcagccgcaccttcacaaccgagcatatcaggatacaacaacaatcaccaaccacaacaggttcatccccacatgccattcaggcctcggcagcaaatgtttgctatggggccaaggatgccgct

SEQ ID NO: 32 - DNA, 2150 bp, Cannabis sativa
  source 1..2150: mol_type = other DNA
caggatcgtgaggatgattcagaggatggtgtccaacctaaccctcaatctggctcttccgcacccgatccctcttcttcctctcaagagagtgaggtctctcccgacggtgtggttcgttgctctgattttccacctgtcatgaggcgcatcttgaatcgccctcacccttccgttatggccattgttgctttggagcgagctaatgagtttggggaaagcaagggtcaaggtcagggaagctctttaatgtttttggagaatgtctcgcacgggcagcttcaggctttatcggctgtgcctcctgattgccctagtttggatcaggataggtccgatgggggaaattcggcttatgtggtcactccgcctccgattatggaaggccgcggcgttgtgaagcgttttgggactcggtctcatgtcgttccaatgcattcggattggttttcacctgctacagtgcatagactagagagacaagtggtgccacattttttctcaggaaaatcaccagaccatactcctgagaaatacatggaatgtaggaactatgttgttgccaaatacatggagaatccagaaaaacggttcactgtgtctgatttcgtgggagtgatacctggtcttgaaactgaagatttaagccgaatagttcggtttcttgatcactggggaataatcaattattgtgctgctgtatcaagtcatgagccatggaatagcagttcctacttaaaggaggagtcaaatggtgagattcacgtgccatctgctgcattaaagtccattgatagcttaatcaagtttgaaaagcccaaatgcaagctcaaggcagctgatgtttattcatccctttcaagtcatgacaacaatgtctctgatttggacaacagaattcgtgagcatttatctgaaaatcgttgcaatagttgttctcgatctctacccactgtatgctatcagtcacacaaggaggtggatataatgctatgctctgattgcttccatgatggaaggtatgtgattggtcactcaagcttagattttatgaggatggattcaacaaaagattatggcgatccagatggggaaagttggacagatcaagaaacgttgttgctacttgaggcagtggaaatgtacaatgagaactggaatgaaattgcagagcatgttggaactaagtctaaagcccaatgcattcttcatttccttcgcctgcctgtggaagatggccttctggaaaatattgaagttcctgggaatcctatatcatccagtatgtcaaatggagctgttcatggacaaataattgcaaatggggattcagcaggatcatgccaacaagatgctgattttgaaagcagattcccttttgccaattctgggaatccggtcatgtccctggttgcctttctggcttcttcggttggcccaagagtcgctgcagcatgtgcacatgcatccttggcagcattgtcggaggaaagtagtttatctgcatcggagaacctgttacataaggaaggaccaggacagagtaataggatgaactcagagattattcatggtagagatggtggccctcagggagagattgcaaattcagctcaccataaagctgttgcttcaaggggtcaaaatgaaggggctactccattatcagcagaaaaggttaaagctgctgctaaagttggcttggctgctgcagcaacaaaggccaaattgtttgctgatcatgaagaacgagaaatccagagactctctgccaatattataaatcatcagctgaagcgattagagctgaagttaaagcaatttgctgaagtggaaacattcctaatgaaagawtgtgaacaagtggagaggacaagacagagacttttggctgagcgatctcgttttatggcagcacaaatgggagctagcgggatcacttctatgactctaccaggagcacctggtccttccatggttaacaactcaggtaacaatagacaacaacttatgtcagccgcaccttcacaaccgagcatatcaggatacaacaacaatcaccaaccacaacaggttcatccccacatgccattcaggcctcggcagcaaatgtttgctatggggccaaggatgccgcttggtgccataca

SEQ ID NO: 33 - DNA, 2403 bp, Cannabis sativa
  source 1..2403: mol_type = other DNA
atgccagcttcttctccttcagatgggcggggcaaatggaggaagcggcggagagaaccccaaatcaatcggaggctgaagcaggaagacgaggaggaggacgaagacgacgccgttccagatgagaatcttgaacaacaacaggatcgtgaggatgattcagaggatggtgtccaacctaaccctcaatctggctcttccgcacccgatccctcttcttcctctcaagagagtgaggtctctcccgacggtgtggttcgttgctctgattttccacctgtcatgaggcgcatcttgaatcgccctcacccttccgttatggccattgttgctttggagcgagctaatgagtttggggaaagcaagggtcaaggtcagggaagctctttaatgtttttggagaatgtctcgcacgggcagcttcaggctttatcggctgtgcctcctgattgccctagtttggatcaggataggtccgatgggggaaattcggcttatgtggtcactccgcctccgattatggaaggccgcggcgttgtgaagcgttttgggactcggtctcatgtcgttccaatgcattcggattggttttcacctgctacagtgcatagactagagagacaagtggtgccacattttttctcaggaaaatcaccagaccatactcctgagaaatacatggaatgtaggaactatgttgttgccaaatacatggagaatccagaaaaacggttcactgtgtctgatttcgtgggagtgatacctggtcttgaaactgaagatttaagccgaatagttcggtttcttgatcactggggaataatcaattattgtgctgctgtatcaagtcatgagccatggaatagcagttcctacttaaaggaggagtcaaatggtgagattcacgtgccatctgctgcattaaagtccattgatagcttaatcaagtttgaaaagcccaaatgcaagctcaaggcagctgatgtttattcatccctttcaagtcatgacaacaatgtctctgatttggacaacagaattcgtgagcatttatctgaaaatcgttgcaatagttgttctcgatctctacccactgtatgctatcagtcacacaaggaggtggatataatgctatgctctgattgcttccatgatggaaggtatgtgattggtcactcaagcttagattttatgaggatggattcaacaaaagattatggcgatccagatggggaaagttggacagatcaagaaacgttgttgctacttgaggcagtggaaatgtacaatgagaactggaatgaaattgcagagcatgttggaactaagtctaaagcccaatgcattcttcatttccttcgcctgcctgtggaagatggccttctggaaaatattgaagttcctgggaatcctatatcatccagtatgtcaaatggagctgttcatggacaaataattgcaaatggggattcagcaggatcatgccaacaagatgctgattttgaaagcagattcccttttgccaattctgggaatccggtcatgtccctggttgcctttctggcttcttcggttggcccaagagtcgctgcagcatgtgcacatgcatccttggcagcattgtcggaggaaagtagtttatctgcatcggagaacctgttacataaggaaggaccaggacagagtaataggatgaactcagagattattcatggtagagatggtggccctcagggagagattgcaaattcagctcaccataaagctgttgcttcaaggggtcaaaatgaaggggctactccattatcagcagaaaaggttaaagctgctgctaaagttggcttggctgctgcagcaacaaaggccaaattgtttgctgatcatgaagaacgagaaatccagagactctctgccaatattataaatcatcagctgaagcgattagagctgaagttaaagcaatttgctgaagtggaaacattcctaatgaaagaatgtgaacaagtggagaggacaagacagagacttttggctgagcgatctcgttttatggcagcacaaatgggagctagcgggatcacttctatgactctaccaggagcacctggtccttccatggttaacaactcaggtaacaatagacaacaacttatgtcagccgcaccttcacaaccgagcatatcaggatacaacaacaatcaccaaccacaacaggttcatccccacatgccattcaggcctcggcagcaaatgtttgctatggggccaaggatgccgcttggtgccatacagtcaccatcatctgctccaaatgtcatgtacaatgcctcagggaatccccaacctagtatgaatcatccgatgttaaggccagtacctggtactagctctggtttggggtga

SEQ ID NO: 34 - protein, 627 aa, Cannabis sativa
LENVSHGQLQALSAVPPDCPSLDQDRSDGGNSAYVVTPPPIMEGRGVVKRFGTRSHVVPMHSDWFSPATVHRLERQVVPHFFSGKSPDHTPEKYMECRNYVVAKYMENPERRFTVSDFVGVIPGLETEDLSRIVRFLDHWGIINYCAAVSSHEPWNSSSYLKEESNGEIHVPSAALKSIDSLIKFEKPKCKLKAADVYSSLSSHDNNVSDLDNRIREHLSENRCNSCSRSLPTVCYQSHKEVDIMLCSDCFHDGRYVIGHSSLDFTRMDSTKDYGDPDGESWTDQETLLLLEAVEMYNENWNEIAEHVGTKSKAQCILHFLRLPVEDGLLENIEVPGNPISSSMSNGAVHGQIIANGDSAGSCQQDADFESRFPFANSGNPVMSLVAFLASSVGPRVAAACAHASLAALSEESSLSASENLLHKEGPGQSNRMNSEIIHGRDGGPQGEIANSAHHKAVASRGQNEGATPLSAEKVKAAAKVGLAAAATKAKLFADHEEREIQRLSANIINHQLKRLELKLKQFAEVETFLMKECEQVERTRQRLLAERSRFMAAQMGASGITSMTLPGAPGPSMVNNSGNNRQQLMSAAPSQPSISGYNNNHQPQQVHPHMPFRPRQQMFAMGPRMP

SEQ ID NO: 35 - protein, 712 aa, Cannabis sativa
QDREDDSEDGVQPNPQSGSSAPDPSSSSQESEVSPDGVVRCSDFPPVMRRILNRPHPSVMAIVALERANEFGESKGQGQGSSLMFLENVSHGQLQALSAVPPDCPSLDQDRSDGGNSAYVVTPPPIMEGRGVVKRFGTRSHVVPMHSDWFSPATVHRLERQVVPHFFSGKSPDHTPEKYMECRNYVVAKYMENPERRFTVSDFVGVIPGLETEDLSRIVRFLDHWGIINYCAAVSSHEPWNSSSYLKEESNGEIHVPSAALKSIDSLIKFEKPKCKLKAADVYSSLSSHDNNVSDLDNRIREHLSENRCNSCSRSLPTVCYQSHKEVDIMLCSDCFHDGRYVIGHSSLDFTRMDSTKDYGDPDGESWTDQETLLLLEAVEMYNENWNEIAEHVGTKSKAQCILHFLRLPVEDGLLENIEVPGNPISSSMSNGAVHGQIIANGDSAGSCQQDADFESRFPFANSGNPVMSLVAFLASSVGPRVAAACAHASLAALSEESSLSASENLLHKEGPGQSNRMNSEIIHGRDGGPQGEIANSAHHKAVASRGQNEGATPLSAEKVKAAAKVGLAAAATKAKLFADHEEREIQRLSANIINHQLKRLELKLKQFAEVETFLMKECEQVERTRQRLLAERSRFMAAQMGASGITSMTLPGAPGPSMVNNSGNNRQQLMSAAPSQPSISGYNNNHQPQQVHPHMPFRPRQQMFAMGPRMP

SEQ ID NO: 36 - protein, 716 aa, Cannabis sativa
QDREDDSEDGVQPNPQSGSSAPDPSSSSQESEVSPDGVVRCSDFPPVMRRILNRPHPSVMAIVALERANEFGESKGQGQGSSLMFLENVSHGQLQALSAVPPDCPSLDQDRSDGGNSAYVVTPPPIMEGRGVVKRFGTRSHVVPMHSDWFSPATVHRLERQVVPHFFSGKSPDHTPEKYMECRNYVVAKYMENPEKRFTVSDFVGVIPGLETEDLSRIVRFLDHWGIINYCAAVSSHEPWNSSSYLKEESNGEIHVPSAALKSIDSLIKFEKPKCKLKAADVYSSLSSHDNNVSDLDNRIREHLSENRCNSCSRSLPTVCYQSHKEVDIMLCSDCFHDGRYVIGHSSLDFMRMDSTKDYGDPDGESWTDQETLLLLEAVEMYNENWNEIAEHVGTKSKAQCILHFLRLPVEDGLLENIEVPGNPISSSMSNGAVHGQIIANGDSAGSCQQDADFESRFPFANSGNPVMSLVAFLASSVGPRVAAACAHASLAALSEESSLSASENLLHKEGPGQSNRMNSEIIHGRDGGPQGEIANSAHHKAVASRGQNEGATPLSAEKVKAAAKVGLAAAATKAKLFADHEEREIQRLSANIINHQLKRLELKLKQFAEVETFLMKXCEQVERTRQRLLAERSRFMAAQMGASGITSMTLPGAPGPSMVNNSGNNRQQLMSAAPSQPSISGYNNNHQPQQVHPHMPFRPRQQMFAMGPRMPLGAI

SEQ ID NO: 37 - protein, 807 aa, Cannabis sativa
MPASEDRRGKWKRKKRGGLSAARKPKQEEEDMEEEDEENNNNNNEEMDDVENADELQQNGGATPDPGLGIGEVVEDSGSRISDFPAVVKRVVIRPHASVMAVVAAERAGLIGETRGQGSLPALENISFGQLQALSTVPADSLDLERSDGSSSAYVISPPPIMDGEGVVKRFGDLVHVLPMHSDWFAPNTVDRLERQVVPQFFSGKSPNHTPESYMEFRNAIVSKYVENPEKTLTISDCQGLVDGVDIEDFARVFRFLDHWGIINYCATAQSHPGPLRDVSDVREDTNGEVNVPSAALTSIDSLIKFDKPNCRHKGGEVYSSLPSLDGDSPDLDIRIREHLCDSHCNHCSRPLPTVYFQSQKKGDILLCCDCFHHGRFVVGHSCLDFVRVDPMKFYGDQDGDNWTDQETLLLLEAVELYNENWVQIADHVGSKSKAQCILHFLRLPVEDGLLDNVEVSGVTNTENPTNGYDHKGTDSNGDLPGYSEQGSDTEIKLPFVKSPNPVMALVAFLASAVGPRVAASCAHESLSVLSEDDRMKSEGMQGKEASLLDGENQQQDGAHKTSSQNGAEAQTPLPQDKVMAAFRAGLSAAATKAKLFADHEEREIQRLSANIVNHQLKRMELKLKQFAEIETLLMKECEQVEKTRQRFSAERARMLSARFGSPGGISPQTNNLQGMSLSTGGNNINSLMHQQHQQQQASATSQPSIIPGFSNNPQVQAQMHFMARQQQQQQQQQQQQQQAFSFGPRLPLNAIQTNAGSTASPNVMFGNNQLNNPAAAGAASINQPSFSHPMVRSSTGSGSGSGLGLN

SEQ ID NO: 38 - DNA, 101 bp, Cannabis sativa
  source 1..101: mol_type = other DNA
catggaatgtaggaactatgttgttgccaaatacatggagaatccagaaaaacggttcactgtgtctgatttcgtgggagtgatacctggtcttgaaactg

SEQ ID NO: 39 - DNA, 101 bp, Cannabis sativa
  source 1..101: mol_type = other DNA
ttgcttccatgatggaaggtatgtgattggtcactcaagcttagattttatgaggatggattcaacaaaagattatggcgatccagatggggaaagttgga

SEQ ID NO: 40 - DNA, 500 bp, Cannabis sativa
  source 1..500: mol_type = other DNA
gagtggacagcaggcacgtatgcaagatgtttttaagattcgaaatatgcaagaaggaactaaatggcgaaaagtgcagattataaatatggaatgatgaataactcctgtctactatcttgtgtacttttcttctgtaatcttatgttttgtgttgttatattatgttatgtaactctttatgtaaactatttattaaacttaataaatactggtcatgtgcaccagtttcatatcctttggtttcctttgctgaactgttcccattcagccaaatgaatgtgtcactgtattaatagggttgattatgcaccccaaatcaaactttaatccctttcattatctcaagcaagatttgttagataaaatggttcaagggtatatatctgatttctgatattagttatcataacaacaggtgattgacttaacatttaaccttttgtgtttgatatacagaaagagactggcctttgatttgattatgctgattggtgctt

SEQ ID NO: 41 - DNA, 495 bp, Cannabis sativa
  source 1..495: mol_type = other DNA
gagtggacagcaggcacgtatgcaagatgtttttaagattcgaaatatgcaagaaggaactaaatggcgaaaagtgcagattataaatatggaatgatgaataactcctgtctactatcttgtgtacttttcttctgtaatcttatgttttgtgttgttatattatgttatgtaactctttatgtaaactatttattaaacttaataaatactggtcatgtgcaccagtttcatatcctttggtttcctttgctgaactgttcccattcagccaaatgaatgtgtcactgtattaatagggttgattatgcaccccaaatcaaactttaatccctttcattatctcaagcaagatttgttagataaaatggttcaagggtatatatctgatttctgatattagttatcataacaacaggtgattgacttaacatttaaccttttgtgtttgatatacagaaagagactggcctttgatttgattatgctgattgg

SEQ ID NO: 42 - DNA, 488 bp, Cannabis sativa
  source 1..488: mol_type = other DNA
gagtggacagcaggcacgtatgcaagatgtttttaagattcgaaatatgcaagaaggaactaaatggcgaaaagtgcagattataaatatggaatgatgaataactcctgtgtactatcttgtgtacttttcttctgtaatcttatgttttgtgttgttatattatgttatgtaactctttatgtacactatttattaaacttaataaatactggtcatgtgcaccagtttcatatcctttggtttcctttgctgaactgttcccattcagccaaatgaatgtgtcactgtattaatagggtagattatgcaccccaaatcaaactttaatcccttttattatctcaagcaagatttgttagataaaatggttcaagggtatatatctgatattagttatcataacaacaggtgattgacttaacatttaaccttttgtgtttgatatacagaaagagactggcctttgatttgattatgctgattgg

SEQ ID NO: 43 - DNA, 489 bp, Cannabis sativa
  source 1..489: mol_type = other DNA
gagtggacagcaggcacgtatgcaagatgtttttaagatacgaactatgcaagaaggaactaaatggcgaaaagtgcagattataaatatggaatgatgaataactcctgtgtactatcttgtgtacttttcttctgtaatcttatgttttgtgttgttatattatgttatgtaactctttgtgtacactatttattaaacttaataaatactggtcatgtgcaccagtttcatatcctttggtttcctttgctgaactgttcccattcagccaaatgaatgtgtcaatgtattaatagggttgattatgcaccccaaatcaaacttaaatcccttttattatctcaagcaagatttgttagatcaaatggttcaagggtatctgatattagttatcataacaccaggtgattgacttaacatttaaccttttgtgtttaatatacagaaagagactggcctttgatttgattatgctgattggtgctt

SEQ ID NO: 44 - DNA, 493 bp, Cannabis sativa
  source 1..493: mol_type = other DNA
gagtggacagcaggcacgtatgcaagatgtttttaagattcgaaatatgcaagaaggaactaaatggcgaaaagtgcagattataaatatggaatgatgaataactcctgtgtactatcttgtgtacttttcttctgtaatcttatgttttgtgttgttatattatgttatgtaactctttatgtacactatttattaaacttaataaatactggtcatgtgcaccagtttcatatcctttggtttcctttgctgaactgttcccattcagccaaatgaatgtgtcactgtattaatagggtagattatgcaccccaaatcaaactttaatcccttttattatctcaagcaagatttgttagataaaatggttcaagggtatatatctgatattagttatcataacaacaggtgattgacttaacatttaaccttttgtgtttgatatacagaaagagactggcctttgatttgattatgctgattggtgctt

SEQ ID NO: 45 - DNA, 101 bp, Cannabis sativa
  source 1..101: mol_type = other DNA
ctgtattaatagggtagattatgcaccccaaatcaaactttaatcccttttattatctcaagcaagatttgttagataaaatggttcaagggtatatatct

SEQ ID NO: 46 - DNA, 99 bp, Cannabis sativa
  source 1..99: mol_type = other DNA
ctcaagcaagatttgttagataaaatggttcaagggtatatatctgatatagttatcataacaacaggtgattgacttaacatttaaccttttgtgttt

SEQ ID NO: 47 - DNA, 45 bp, Cannabis sativa
  source 1..45: mol_type = other DNA
gtagttatggaccggatccgcagaattagtatatctcacagttaa

SEQ ID NO: 48 - DNA, 54 bp, Cannabis sativa
  source 1..54: mol_type = other DNA
gtagttatggaccggatccgcagaattaccgaaactagtatatctcgcagttaa

SEQ ID NO: 49 - DNA, 54 bp, Cannabis sativa
  source 1..54: mol_type = other DNA
gtagttatggaccggatctgcagaattaccgaaagtagtatatctcacagttaa

SEQ ID NO: 50 - DNA, 1518 bp, Cannabis sativa
  source 1..1518: mol_type = other DNA
atgcgcctcattgtgccactccaaggtgtggtgcaaggtagaggcggccttatttggggctctcttatcccctgcgccctcttctacttcctccagttctacctcaaacgaaaccgtcctccaagtacctcctcgaatcctccgtcgccatctcagtcttcctccaatcttctcgaactccaaaggtcgtcttccaggtcaaacttgtccagtcgtggatccataggccgagtcacactatccacccggggaagtttgattgcaaaaccaaatgactccccttactatattgggctggataaggttagggaggatccttatgacagattgggtaacccaaatggggtaattgagctcggtttggccgagaacaagttgagttttgatcttattgagaaatgggtttctgggaattggactaattcgataattggaaccaatggtggcgatttgaatattagcagcattgcagcataccaaccgttcgatggattgactgaactgaaagtggctattgctgatttcatgactcgggtcgtggaaagaactatcacatttgacccgtctcaaatggtgttaactgctggagcaacccctgctactgaaattttaagcttttgcttagcagacaatggaaacgcatttcttgttcccacaccttattatcccggcttcgatagagacattagatggcgagctggggtggagctaattcctgttcattgccgtagcgctgataactttgctttgaacattactgtgcttgatcaagcatttaatcaggcaagaaagcgaggagtaaaagttcgtggaattctaatttcaaatcctgttaatcctgttggcaatgtactgtctcgggaaacactttgtggtattttagactttgcccaggagaagaacatccatgttatatctgatgaaatatttgctgggtccatgtatggaaatgaggagtttgtgagcatggcagaaattattgagacagagaattttgaaaagagcagggttcacataatatacggactgtcgaaagacctatctattccgggctttagagttggtgttatttattcttacaacgagaatgttttggccgctgcaaaaaagttaacaagattttcttcaatctctgctccggcccagcgtcttctagtatcgatgcttttagatacgagattccttgaggattatatgaagataaacaaagaaaggatacgtaatatgcataacttatttgtagcaggtttggggcaattaggtatcaagtgcgcaaagagtagtgctggtttctactgttgggcaaatatgagtgggtttattccatcatacagtgagaaaggggaacttgagttatgggaaaagctgttgaatgtttctaagatcaatgtaactcctggttcagcttgccactgcatagagccgggctggtttcgttgttgttttactactatagctgaagatgatatccctgtagttatggaccggatccgcagaattagtatatctcacagttaa

SEQ ID NO: 51 - protein, 14 aa, Cannabis sativa
VVMDRIRRISISHS

SEQ ID NO: 52 - protein, 17 aa, Cannabis sativa
VVMDRIRRITETSISRS

SEQ ID NO: 53 - protein, 17 aa, Cannabis sativa
VVMDRICRITESSISHS

SEQ ID NO: 54 - protein, 505 aa, Cannabis sativa
MRLIVPLQGVVQGRGGLIWGSLIPCALFYFLQFYLKRNRPPSTSSNPPSPSQSSSNLLELQRSSSRSNLSSRGSIGRVTLSTRGSLIAKPNDSPYYIGLDKVREDPYDRLGNPNGVIELGLAENKLSFDLIEKWVSGNWTNSIIGTNGGDLNISSIAAYQPFDGLTELKVAIADFMTRVVERTITFDPSQMVLTAGATPATEILSFCLADNGNAFLVPTPYYPGFDRDIRWRAGVELIPVHCRSADNFALNITVLDQAFNQARKRGVKVRGILISNPVNPVGNVLSRETLCGILDFAQEKNIHVISDEIFAGSMYGNEEFVSMAEIIETENFEKSRVHIIYGLSKDLSIPGFRVGVIYSYNENVLAAAKKLTRFSSISAPAQRLLVSMLLDTRFLEDYMKINKERIRNMHNLFVAGLGQLGIKCAKSSAGFYCWANMSGFIPSYSEKGELELWEKLLNVSKINVTPGSACHCIEPGWFRCCFTTIAEDDIPVVMDRIRRISISHS

SEQ ID NO: 55 - DNA, 199 bp, Cannabis sativa
  source 1..199: mol_type = other DNA
attatttgttcatttattgttttattttttctttcttcttctcaatcaccttgttttacaattttgaactcttttgggctttcagccttagaagtaagggatttagatataaagttttctggtgaagtttcatataatatcattatcagctaaaatctcttttacaatgcactttttcaattaatttaggataagaaaa

SEQ ID NO: 56 - DNA, 195 bp, Cannabis sativa
  source 1..195: mol_type = other DNA
attatttgttcatttattgttttattttttcttcttctcaatcaccttgttttacaattttgaactcttttgggctttcagccttagaagtaagggatttagatataaagttttctggtgaagtttcatataatatcattatcagctaaaatctcttttacaatgcactttttcaattaatttaggataagaaaa

SEQ ID NO: 57 - DNA, 195 bp, Cannabis sativa
  source 1..195: mol_type = other DNA
attatttgttcatttattgttttattttttcttcttctcaatcaccttgttttacaattttgaactcttttgggctttcagccttagaagtaagggatttagatataaagttttttggtgaagtttcatataatatcattatcagctaaaatctcttttacaatgcactttttcaattaatttaggataagaaaa

SEQ ID NO: 58 - DNA, 199 bp, Cannabis sativa; identical to SEQ ID NO: 55
  source 1..199: mol_type = other DNA

SEQ ID NO: 59 - DNA, 100 bp, Cannabis sativa
  source 1..100: mol_type = other DNA
actatagctgaagatgatatccctgtagttatggaccggatccgcagaattagtatatctcacagttaaattatttgttcatttattgttttattttttc

SEQ ID NO: 60 - DNA, 104 bp, Cannabis sativa
  source 1..104: mol_type = other DNA
gcagaattagtatatctcacagttaaattatttgttcatttattgttttattttttctttcttcttctcaatcaccttgttttacaattttgaactcttttggg

SEQ ID NO: 61 - DNA, 101 bp, Cannabis sativa
  source 1..101: mol_type = other DNA
ttagtatatctcacagttaaattatttgttcatttattgttttattttttctttcttcttctcaatcaccttgttttacaattttgaactcttttgggctt

SEQ ID NO: 62 - DNA, 101 bp, Cannabis sativa
  source 1..101: mol_type = genomic DNA
ttcctcgtttttcaccaaccgagtactgaattcttcgtcttctagttcctgtacacttcacactcacggggcaactgtccaaatccgaaagctagctcagg

SEQ ID NO: 63 - DNA, 101 bp, Cannabis sativa
  source 1..101: mol_type = genomic DNA
catcacgggccaactctcgacctcgtcatcagtcctattagaatacgtttgcccactttcttccttttgggatcatgttcggccttgatgggttgttggtc

SEQ ID NO: 64 - DNA, 101 bp, Cannabis sativa
  source 1..101: mol_type = genomic DNA
aaaagcatctgcaaaggtgatgttaaagaataggctttgtgttagttagaattagttactttgctctcacggttgagttagttaggttgtttagattttgt

SEQ ID NO: 65 - DNA, 101 bp, Cannabis sativa
  source 1..101: mol_type = genomic DNA
aacccgtacagttccctactttaaaagccaaacggttgccgccctcatatctattaattaaggcttctcaattagccggtgattgacgggtattctactct

SEQ ID NO: 66 - DNA, 101 bp, Cannabis sativa
  source 1..101: mol_type = genomic DNA
gaatataatgtttacctaatttggactatttaattatattctctatattaacagggcttgaagaggactacactctagagaagaatgtaaaagcttttgag

SEQ ID NO: 67 - DNA, 101 bp, Cannabis sativa
  source 1..101: mol_type = genomic DNA
aagacttcagttagattcaattgttcgtcgtcgggtggttgactgcatttctaaactccgagctagattttcatagaatgttctttttttttccttgacat

SEQ ID NO: 68 - DNA, 101 bp, Cannabis sativa
  source 1..101: mol_type = genomic DNA
tatgaaaatgaagcataatttcttgtctctgacacatgcaaggacgattccttgacttaaaaataggcaatggaagaccctaactcagacaagtggagcaa

SEQ ID NO: 69 - DNA, 101 bp, Cannabis sativa
  source 1..101: mol_type = genomic DNA
accaatcatcaagaagaaaaattatgattacatttcattttaggactcctgattgaattgaattgaatacccttttcctcctcagctagagcgaaggccat

SEQ ID NO: 70 - DNA, 101 bp, Cannabis sativa
  source 1..101: mol_type = genomic DNA
gaatttgaaaaatcaagtactatctagtatagaaaatcgttaatccatttaatggccccttcccataaacttagccaggcaatgaagtgtacatggtaaaa

SEQ ID NO: 71 - DNA, 101 bp, Cannabis sativa
  source 1..101: mol_type = genomic DNA
gttcatcttggaactcgttcgtggggatccaatccaattcatgccatattggaaggatatagagtgcatatacatggtcatcttcttggaccgacagaaac

SEQ ID NO: 72 - DNA, 101 bp, Cannabis sativa
  source 1..101: mol_type = genomic DNA
aaacgacgtggaaataaaaaatttgtaccagacagtgaagatgttgtgaagaaagcagaacccttgagtgacaaaccttctacgacacacaatactacccg

SEQ ID NO: 73 - DNA, 101 bp, Cannabis sativa
  source 1..101: mol_type = genomic DNA
ttcatcgaaaatccagcttagacttcccaaccgcccaaagtaacacctgtcatattacaaaatcaattaattattaaataattgacagccaaactaaccaa

SEQ ID NO: 74 - DNA, 101 bp, Cannabis sativa
  source 1..101: mol_type = genomic DNA
tgcggcatgacgaatgagaaccaaagaaaaagggaaagaacgtgatatagctagttgattggaggaataggtttgtgacaaagatataggagattgcattg

SEQ ID NO: 75 - DNA, 101 bp, Cannabis sativa
  source 1..101: mol_type = genomic DNA
tggtgtgagataccgatcttgggcaccagacaggtacatgcatatgtggtcattttaccatgtcaaaaacagtattgaagtgtttggtttcatttgtatgc

SEQ ID NO: 76 - DNA, 101 bp, Cannabis sativa
  source 1..101: mol_type = genomic DNA
tctgggttaatcttatagcgttgatgatttctgactcagagcctttggtattaaaagttcaggatgtagcgtgatcgagtttggaaaaaaatagttgcagc

SEQ ID NO: 77 - DNA, 101 bp, Cannabis sativa
  source 1..101: mol_type = genomic DNA
cactctaatagcataccaaattatacactaaaatgttgtacactaaatttgacatcattaatagtgttgaatactgaatctataccaatttaattttagtg

SEQ ID NO: 78 - DNA, 101 bp, Cannabis sativa
  source 1..101: mol_type = genomic DNA
aagtactagctgacattctctaactaattaaaataatcatatattcattcccaacttcctttcttctttaactttccattgccaatggactaataattttt

SEQ ID NO: 79 - DNA, 101 bp, Cannabis sativa
  source 1..101: mol_type = genomic DNA
aaatccgttagaccttaccgtcattacaaaagataatatcggacatagaggatagaggtatggagccgaacctttgacaagaaaacgtctatttggccaca

SEQ ID NO: 80 - DNA, 101 bp, Cannabis sativa
  source 1..101: mol_type = genomic DNA
gagtctacacttctagccacacatgcatatatctttttctttgtcatgctgttatgttatgtaaagaaaaattgaacttgtctagaggaaaaagtggctct

(19) United States
(12) Patent Application Publication
(10) Pub. No.: US 2026/0250785 A1
(43) Pub. Date: Aug. 27, 2026

(54) HERMAPHRODITISM MARKERS

(71) Applicant: Phylos Bioscience, Inc., Portland, OR (US)

(72) Inventors: Erica Bakker, Portland, OR (US); Alisha Holloway, Portland, OR (US)

(73) Assignee: Phylos Bioscience, Inc., Portland, OR (US)

(21) Appl. No.: 18/728,344

(22) PCT Filed: Jan. 11, 2023
(86) PCT No.: PCT/US2023/060493

§ 371 Date: Jul. 11, 2024

Related U.S. Application Data
(60) Provisional application No. 63/298,591, filed on Jan. 11, 2022.

(51) Int. Cl. C12Q 1/6895; C12N 15/82

(52) U.S. Cl. CPC C12Q 1/6895; C12N 15/8287; C12Q 2600/13; C12Q 2600/156

(57) ABSTRACT

Provided herein is the identification of markers and genes associated with hermaphroditism susceptibility and resistance in Cannabis. The markers are useful for breeding Cannabis plants having resistance to hermaphroditism, and methods for selecting plants by obtaining nucleic acids and detecting one or more markers that indicate resistance to hermaphroditism to establish plant lines having such resistance are provided.

### CROSS-REFERENCE TO RELATED APPLICATION

This application claims priority to U.S. Provisional Application No. 63/298,591 filed Jan. 11, 2022, herein incorporated by reference in its entirety.

### FIELD

The present disclosure relates to markers and genes associated with hermaphroditism susceptibility and resistance in cannabis, and methods for selecting cannabis plants resistant to hermaphroditism.

### BACKGROUND

This application is directed to the field of hermaphroditism resistance in cannabis. In particular, identifying genes and markers involved in susceptibility and resistance to the production of male and/or hermaphroditic flowers on a genetically female plant, herein referred to as “hermaphroditism.”

Cannabis usually occurs as either female or male plants (dioccious) as determined by their sex chromosomes, XX for female and XY for male; however, some hemp varieties are monecious (Moliterni et al., Euphytica 140:95-106 (2004)). In monecious hemp quantitative trait loci (QTL) for the ratio of female to male flowers per plant have been identified on the X chromosome (Faux, A-M., et al., Euphytica 209.2::357-376 (2016)). Cannabis plants that are genetically female usually bear female flowers, although genetic makeup, environmental stressors, developmental cues, and application of growth hormones or certain chemicals can result in the appearance of male and/or hermaphroditic flowers on genetically female plants (Grant et al., Dev Genet 15:214-230 (1994). Hermaphroditic flowers are pistillate flowers which are accompanied by anthers; plants grown from seed formed on hermaphroditic plants are genetically female (Punja and Holmes. Frontiers in Plant Science 11:718 (2020)).

Both commercial production of cannabis flower and breeding cannabis plants require strict control of pollen. In commercial production of cannabis flower, female flowers are desired as they are the only flowers that produce appreciable quantities of valuable cannabinoids. Female plants bearing hermaphroditic and/or male flowers lead to pollination of female flowers and subsequent seed production, which severely reduces the value of the crop. In breeding, the unwanted production of male and/or hermaphroditic flowers on a plant designated as a pollen receiver leads to undesired genetic combinations. Thus, genetically female plants that have a predisposition to produce hermaphroditic and/or male flowers are as undesirable as genetically male plants because both produce pollen.

The methods described herein solve the laborious and time-consuming issues of traditional breeding methods by providing cannabis breeders with a specific and efficient method for creating cannabis plants having resistance to producing hermaphroditic and/or male flowers on genetically female plants.

### SUMMARY

The present teachings relate to methods of selecting plants with resistance to hermaphroditism. Methods for selecting one or more plants having resistance to hermaphroditism are provided. In one implementation, the method comprises i) obtaining nucleic acids from a sample plant or its germplasm; (ii) detecting one or more markers that indicate resistance to hermaphroditism, and (iii) indicating resistance to hermaphroditism. The method optionally further comprises selecting the one or more plants indicating resistance to hermaphroditism. In some examples, the one or more markers comprise a polymorphism relative to a reference genome at nucleotide position: (a) 5,705,332 on chromosome X: (b) 5,732,323 on chromosome X; (c) 5,747,057 on chromosome X; (d) 5,877,981 on chromosome X; (e) 5,920,712 on chromosome X, (f) 6,053,325 on chromosome X, (g) 6,181,263 on chromosome X, (h) 6,186,518 on chromosome X, (1) 6,192,534 on chromosome X, (j) 6,261,819 on chromosome X, (k) 6,285,113 on chromosome X, (1) 6,695,193 on chromosome X, (m) 6,961,002 on chromosome X, (n) 17,971,672 on chromosome X, (0) 14,810,444 on chromosome 3, (p) 57,051,092 on chromosome 3, (q) 74,435,555 on chromosome 3, (r) 5,233,698 on chromosome 4, or(s) 12,961,444 on chromosome 4, wherein the reference genome is the Abacus Cannabis reference genome Csat_AbacusV2; NCBI assembly accession GCA 025232715.1 (CsaAba2) In a non-limiting example, the one or more markers comprise a polymorphism at one or more of nucleotide position 5,732,323 on chromosome X, nucleotide position 17,971,672 on chromosome X, and nucleotide position 14,810,444 on chromosome 3. In another non-limiting example, the one or more markers comprise a polymorphism at nucleotide position 5,732,323 on chromosome X, nucleotide position 17,971,672 on chromosome X, and nucleotide position 14,810,444 on chromosome 3. In a further non-limiting example, the one or more markers consist of, or essentially consist of, a polymorphism at nucleotide position 5,732,323 on chromosome X, nucleotide position 17,971,672 on chromosome X, and nucleotide position 14,810,444 on chromosome 3. In some examples, the one or more nucleotide positions comprise: (a) on chromosome X: (1) an A/A or G/A genotype at position 5,705,332; (2) an A/A or G/A genotype at position 5,732,323; (3) a G/G or A/G genotype at position 5,747,057; (4) a T/T or C/T genotype at position 5,877,981; (5) a C/C or A/C genotype at position 5,920,712; (6) a T/T or C/T genotype at position 6,053,325; (7) a T/T or C/T genotype at position 6,181,263; (8) an A/A or G/A genotype at position 6,186,518; (9) a C/C or A/C genotype at position 6,192,534; (10) an A/A or G/A genotype at position 6,261,819; (11) an A/A or G/A genotype at position 6,285,113; (12) an A/A or C/A genotype at position 6,695,193; (13) a T/T or C/T genotype at position 6,961,002; or (14) a T/T or C/T genotype at position 17,971,672; (b) on chromosome 3: (1) a T/T or C/T genotype at position 14,810,444; (2) a G/G or G/A genotype at position 57,051,092; or (3) a C/C genotype at position 74,435,555; (c) on chromosome 4: (1) a G/G or G/A genotype at position 5,233,698; or (2) a G/G genotype at position 12,961,444; wherein the reference genome is the Abacus Cannabis reference genome Csat_AbacusV2; NCBI assembly accession GCA_025232715.1 (CsaAba2). In a non-limiting example, the one or more nucleotide positions comprise an A/A or G/A genotype at position 5,732,323 on chromosome X, a T/T or C/T genotype at position 17,971,672, or a T/T or C/T genotype at position 14,810,444. In another non-limiting example, the one or more nucleotide positions comprise an A/A or G/A genotype at position 5,732,323 on chromosome X, a T/T or C/T genotype at position 17,971,672 on chromosome X, and a T/T or C/T genotype at position 14,810,444 on chromosome 3. In a further non-limiting example, the one or more nucleotide positions consist of, or essentially consist of, an A/A or G/A genotype al position 5,732,323 on chromosome X, a T/T or C/T genotype at position 17,971,672 on chromosome X, and a T/T or C/T genotype at position 14,810,444 on chromosome 3.

In some examples, the one or more markers comprises a polymorphism at position 26 of any one or more of SEQ ID NO:1: SEQ ID NO:2; SEQ ID NO:3; SEQ ID NO:4; SEQ ID NO:5; SEQ ID NO:6; SEQ ID NO: 7; SEQ ID NO:8; SEQ ID NO:9; SEQ ID NO:10; SEQ ID NO:11; SEQ ID NO:12; SEQ ID NO:13; SEQ ID NO:14; SEQ ID NO:15; SEQ ID NO:16; SEQ ID NO:17; SEQ ID NO:18; or SEQ ID NO:19. In some examples, the nucleotide position comprises: (1) an A/A or G/A genotype at position 26 of SEQ ID NO: 1; (2) an A/A or G/A genotype at position 26 of SEQ ID NO:2; (3) a G/G or A/G genotype at position 26 of SEQ ID NO:3; (4) a T/T or C/T genotype at position 26 of SEQ ID NO:4; (5) a C/C or A/C genotype at position 26 of SEQ ID NO:5; (6) a T/T or C/T genotype at position 26 of SEQ ID NO:6; (7) a T/T or C/T genotype at position 26 of SEQ ID NO:7; (8) an A/A or G/A genotype at position 26 of SEQ ID NO:8; (9) a C/C or A/C genotype at position 26 of SEQ ID NO:9; (10) an A/A or G/A genotype at position 26 of SEQ ID NO: 10; (11) an A/A or G/A genotype at position 26 of SEQ ID NO:11; (12) an A/A or C/A genotype at position 26 of SEQ ID NO: 12; (13) a T/T or C/T genotype at position 26 of SEQ ID NO:13; (14) a T/T or C/T genotype at position 26 of SEQ ID NO: 14; (15) a T/T or C/T genotype at position 26 of SEQ ID NO: 15; (16) a G/G or G/A genotype at position 26 of SEQ ID NO:16; (17) a C/C genotype at position 26 of SEQ ID NO: 17; (18) a G/G or G/A genotype at position 26 of SEQ ID NO:18; or (19) a G/G genotype at position 26 of SEQ ID NO:19; wherein the reference genome is the Abacus Cannabis reference genome Csat_AbacusV2; NCBI assembly accession GCA_025232715.1 (CsaAba2). In a non-limiting example, the nucleotide position comprises an A/A or G/A genotype at position 26 of SEQ ID NO:2; a T/T or C/T genotype at position 26 of SEQ ID NO:14; or a T/T or C/T genotype at position 26 of SEQ ID NO:15. In another non-limiting example, the nucleotide position comprises at least two nucleotide positions selected from: an A/A or G/A genotype at position 26 of SEQ ID NO:2; a T/T or C/T genotype at position 26 of SEQ ID NO: 14; and a T/T or C/T genotype at position 26 of SEQ ID NO:15. In a further non-limiting example, the nucleotide position comprises at least three nucleotide positions: an A/A or G/A genotype at position 26 of SEQ ID NO:2; a T/T or C/T genotype at position 26 of SEQ ID NO:14; and a T/T or C/T genotype at position 26 of SEQ ID NO:15.

In some examples, the one or more markers comprise a polymorphism at position 51 of any one or more of SEQ ID NO:62; SEQ ID NO:63; SEQ ID NO:64; SEQ ID NO:65; SEQ ID NO:66; SEQ ID NO:67; SEQ ID NO:68; SEQ ID NO:69; SEQ ID NO:70; SEQ ID NO:71; SEQ ID NO:72; SEQ ID NO:73; SEQ ID NO: 74; SEQ ID NO:75; SEQ ID NO:76; SEQ ID NO:77; SEQ ID NO:78; SEQ ID NO:79; or SEQ ID NO: 80. In some examples, the nucleotide position comprises: (1) an A/A or G/A genotype at position 51 of SEQ ID NO:62; (2) an A/A or G/A genotype at position 51 of SEQ ID NO:63; (3) a G/G or A/G genotype at position 51 of SEQ ID NO:64; (4) a T/T or C/T genotype at position 51 of SEQ ID NO:65; (5) a C/C or A/C genotype at position 51 of SEQ ID NO:66; (6) a T/T or C/T genotype at position 51 of SEQ ID NO:67; (7) a T/T or C/T genotype at position 51 of SEQ ID NO:68; (8) an A/A or G/A genotype at position 51 of SEQ ID NO: 69; (9) a C/C or A/C genotype at position 51 of SEQ ID NO:70; (10) an A/A or G/A genotype at position 51 of SEQ ID NO:71; (11) an A/A or G/A genotype at position 51 of SEQ ID NO:72; (12) an A/A or C/A genotype at position 51 of SEQ ID NO:73; (13) a T/T or C/T genotype at position 51 of SEQ ID NO: 74; (14) a T/T or C/T genotype at position 51 of SEQ ID NO:75; (15) a T/T or C/T genotype at position 51 of SEQ ID NO:76; (16) a G/G or G/A genotype at position 51 of SEQ ID NO:77; (17) a C/C genotype at position 51 of SEQ ID NO:78; (18) a G/G or G/A genotype at position 51 of SEQ ID NO:79; or (19) a G/G genotype at position 51 of SEQ ID NO:80; wherein the reference genome is the Abacus Cannabis reference genome Csat_AbacusV2; NCBI assembly accession GCA_025232715.1 (CsaAba2). In a non-limiting example, the nucleotide position comprises an A/A or G/A genotype at position 51 of SEQ ID NO:63; a T/T or C/T genotype at position 51 of SEQ ID NO:75; or a T/T or C/T genotype at position 51 of SEQ ID NO: 76. In another non-limiting example, the nucleotide position comprises at least two nucleotide positions selected from: an A/A or G/A genotype at position 51 of SEQ ID NO:63; a T/T or C/T genotype at position 51 of SEQ ID NO:75; and a T/T or C/T genotype at position 51 of SEQ ID NO:76. In a further non-limiting example, the nucleotide position comprises at least three nucleotide positions: an A/A or G/A genotype at position 51 of SEQ ID NO:63; a T/T or C/T genotype at position 51 of SEQ ID NO:75; and a T/T or C/T genotype at position 51 of SEQ ID NO:76.

In some examples, the one or more markers comprises a polymorphism relative to a reference genome within any one or more haplotypes wherein the haplotypes comprise the region: (a) on chromosome X: (1) between positions 5,696,400 and 5,714,336; (2) between positions 5,725,231 and 5,737,968; (3) between positions 5,737,968 and 5,750,301; (4) between positions 5,860,363 and 5,894,065; (5) between positions 5,894,065 and 5,929,330; (6) between positions 6,032,408 and 6,059,371; (7) between positions 6,133,760 and 6,189,246; (8) between positions 6,189,246 and 6,197,726; (9) between positions 6,258,772 and 6,290,824; (10) between positions 6,688,670 and 6,715,018; (11) between positions 6,932,849 and 6,970,023; (12) between positions 17,900,648 and 17,980,443; (b) on chromosome 3: (1) between positions 14,797,288 and 14,824,861; (2) between positions 56,616,473 and 57,090,960; or (3) between positions 74,422,393 and 74,437,841; (c) on chromosome 4: (1) between positions 5,216,694 and 5,243,017; or (2) between positions 12,923,613 and 13,009,666; wherein the reference genome is the Abacus Cannabis reference genome Csat_AbacusV2; NCBI assembly accession GCA_025232715.1 (CsaAba2). In a non-limiting example, the haplotype includes the region between positions 5,725,231 and 5,737,968 on chromosome X, the region between positions 17,900,648 and 17,980,443 on chromosome X, or the region between positions 14,797,288 and 14,824,861 on chromosome 3. In another non-limiting example, the haplotype includes at least two of the regions between positions 5,725,231 and 5,737,968 on chromosome X, positions 17,900,648 and 17,980,443 on chromosome X, and positions 14,797,288 and 14,824,861 on chromosome 3. In a further non-limiting example, the haplotype includes the region between positions 5,725,231 and 5,737,968 on chromosome X, the region between positions 17,900,648 and 17,980,443 on chromosome X, and the region between positions 14,797,288 and 14,824,861 on chromosome 3.

In some implementations, the methods include marker assisted selection. In some implementations, detecting comprises using an oligonucleotide probe or primer. In some implementations, detecting comprises detection by sequencing. In some implementations, the methods further comprise crossing one or more plants comprising resistance to hermaphroditism to produce one or more F1 or additional progeny plants, wherein at least one of the F1 or additional progeny plants comprise resistance to hermaphroditism. In some examples, crossing comprises selfing, sibling crossing, outcrossing, or backcrossing. In some implementations, the F1 or additional progeny plants comprising resistance to hermaphroditism comprise an F2-F7 progeny plant. In some examples, the selfing, sibling crossing, outcrossing, or backcrossing comprises marker-assisted selection. In another example, the selfing, sibling crossing, outcrossing, or backcrossing comprises marker-assisted selection for at least two generations. In some examples, the plant comprises a Cannabis plant.

Also included are methods of identifying or selecting a plant having resistance to hermaphroditism. In some implementations, the method includes (i) obtaining a nucleic acid sample from the plant or its germplasm; (ii) detecting a polymorphism at positions 5,732,323 on chromosome X, 17,971,672 on chromosome X, or 4,810,444 on chromosome 3, (wherein the reference genome is the Abacus Cannabis reference genome), thereby identifying the plant having resistance to hermaphroditism. In some examples, a polymorphism is detected at positions 5,732,323 on chromosome X, 17,971,672 on chromosome X, and 4,810,444 on chromosome 3. In some examples, the plant identified as having resistance to hermaphroditism is selected, for example, for further propagation or crossing (plant breeding). In some implementations, the method of identifying a plant resistant to hermaphroditism includes (i) obtaining nucleic acids from a plant or its germplasm; (ii) analyzing the sample to detect a nucleic acid polymorphism in: (a) switch/sucrose nonfermenting 3C (SWI3C), (b) catalase 2 (CAT2), or (c) 1-aminocyclopropane-1-carboxylate synthase 12 (ACS12), thereby identifying a plant having resistance to hermaphroditism. In some examples, the polymorphism is detected in SWI3C. In some examples, the polymorphism is detected in CAT2. In further examples, the polymorphism is detected in ACS12. In some examples, analyzing the sample includes analyzing at least two of SWI3C, CAT2, and ACS12. In some examples, analyzing the sample includes analyzing SWI3C, CAT2, and ACS12. In some examples, analyzing the sample consists of, or essentially consists of, analyzing SWI3C, CAT2, and ACS12. In some examples, one or more polymorphisms are detected in at least two of SWI3C, CAT2, and ACS12. In some examples, one or more polymorphisms are detected in SWI3C, CAT2, and ACS12. In some examples, the polymorphism is associated with resistance to hermaphroditism, or increases resistance to hermaphroditism relative to a reference plant, such as a plant without the beneficial polymorphism (e.g., a polymorphism associated with hermaphroditism resistance) or a plant with a detrimental polymorphism (e.g., a polymorphism associated with hermaphroditism).

Also disclosed are methods of producing a genetically engineered plant that is resistant to hermaphroditism, including introducing a genetic modification in SWI3C, CAT2, or ACS12 that increases resistance to hermaphroditism relative to the plant in an unmodified state, or introducing a heterologous gene that increases resistance to hermaphroditism relative to the plant in an unmodified state (e.g., a beneficial allele of SWI3C, CAT2, or ACS12). In some examples, the genetic modification is a nucleic acid substitution, insertion, or deletion. In some examples, the genetic modification is introduced by a gene editing technique (e.g., RNAi, CRISPR/Cas9, ZFN, or TALEN based systems). In some examples, a genetic modification is introduced in two or more of SWI3C, CAT2, and ACS12. In a non-limiting example, a genetic modification is introduced in SWI3C, CAT2, and ACS12. In another non-limiting example, introducing the genetic modification consists of, or essentially consists of, introducing a modification in SWI3C, CAT2, and ACS12. Exemplary genetic modifications include: a substitution at position 728 bp in the coding sequence from the start codon of Abacus SWI3C (position 5,726,701 bp on chromosome X of the Abacus reference genome); a substitution at position 1193 hp in the coding sequence from the start codon of Abacus SWI3C (position 5,727,731 bp on chromosome X of the Abacus reference genome); a substitution at position 338 bp from the start of the 3′UTR of Abacus CAT2 (position 17,972,352 bp on chromosome X of the Abacus reference genome); an insertion starting at 394 bp from the start of the 3′UTR of Abacus CAT2 (position 17,972,408 bp on chromosome X of the Abacus reference genome); a deletion at position 1500 bp from the start codon of Abacus ACS12 (position 14,804,943 bp on chromosome 3 of the Abacus reference genome); an insertion at position 25 bp from the start of the 3′UTR of Abacus ACS12 (position 14,804,986 bp on chromosome 3 of the Abacus reference genome); and a substitution at position 31 bp from the start of the 3′UTR of Abacus ACS12 (position 14,804,992 bp on chromosome 3 of the Abacus reference genome). The Abacus Cannabis reference genome is Csat_AbacusV2, NCBI assembly accession GCA_025232715.1. In a non-limiting example, the substitution at position 728 bp in the coding sequence from the start codon of SWI3C is a G. In a non-limiting example, the substitution at position 1193 bp in the coding sequence from the start codon of SWI3C is a C. In a non-limiting example, the substitution at position 338 bp from the start of the 3′UTR of CAT2 is a C. In a non-limiting example, the insertion starting at 394 bp from the start of the 3′UTR of CAT2 is an insertion of CTGATAT. In a non-limiting example, the deletion at position 1500 bp from the start codon of ACS12 is a deletion of TACCGAAAC or TACCGAAAG. In a non-limiting example, the insertion at position 25 bp from the start of the 3′UTR of ACS12 is TTTT. In a non-limiting example, the substitution at position 31 bp from the start of the 3′UTR of ACS12 is a C.

The disclosure includes a plant made by any of the methods disclosed herein, or any product made therefrom. The plant of any of the methods or compositions disclosed herein can be a cannabis plant.

The disclosure includes a method for selecting one or more plants having resistance to hermaphroditism, including replacing a nucleic acid sequence of a parent plant with a nucleic acid sequence conferring resistance to hermaphroditism.

The foregoing and other features of this disclosure will become more apparent from the following detailed description of several aspects which proceeds with reference to the accompanying figures.

### BRIEF DESCRIPTION OF THE DRAWINGS

FIG. 1 illustrates logistic regression of the prevalence of hermaphroditism in a set of 205 diverse seed lots (n=1317).

### SEQUENCE LISTING

A Sequence Listing XML (submitted under 37 C.F.R. § 1.831 (a) in compliance with §§ 1.832 through 1.834) is submitted herewith as “Sequence.xml,” created on Jan. 11, 2023, 90,112 bytes, which is incorporated by reference herein.

Only one strand of each nucleic acid sequence is shown, but the complementary strand is understood as included by any reference to the displayed strand.

### DETAILED DESCRIPTION

The present teachings relate generally to producing or developing Cannabis varieties having resistance to hermaphroditism by selecting plants having markers indicating such resistance.

Unless otherwise noted, technical terms are used according to conventional usage. As used herein, the singular forms “a”, “an” and “the” are intended to include the plural forms as well, unless the context clearly indicates otherwise. The term “about” refers to an amount within a specific range of a value. For example, “about” a specific gram amount or volume indicates within 10% that amount (unless indicated otherwise). It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various aspects, the following explanations of terms are provided:

### Definitions

The term “Abacus” as used herein refers to the Cannabis reference genome known as the Abacus Cannabis reference genome (Csat_AbacusV2, NCBI assembly accession GCA_025232715.1; also referred to as “CsaAba2”).

The term “alternative nucleotide call” is a nucleotide polymorphism relative to a reference nucleotide for a SNP marker that is significantly associated with the causative SNP(s) that confer(s) a desired phenotype.

The term “backcrossing” or “to backcross” refers to the crossing of an F1 hybrid with one of the original parents. A backcross is used to maintain the identity of one parent (species) and to incorporate a particular trait from a second parent (species), One strategy is to cross the F1 hybrid back to the parent possessing the most desirable traits. Two or more generations of backcrossing may be necessary, but this is practical only if the desired characteristic or trait is present in the F1.

The term “beneficial allele” as used herein refers to an allele conferring a hermaphroditism resistance phenotype.

The term “cannabinoid” refers to the class of compounds found in cannabis. Non-limiting examples include THC and CBD, but can also include any of the other hundred plus distinct cannabinoids isolated from cannabis.

The term “Cannabis” refers to plants of the genus Cannabis, including Cannabis sativa, Cannabis indica, and Cannabis ruderalis.

The term “cell” refers to a prokaryotic or eukaryotic cell, including plant cells, capable of replicating DNA, transcribing RNA, translating polypeptides, and secreting proteins.

The term “coding sequence” refers to a DNA sequence which codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences.

The terms “construct,” “plasmid,” “vector,” and “cassette” refer to an extra chromosomal element often carrying genes that are not part of the central metabolism of the cell, and usually in the form of circular double-stranded DNA fragments. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. The term “recombinant DNA construct” or “recombinant expression construct” is used interchangeably and refers to a discrete polynucleotide into which a nucleic acid sequence or fragment can be moved. Preferably, it is a plasmid vector, or a fragment thereof, comprising the promoters. The choice of plasmid vector is dependent upon the method that will be used to transform host plants. Similarly, genetic elements that must be present on the plasmid vector to successfully transform, select and propagate host cells containing the chimeric gene is dependent on the specific transformation method. Different independent transformation events typically result in different levels and patterns of expression and thus multiple events must be screened to obtain lines displaying the desired expression level and pattern. Such screening may be accomplished by PCR and Southern analysis of DNA, RT-PCR and Northern analysis of mRNA expression, Western analysis of protein expression, or phenotypic analysis.

The term “cross”, “crossing”, “cross pollination” or “cross-breeding” refer to the process by which the pollen of one flower on one plant is applied (artificially or naturally) to the ovule (stigma) of a flower on another plant. Exemplary types of crossing include selfing, sibling crossing, outcrossing, and backcrossing.

The term “cultivar” means a group of similar plants that by structural features and performance (e.g., morphological and physiological characteristics) can be identified from other varieties within the same species. Furthermore, the term “cultivar” variously refers to a variety, strain or race of plant that has been produced by horticultural or agronomic techniques and is not normally found in wild populations. The terms cultivar, variety, strain, plant and race are often used interchangeably by plant breeders, agronomists and farmers.

The term “detect” or “detecting” refers to any of a variety of methods for determining the presence of a nucleic acid.

The term “expression” or “gene expression” relates to the process by which the coded information of a nucleic acid transcriptional unit (including, e.g., genomic DNA) is converted into an operational, non-operational, or structural part of a cell, often including the synthesis of a protein. Gene expression can be influenced by external signals; for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any method known in the art, including, without limitation, Northern blot, RT-PCR, Western blot, or in vitro, in situ, or in vivo protein activity assay(s). Elevated levels refer to higher than average levels of gene expression in comparison to a reference, e.g., the Abacus reference genome.

The term “expression cassette” refers to a discrete nucleic acid fragment into which a nucleic acid sequence or fragment can be moved.

The term “functional” as used herein refers to DNA or amino acid sequences which are of sufficient size and sequence to have the desired function (i.e. the ability to cause expression of a gene resulting in gene activity expected of the gene found in a reference genome, e.g., the Abacus reference genome.)

The term “gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” or “recombinant expression construct”, which are used interchangeably, refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes.

The term “genetic modification” or “genetic alteration” as used herein refers to a change from the wild-type or reference sequence of one or more nucleic acid molecules. Genetic modifications or alterations include without limitation, base pair substitutions, additions and deletions of at least one nucleotide from a nucleic acid molecule of known sequence.

The term “genome” as it applies to plant cells encompasses not only chromosomal DNA found within the nucleus, but organelle DNA found within subcellular components (e.g., mitochondrial, plastid) of the cell.

The term “genotype” refers to the genetic makeup of an individual cell, cell culture, tissue, organism (e.g., a plant), or group of organisms. A “detrimental genotype” is a genotype that is susceptible to hermaphroditism or produces hermaphroditic flowers. Conversely, a “beneficial genotype” refers to a genotype that is resistant to hermaphroditism or does not produce hermaphroditic flowers. A genotype may refer to a particular genetic marker (e.g., a polymorphism), such as a marker associated with resistance or susceptibility to hermaphroditism. Thus, a “detrimental polymorphism” is a polymorphism associated with susceptibility to hermaphroditism or plants that produce hermaphroditic flowers, and a “beneficial polymorphism” refers to a polymorphism that is associated with resistance to hermaphroditism or plants that do not produce hermaphroditic flowers. In some examples, a beneficial genotype or polymorphism increases resistance to hermaphroditism relative to a plant that does not contain the beneficial genotype or polymorphism, or relative to a plant that contains a detrimental genotype or polymorphism.

The term “germplasm” refers to genetic material of or from an individual (e.g., a plant), a group of individuals (e.g., a plant line, variety, or family), or a clone derived from a line, variety, species, or culture. The germplasm can be part of an organism or cell, or can be separate from the organism or cell. In general, germplasm provides genetic material with a specific molecular makeup that provides a physical foundation for some or all of the hereditary qualities of an organism or cell culture. As used herein, germplasm includes cells, seed or tissues from which new plants can be grown, as well as plant parts, such as leafs, stems, pollen, or cells that can be cultured into a whole plant.

The term “haplotype” refers to the genotype of a plant at a plurality of genetic loci, e.g., a combination of alleles or markers. Haplotype can refer to sequence polymorphisms at a particular locus, such as a single marker locus, or sequence polymorphisms at multiple loci along a chromosomal segment in a given genome. As used herein, a haplotype can be a nucleic acid region spanning two markers.

The term “hermaphroditism” refers to female plants bearing hermaphroditic and/or male flowers.

A plant is “homozygous” if the individual has only one type of allele at a given locus (e.g., a diploid individual has a copy of the same allele at a locus for each of two homologous chromosomes). An individual is “heterozygous” if more than one allele type is present at a given locus (e.g., a diploid individual with one copy each of two different alleles). The term “homogeneity” indicates that members of a group have the same genotype at one or more specific loci. In contrast, the term “heterogeneity” is used to indicate that individuals within the group differ in genotype at one or more specific loci.

The term “hybrid” refers to a variety or cultivar that is the result of a cross of plants of two different varieties. A hybrid, as described here, can refer to plants that are genetically different at any particular loci. A hybrid can further include a plant that is a variety that has been bred to have at least one different characteristic from the parent. “F1 hybrid” refers to the first generation hybrid, “F2 hybrid” the second generation hybrid, “F3 hybrid” the third generation, and so on. A hybrid refers to any progeny that is either produced, or developed using research and development to create a new line having at least one distinct characteristic.

The terms “hybridizing specifically to”, “specific hybridization”, and “selectively hybridize to,” as used herein refer to the binding, duplexing, or hybridizing of a nucleic acid molecule preferentially to a particular nucleotide sequence under stringent conditions. The term “stringent conditions” refers to conditions under which a probe will hybridize preferentially to its target subsequence, and to a lesser extent to, or not at all to, other sequences. A “stringent hybridization” and “stringent hybridization wash conditions” in the context of nucleic acid hybridization (e.g., as in array, Southern or Northern hybridizations) are sequence dependent, and are different under different environmental parameters. An extensive guide to the hybridization of nucleic acids is can be found in, e.g., Tijssen (1993) Laboratory Techniques in Biochemistry and Molecular Biology—Hybridization with Nucleic Acid Probes part I, Ch. 2, “Overview of principles of hybridization and the strategy of nucleic acid probe assays,” Elsevier, N.Y. (“Tijssen”). Generally, highly stringent hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on an array or on a filter in a Southern or northern blot is 42° C. using standard hybridization solutions (see, e.g., Sambrook et al. (2014) Molecular Cloning: A Laboratory Manual (Fourth Edition, Cold Spring Harbor Laboratory Press, Plainview, N.Y.), and detailed discussion, below).

As used herein, the term “inbreeding” refers to the production of offspring via the mating between relatives. The plants resulting from the inbreeding process are referred to herein as “inbred plants” or “inbreds.”

The terms “initiate transcription,” “initiate expression,” “drive transcription,” and “drive expression” are used interchangeably herein and all refer to the primary function of a promoter. As detailed throughout this disclosure, a promoter is a non-coding genomic DNA sequence, usually upstream (5′) to the relevant coding sequence, and its primary function is to act as a binding site for RNA polymerase and initiate transcription by the RNA polymerase. Additionally, there is “expression” of RNA, including functional RNA, or the expression of polypeptide for operably linked encoding nucleotide sequences, as the transcribed RNA ultimately is translated into the corresponding polypeptide.

The term “introduced” refers to incorporation of a nucleic acid (e.g., expression construct) or protein into a cell. Introduced includes reference to the incorporation of a nucleic acid into a eukaryotic or prokaryotic cell where the nucleic acid may be incorporated into the genome of the cell, and includes reference to the transient provision of a nucleic acid or protein to the cell. Introduced includes reference to stable or transient transformation methods, as well as sexually crossing. Thus, “introduced” in the context of inserting a nucleic acid fragment (e.g., a recombinant DNA construct/expression construct) into a cell, includes “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA). In some examples, a genetic modification (e.g., a substitution, insertion, or deletion) is introduced through a gene editing technique, such as an RNAi, CRISPR/Cas9, ZFN, or TALEN based technique.

The term “isolated” as used herein means having been removed from its natural environment, or removed from other compounds present when the compound is first formed. The term “isolated” embraces materials isolated from natural sources as well as materials (e.g., nucleic acids and proteins) recovered after preparation by recombinant expression in a host cell, or chemically-synthesized compounds such as nucleic acid molecules, proteins, and peptides.

The term “line” is used broadly to include, but is not limited to, a group of plants vegetatively propagated from a single parent plant, via tissue culture techniques or a group of inbred plants which are genetically very similar due to descent from a common parent(s). A plant is said to “belong” to a particular line if it (a) is a primary transformant (TO) plant regenerated from material of that line; (b) has a pedigree comprised of a TO plant of that line; or (c) is genetically very similar due to common ancestry (e.g., via inbreeding or selfing). In this context, the term “pedigree” denotes the lineage of a plant, e.g. in terms of the sexual crosses affected such that a gene or a combination of genes, in heterozygous (hemizygous) or homozygous condition, imparts a desired trait to the plant.

The term “marker,” “genetic marker,” “molecular marker,” “marker nucleic acid,” and “marker locus” refer to a nucleotide sequence or encoded product thereof (e.g., a protein) used as a point of reference when identifying a linked locus. A marker can be derived from genomic nucleotide sequence or from expressed nucleotide sequences (e.g., from a spliced RNA, a cDNA, etc.), or from an encoded polypeptide, and can be represented by one or more particular variant sequences, or by a consensus sequence. In another sense, a marker is an isolated variant or consensus of such a sequence. The term also refers to nucleic acid sequences complementary to or flanking the marker sequences, such as nucleic acids used as probes or primer pairs capable of amplifying the marker sequence. A “marker probe” is a nucleic acid sequence or molecule that can be used to identify the presence of a marker locus, e.g., a nucleic acid probe that is complementary to a marker locus sequence. Alternatively, in some aspects, a marker probe refers to a probe of any type that is able to distinguish (i.e., genotype) the particular allele that is present at a marker locus. A “marker locus” is a locus that can be used to track the presence of a second linked locus, e.g., a linked locus that encodes or contributes to expression of a phenotypic trait. For example, a marker locus can be used to monitor segregation of alleles at a locus, such as a QTL, that are genetically or physically linked to the marker locus. Thus, a “marker allele,” alternatively an “allele of a marker locus” is one of a plurality of polymorphic nucleotide sequences found at a marker locus in a population that is polymorphic for the marker locus. Examples of markers include restriction fragment length polymorphism (RFLP) markers, amplified fragment length polymorphism (AFLP) markers, single nucleotide polymorphisms (SNPs), microsatellite markers (e.g. SSRs), sequence-characterized amplified region (SCAR) markers, cleaved amplified polymorphic sequence (CAPS) markers or isozyme markers or combinations of the markers described herein which defines a specific genetic and chromosomal location.

The term “marker assisted selection” refers to the diagnostic process of identifying, optionally followed by selecting a plant from a group of plants using the presence of a molecular marker as the diagnostic characteristic or selection criterion. The process usually involves detecting the presence of a certain nucleic acid sequence or polymorphism in the genome of a plant.

The term “nucleotide” refers to an organic molecule that serves as a monomeric unit of DNA and RNA. The nucleotide position is the position along a chromosome wherein any particular monomeric unit of DNA or RNA is positioned relative to the other monomeric units of DNA or RNA.

The term “probe,” “nucleic acid probe,” or “oligonucleotide probe” as used herein, is one or more synthetic nucleic acid molecules that are complementary to a nucleic acid sequence of interest (target sequence), and hybridize to the sequence of interest when under hybridization conditions. Probes can be used to detect, analyze, and/or visualize the nucleic acid sequence of interest on a molecular level. Specific hybridization of a probe to a nucleic acid sequence of interest can be detected, for example, through a label on the probe. Probes have a length suitable to achieve a desired specificity to the target sequence, however, are generally at least 10 nucleotides long, for example, at least 15 nucleotides, at least 20 nucleotides, or at least 50 nucleotides long. Probes can be immobilized on a solid surface (e.g., nitrocellulose, glass, quartz, fused silica slides), as in an array. The precise sequence of the particular probes described herein can be modified to a certain degree to produce probes that are “substantially identical” to the disclosed probes, but retain the ability to specifically bind to (i.e., hybridize specifically to) the same targets as the probe from which they were derived (see discussion above). Such modifications are specifically covered by reference to the individual probes described herein.

The term “offspring” refers to any plant resulting as progeny from a vegetative or sexual reproduction from one or more parent plants or descendants thereof. For instance an offspring plant may be obtained by cloning or selfing of a parent plant or by crossing two parent plants and includes selfings as well as the F1 or F2 or still further generations. An F1 is a first-generation offspring produced from parents at least one of which is used for the first time as donor of a trait, while offspring of second generation (F2) or subsequent generations (F3, F4, etc.) are specimens produced from selfings of F1's, F2's etc. An F1 may thus be (and usually is) a hybrid resulting from a cross between two true breeding parents (true-breeding is homozygous for a trait), while an F2 may be (and usually is) an offspring resulting from self-pollination of said F1 hybrids.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The terms “percent sequence identity” or “percent identity” or “identity” are used interchangeably to refer to a sequence comparison based on identical matches between correspondingly identical positions in the sequences being compared between two or more amino acid or nucleotide sequences. The percent identity refers to the extent to which two optimally aligned polynucleotide or peptide sequences are invariant throughout a window of alignment of components, e.g., nucleotides or amino acids. Hybridization experiments and mathematical algorithms known in the art may be used to determine percent identity. Many mathematical algorithms exist as sequence alignment computer programs known in the art that calculate percent identity. These programs may be categorized as either global sequence alignment programs or local sequence alignment programs.

The term “plant” refers to a whole plant and any descendant, cell, tissue, or part of a plant. A class of plant that can be used in the present disclosure is generally as broad as the class of higher and lower plants amenable to mutagenesis including angiosperms (monocotyledonous and dicotyledonous plants), gymnosperms, ferns and multicellular algae. Thus, “plant” includes dicot and monocot plants. The term “plant parts” include any part(s) of a plant, including, for example and without limitation: seed (including mature seed and immature seed); a plant cutting; a plant cell; a plant cell culture; a plant organ (e.g., pollen, embryos, flowers, fruits, shoots, leaves, roots, stems, and explants). Plant tissue refers to any tissue of a plant, including but not limited to, tissue from an embryo, shoot, root, stem, seed, stipule, leaf, petal, flower bud, flower, ovule, bract, trichome, branch, petiole, internode, bark, pubescence, tiller, rhizome, frond, blade, ovule, pollen, stamen. A plant tissue or plant organ may be a seed, protoplast, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the plant. In contrast, some plant cells are not capable of being regenerated to produce plants. Regenerable cells in a plant cell or tissue culture may be embryos, protoplasts, meristematic cells, callus, pollen, leaves, anthers, roots, root tips, silk, flowers, kernels, cars, cobs, husks, or stalks. Plant parts include harvestable parts and parts useful for propagation of progeny plants. Plant parts useful for propagation include, for example and without limitation: seed; fruit; a cutting; a seedling; a tuber; and a rootstock. A harvestable part of a plant may be any useful part of a plant, including, for example and without limitation: flower; pollen; seedling; tuber; leaf; stem; fruit; seed; and root. A plant cell is the structural and physiological unit of the plant, comprising a protoplast and a cell wall. A plant cell may be in the form of an isolated single cell, or an aggregate of cells (e.g., a friable callus and a cultured cell), and may be part of a higher organized unit (e.g., a plant tissue, plant organ, and plant). Thus, a plant cell may be a protoplast, a gamete producing cell, or a cell or collection of cells that can regenerate into a whole plant. As such, a seed, which comprises multiple plant cells and is capable of regenerating into a whole plant, is considered a “plant cell.” Described herein are plants in the genus of Cannabis and plants derived thereof, which can be produced asexual or sexual reproduction. The terms “polynucleotide,” “polynucleotide sequence,” “nucleotide,” “nucleotide sequence,” “nucleic acid sequence,” “nucleic acid fragment,” and “isolated nucleic acid fragment” are used interchangeably herein. These terms encompass nucleotide sequences and the like. A polynucleotide may be a polymer of RNA or DNA that is single- or double-stranded, that optionally contains synthetic, non-natural or altered nucleotide bases. A polynucleotide in the form of a polymer of DNA comprises one or more segments of cDNA, genomic DNA, synthetic DNA, or mixtures thereof. Nucleotides (usually found in their 5′-monophosphate form) are referred to by a single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosinc, and “N” for any nucleotide. An “isolated polynucleotide” refers to a polymer of ribonucleotides (RNA) or deoxyribonucleotides (DNA) that is single- or double-stranded, optionally containing synthetic, non-natural or altered nucleotide bases. An isolated polynucleotide in the form of DNA may be comprised of one or more segments of cDNA, genomic DNA or synthetic DNA.

The terms “PCR” or “Polymerase Chain Reaction” refers to a technique for the synthesis of large quantities of specific DNA segments, consisting of a series of repetitive cycles (Perkin Elmer Cetus Instruments, Norwalk, Conn.). Typically, the double stranded DNA is heat denatured, the two primers complementary to the 3′ boundaries of the target segment are annealed at low temperature and then extended at an intermediate temperature. One set of these three consecutive steps comprises a cycle.

The term “polymorphism” refers to a difference in the nucleotide or amino acid sequence of a given region as compared to a nucleotide or amino acid sequence in a homologous-region of another individual, in particular, a difference in the nucleotide of amino acid sequence of a given region which differs between individuals of the same species. A polymorphism is generally defined in relation to a reference sequence. Polymorphisms include single nucleotide differences, differences in sequence of more than one nucleotide, and single or multiple nucleotide insertions, inversions and deletions; as well as single amino acid differences, differences in sequence of more than one amino acid, and single or multiple amino acid insertions, inversions, and deletions.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirements of the application. For example, in diagnostic applications, the oligonucleotide primer is typically 15-25 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

The term “progeny” refers to any subsequent generation of a plant. Progeny is measured using the following nomenclature: F1 refers to the first generation progeny, F2 refers to the second generation progeny, F3 refers to the third generation progeny, and so on.

The term “promoter” refers to a nucleic acid fragment capable of controlling transcription of another nucleic acid fragment. A promoter is capable of controlling the expression of a coding sequence or functional RNA. Functional RNA includes, but is not limited to, transfer RNA (tRNA) and ribosomal RNA (rRNA). The promoter sequence consists of proximal and more distal upstream elements, the latter elements often referred to as enhancers. Accordingly, an “enhancer” is a DNA sequence that can stimulate promoter activity, and may be an innate element of the promoter or a heterologous element inserted to enhance the level or tissue-specificity of a promoter. Promoters may be derived in their entirety from a native gene, or be composed of different elements derived from different promoters found in nature, or even comprise synthetic DNA segments. It is understood that different promoters may direct the expression of a gene in different tissues or cell types, or at different stages of development, or in response to different environmental conditions. New promoters of various types useful in plant cells are constantly being discovered; numerous examples may be found in the compilation by Okamuro and Goldberg (Biochemistry of Plants 15:1-82 (1989)). It is further recognized that since in most cases the exact boundaries of regulatory sequences have not been completely defined, DNA fragments of some variation may have identical promoter activity.

The term “protein” refers to amino acid polymers that contain at least five constituent amino acids that are covalently joined by peptide bonds. The constituent amino acids can be from the group of amino acids that are encoded by the genetic code, which include: alanine, valine, leucine, isoleucine, methionine, phenylalanine, tyrosine, tryptophan, serine, threonine, asparagine, glutamine, cysteine, glycine, proline, arginine, histidine, lysine, aspartic acid, and glutamic acid. As used herein, the term “protein” is synonymous with the related terms “peptide” and “polypeptide.”

The term “purified” as used herein relates to the isolation of a molecule or compound in a form that is substantially free of contaminants normally associated with the molecule or compound in a native or natural environment, or substantially enriched in concentration relative to other compounds present when the compound is first formed, and means having been increased in purity as a result of being separated from other components of the original composition. The term “purified nucleic acid” is used herein to describe a nucleic acid sequence which has been separated, produced apart from, or purified away from other biological compounds including, but not limited to polypeptides, lipids and carbohydrates, while effecting a chemical or functional change in the component (e.g., a nucleic acid may be purified from a chromosome by removing protein contaminants and breaking chemical bonds connecting the nucleic acid to the remaining DNA in the chromosome).

The term “quantitative trait loci” or “QTL” refers to the genetic elements controlling a quantitative trait.

The term “reference plant” or “reference genome” refers to a wild-type or reference sequence that SNPs or other markers in a test sample can be compared to in order to detect a modification of the sequence in the test sample.

The phrase “resistance to hermaphroditism” or “hermaphroditism resistance” as used herein refers to the ability to inhibit or suppress the occurrence of hermaphroditism.

The term “RNA transcript” refers to a product resulting from RNA polymerase-catalyzed transcription of a DNA sequence. When an RNA transcript is a perfect complementary copy of a DNA sequence, it is referred to as a primary transcript or it may be a RNA sequence derived from post-transcriptional processing of a primary transcript and is referred to as a mature RNA. “Messenger RNA” (“mRNA”) refers to RNA that is without introns and that can be translated into protein by the cell. “cDNA” refers to a DNA that is complementary to and synthesized from an mRNA template using the enzyme reverse transcriptase. The cDNA can be single-stranded or converted into the double-stranded by using the Klenow fragment of DNA polymerase I. “Sense” RNA refers to RNA transcript that includes mRNA and so can be translated into protein within a cell or in vitro. “Antisense RNA” refers to a RNA transcript that is complementary to all or part of a target primary transcript or mRNA and that blocks expression or transcripts accumulation of a target gene (U.S. Pat. No. 5,107,065). The complementarity of an antisense RNA may be with any part of the specific gene transcript, i.e. at the 5′ non-coding sequence, 3′ non-coding sequence, introns, or the coding sequence. “Functional RNA” refers to antisense RNA, ribozyme RNA, or other RNA that may not be translated but yet has an effect on cellular processes.

The terms “similar,” “substantially similar” and “corresponding substantially” as used herein refer to nucleic acid fragments wherein changes in one or more nucleotide bases do not affect the ability of the nucleic acid fragment to mediate gene expression or produce a certain phenotype. These terms also refer to modifications of nucleic acid fragments, such as deletion or insertion of one or more nucleotides that do not substantially alter the functional properties of the resulting nucleic acid fragment relative to the initial, unmodified fragment. It is therefore understood that the present disclosure encompasses more than the specific exemplary sequences. A “substantially homologous sequence” refers to variants of the disclosed sequences such as those that result from site-directed mutagenesis, as well as synthetically derived sequences. A substantially homologous sequence also refers to fragments of a particular promoter nucleotide sequence disclosed herein that operate to promote the constitutive expression of an operably linked heterologous nucleic acid fragment. These promoter fragments will comprise at least about 20 contiguous nucleotides, preferably at least about 50 contiguous nucleotides, more preferably at least about 75 contiguous nucleotides, even more preferably at least about 100 contiguous nucleotides of the particular promoter nucleotide sequence disclosed herein. The nucleotides of such fragments will usually comprise the TATA recognition sequence of the particular promoter sequence. Such fragments may be obtained by use of restriction enzymes to cleave the naturally occurring promoter nucleotide sequences disclosed herein; by synthesizing a nucleotide sequence from the naturally occurring promoter DNA sequence; or may be obtained through the use of PCR technology. See particularly, Mullis et al., Methods Enzymol. 155:335-350 (1987), and Higuchi, R. In PCR Technology: Principles and Applications for DNA Amplifications; Erlich, H. A., Ed.; Stockton Press Inc.: New York, 1989. Again, variants of these promoter fragments, such as those resulting from site-directed mutagenesis, are encompassed by the present disclosure.

The term “single nucleotide polymorphism (SNP)” refers to a change in which a single base in the DNA differs from the usual base at that position. These single base changes are called SNPs or “snips.”

The term “target region” or “nucleic acid target” refers to a nucleotide sequence that resides at a specific chromosomal location. The “target region” or “nucleic acid target” is specifically recognized by a probe.

The term “transition” as used herein refers to the transition of a nucleotide at any specific genomic position with that of a different nucleotide.

The term “transgenic” refers to any cell, cell line, callus, tissue, plant part or plant, the genome of which has been altered by the presence of a heterologous nucleic acid, such as a recombinant DNA construct, including those initial transgenic events as well as those created by sexual crosses or asexual propagation from the initial transgenic event. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation. The term “transgenic plant” refers to a plant which comprises within its genome a heterologous polynucleotide. For example, the heterologous polynucleotide is stably integrated within the genome such that the polynucleotide is passed on to successive generations. The heterologous polynucleotide may be integrated into the genome alone or as part of a recombinant DNA construct. A “transgene” is a gene that has been introduced into the genome by a transformation procedure.

The term “translation leader sequence” refers to a polynucleotide sequence located between the promoter sequence of a gene and the coding sequence. The translation leader sequence is present in the fully processed mRNA upstream of the translation start sequence. The translation leader sequence may affect processing of the primary transcript to mRNA, mRNA stability or translation efficiency. Examples of translation leader sequences have been described (Turner, R. and Foster, G. D., Molecular Biotechnology 3:225 (1995)).

The term “variety” as used herein has identical meaning to the corresponding definition in the International Convention for the Protection of New Varieties of Plants (UPOV treaty), of Dec. 2, 1961, as Revised at Geneva on Nov. 10, 1972, on Oct. 23, 1978, and on Mar. 19, 1991. Thus, “variety” means a plant grouping within a single botanical taxon of the lowest known rank, which grouping, irrespective of whether the conditions for the grant of a breeder's right are fully met, can be i) defined by the expression of the characteristics resulting from a given genotype or combination of genotypes, ii) distinguished from any other plant grouping by the expression of at least one of the said characteristics and iii) considered as a unit with regard to its suitability for being propagated unchanged.

### Cannabis

Cannabis has long been used for drug and industrial purposes, fiber (hemp), for seed and seed oils, for medicinal purposes, and for recreational purposes. Industrial hemp products are made from Cannabis plants selected to produce an abundance of fiber. Some Cannabis varieties have been bred to produce minimal levels of THC, the principal psychoactive constituent responsible for the psychoactivity associated with marijuana. Marijuana has historically consisted of the dried flowers of Cannabis plants selectively bred to produce high levels of THC and other psychoactive cannabinoids. Various extracts including hashish and hash oil are also produced from the plant.

Cannabis is an annual, dioecions, flowering herb. The leaves are palmately compound or digitate, with serrate leaflets. Cannabis normally has imperfect flowers, with staminate “male” and pistillate “female” flowers occurring on separate plants. It is not unusual, however, for individual plants to separately bear both male and female flowers (i.e., have monoecious plants). Although monoecious plants are often referred to as “hermaphrodites,” true hermaphrodites (which are less common in Cannabis) bear staminate and pistillate structures on individual flowers, whereas monoecious plants bear male and female flowers at different locations on the same plant.

The life cycle of Cannabis varies with each variety but can be generally summarized into germination, vegetative growth, and reproductive stages. Because of heavy breeding and selection by humans, most Cannabis seeds have lost dormancy mechanisms and do not require any pre-treatments or winterization to induce germination (See, Clarke, R C et al. “Cannabis: Evolution and Ethnobotany” University of California Press 2013). Seeds placed in viable growth conditions are expected to germinate in about 3 to 7 days. The first true leaves of a Cannabis plant contain a single leaflet, with subsequent leaves developing in opposite formation with increasing number of leaflets. Leaflets can be narrow or broad depending on the morphology of the plant grown. Cannabis plants are normally allowed to grow vegetatively for the first 4 to 8 weeks. During this period, the plant responds to increasing light with faster and faster growth. Under ideal conditions, Cannabis plants can grow up to 2.5 inches a day and are capable of reaching heights of up to 20 feet. Indoor growth pruning techniques tend to limit Cannabis size through careful pruning of apical or side shoots.

Cannabis is diploid, having a chromosome complement of 2n=20, although polyploid individuals have been artificially produced. The first genome sequence of Cannabis, which is estimated to be 820 Mb in size, was published in 2011 by a team of Canadian scientists (Bakel et al, “The draft genome and transcriptome of Cannabis sativa” Genome Biology 12:R102).

All known varieties of Cannabis are wind-pollinated and the fruit is an achene. Most varieties of Cannabis are short day plants, with the possible exception of C. sativa subsp. sativa var. spontanca (=C. ruderalis), which is commonly described as “auto-flowering” and may be day-neutral.

The genus Cannabis was formerly placed in the Nettle (Urticaceae) or Mulberry (Moraceae) family, and later, along with the Humulus genus (hops), in a separate family, the Hemp family (Cannabaceae sensu stricto). Recent phylogenetic studies based on cpDNA restriction site analysis and gene sequencing strongly suggest that the Cannabaceae sensu stricto arose from within the former Celtidaceae family, and that the two families should be merged to form a single monophyletic family, the Cannabaceae sensu lato.

Cannabis plants produce a unique family of terpeno-phenolic compounds called cannabinoids. Cannabinoids, terpenoids, and other compounds are secreted by glandular trichomes that occur most abundantly on the floral calyxes and bracts of female plants. As a drug it usually comes in the form of dried flower buds (marijuana), resin (hashish), or various extracts collectively known as hashish oil. There are at least 483 identifiable chemical constituents known to exist in the Cannabis plant (Rudolf Brenneisen, 2007, Chemistry and Analysis of Phytocannabinoids (cannabinoids produced produced by Cannabis) and other Cannabis Constituents, In Marijuana and the Cannabinoids, ElSohly, ed.; incorporated herein by reference) and at least 85 different cannabinoids have been isolated from the plant (El-Alfy, Abir T, et al., 2010, “Antidepressant-like effect of delta-9-tetrahydrocannabinol and other cannabinoids isolated from Cannabis sativa L”, Pharmacology Biochemistry and Behavior 95(4): 434-42; incorporated herein by reference). The two cannabinoids usually produced in greatest abundance are cannabidiol (CBD) and/or Δ9-tetrahydrocannabinol (THC). THC is psychoactive while CBD is not. See, ElSohly, ed. (Marijuana and the Cannabinoids, Humana Press Inc., 321 papers, 2007), which is incorporated herein by reference in its entirety, for a detailed description and literature review on the cannabinoids found in marijuana.

Cannabinoids are the most studied group of secondary metabolites in Cannabis. Most exist in two forms, as acids and in neutral (decarboxylated) forms. The acid form is designated by an “A” at the end of its acronym (i.e. THCA). The phytocannabinoids are synthesized in the plant as acid forms, and while some decarboxylation docs occur in the plant, it increases significantly post-harvest and the kinetics increase at high temperatures. (Sanchez and Verpoorte 2008). The biologically active forms for human consumption are the neutral forms. Decarboxylation is usually achieved by thorough drying of the plant material followed by heating it, often by either combustion, vaporization, or heating or baking in an oven. Unless otherwise noted, references to cannabinoids in a plant include both the acidic and decarboxylated versions (e.g., CBD and CBDA).

Detection of neutral and acidic forms of cannabinoids are dependent on the detection method utilized. Two popular detection methods are high-performance liquid chromatography (HPLC) and gas chromatography (GC). HPLC separates, identifies, and quantifies different components in a mixture, and passes a pressurized liquid solvent containing the sample mixture through a column filled with a solid adsorbent material. Each molecular component in a sample mixture interacts differentially with the adsorbent material, thus causing different flow rates for the different components and therefore leading to separation of the components. In contrast, GC separates components of a sample through vaporization. The vaporization required for such separation occurs at high temperature. Thus, the main difference between GC and HPLC is that GC involves thermal stress and mainly resolves analytes by boiling points while HPLC does not involve heat and mainly resolves analytes by polarity. The consequence of utilizing different methods for cannabinoid detection therefore is that HPLC is more likely to detect acidic cannabinoid precursors, whereas GC is more likely to detect decarboxylated neutral cannabinoids.

The cannabinoids in cannabis plants include, but are not limited to, Δ9-Tetrahydrocannabinol (Δ9-THC), Δ8-Tetrahydrocannabinol (Δ8-THC), Cannabichromene (CBC), Cannabicyclol (CBL), Cannabidiol (CBD), Cannabiclsoin (CBE), Cannabigerol (CBG), Cannabinidiol (CBND), Cannabinol (CBN), Cannabitriol (CBT), and their propyl homologs, including, but are not limited to cannabidivarin (CBDV), Δ9-Tetrahydrocannabivarin (THCV), cannabichromevarin (CBCV), and cannabigerovarin (CBGV). See, Holley et al. (Constituents of Cannabis sativa L. XI Cannabidiol and cannabichromene in samples of known geographical origin, J. Pharm. Sci. 64:892-894, 1975) and De Zeeuw et al. (Cannabinoids with a propyl side chain in Cannabis, Occurrence and chromatographic behavior, Science 175:778-779), each of which is herein incorporated by reference in its entirety for all purposes. Non-THC cannabinoids can be collectively referred to as “CBs”, wherein CBs can be one of THCV, CBDV, CBGV, CBCV, CBD, CBC, CBE, CBG, CBN, CBND, and CBT cannabinoids.

### Hermaphroditism Markers and Haplotypes

The present disclosure describes the discovery of novel markers indicating resistance to hermaphroditism. The disclosure includes methods of identifying or selecting plants resistant to hermaphroditism, which in some implementations include i) obtaining nucleic acids from a sample plant or its germplasm; (ii) detecting one or more markers that indicate resistance to hermaphroditism, and (iii) indicating resistance to hermaphroditism. In some examples, the method further includes identifying one of more plants resistant to hermaphroditism, and/or selecting the one or more plants indicating resistance to hermaphroditism.

In an aspect, the markers described herein comprise polymorphisms relative to the Abacus Cannabis reference genome Csat_AbacusV2; NCBI assembly accession GCA_025232715.1 (CsaAba2). Exemplary markers are described in Tables 2, 5, and 7, which identify polymorphisms that indicate resistance to hermaphroditism, positioning on their respective chromosomes, reference and alternative calls, as well as assigning sequence identifiers. Table 8 further describes the beneficial genotype with respect to the described markers. A subset of three hermaphroditism SNP marker genotypes that are predictive of female flowering are described in Table 9.

The markers of the present disclosure may be described in numerous fashions. To illustrate, for non-limiting exemplary purposes, marker 142494_1054190 as described in Table 2 is described as being positioned at base pair (bp) position 5,705,332 on chromosome X of Csat_AbacusV2; NCBI assembly accession GCA_025232715.1 (CsaAba2) reference genome. Likewise, marker 142494_1054190 is described as being positioned at nucleotide 26 of SEQ ID NO:1 or position 51 of SEQ ID NO: 62. In another non-limiting example, marker 142494_1081185 is described as positioned at bp 5,732,323 on chromosome X, position 26 of SEQ ID NO: 2, and position 51 of SEQ ID NO: 63. In another non-limiting example, marker Cannabis.v1_scf2268-48774_101 is described as positioned at bp 17,971,672 on chromosome X, position 26 of SEQ ID NO: 14, and position 51 of SEQ ID NO: 75. In a further non-limiting example, marker 142169_2381612 is described as positioned at bp 14,810,444 on chromosome 3, position 26 of SEQ ID NO: 15, and position 51 of SEQ ID NO: 76.

The present disclosure further describes the discovery of novel haplotype markers for plants, including cannabis. Haplotypes refer to the genotype of a plant at a plurality of genetic loci, e.g., a combination of alleles or markers. Haplotypes can refer to sequence polymorphisms at a particular locus, such as a single marker locus, or sequence polymorphisms at multiple loci along a chromosomal segment in a given genome. Markers of the present disclosure and within the haplotypes described are significantly correlated to plants having resistance to hermaphroditism, which thus can be used to screen plants exhibiting resistance to hermaphroditism.

Tables 2, 5, and 7 further describe markers within a haplotype that identify polymorphisms that confer resistance to hermaphroditism, which describe the haplotype both with respect to the left and right flanking markers, and with respect to the left and right flanking positioning on their respective chromosomes. To illustrate, for non-limiting exemplary purposes, marker 142494_1054190 is within a haplotype defined as being between left flanking marker 142494_1045160 at position 5,696,400 on chromosome X and right flanking marker 141494_1063195 at position 5,714,336 on chromosome X. Further non-limiting examples include marker 142494_1081185, which is within a haplotype defined as being between positions 5,725,231 and 5,737,968 on chromosome X; marker Cannabis.v1_scf2268-48774_101, which is within a haplotype defined as being between positions 17,900,648 and 17,980,443 on chromosome X; and marker 142169_2381612, which is within a haplotype defined as being between positions 14,797,288 and 14,824,861 on chromosome 3.

### Quantitative Trait Loci

The term chromosome interval designates a contiguous linear span of genomic DNA that resides on a single chromosome. A chromosome interval may comprise a quantitative trait locus (“QTL”) linked with a genetic trait and the QTL may comprise a single gene or multiple genes associated with the genetic trait. The boundaries of a chromosome interval comprising a QTL are drawn such that a marker that lies within the chromosome interval can be used as a marker for the genetic trait, as well as markers genetically linked thereto. Each interval comprising a QTL comprises at least one gene conferring a given trait, however knowledge of how many genes are in a particular interval is not necessary to make or practice the compositions or methods of the present disclosure, as such an interval will segregate at meiosis as a linkage block. Accordingly, a chromosomal interval comprising a QTL may therefore be readily introgressed and tracked in a given genetic background using the methods and compositions provided herein.

Identification of chromosomal intervals and QTL is therefore beneficial for detecting and tracking a genetic trait, such as resistance to hermaphroditism, in plant populations. In some of the methods disclosed herein, this is accomplished by identification of markers linked to a particular QTL. The principles of QTL analysis and statistical methods for calculating linkage between markers and useful QTL include penalized regression analysis, ridge regression, single point marker analysis, complex pedigree analysis, Bayesian MCMC, identity-by-descent analysis, interval mapping, composite interval mapping (CIM), and Haseman-Elston regression. QTL analyses may be performed with the help of a computer and specialized software available from a variety of public and commercial sources known to those of skill in the art.

### Detection of Markers

The present disclosure describes the use of detecting markers associated with hermaphroditism resistance. Marker detection is well known in the art. For example, amplification of a target polynucleotide (e.g., by PCR) using a particular amplification primer pair that permit the primer pair to hybridize to the target polynucleotide to which a primer having the corresponding sequence (or its complement) would bind and preferably to produce an identifiable amplification product (the amplicon) having a marker is well known in the art.

Methods for designing PCR primers and PCR cloning are generally known in the art and are disclosed, for example, in Sambrook et al. (2014) Molecular Cloning: A Laboratory Manual (Fourth Edition, Cold Spring Harbor Laboratory Press, Plainview, N.Y.). See also, Innis and Gelfand, eds. (1999) PCR Methods Manual (Academic Press, New York). Methods of amplification are further described in U.S. Pat. Nos. 4,683,195, 4,683,202. These methods as well as other methods known in the art of DNA amplification may be used. It will be appreciated that suitable primers can be designed using any suitable method. It is not intended that the disclosed compositions or methods be limited to any particular primer or primer pair. It is not intended that the primers be limited to generating an amplicon of any particular size. For example, the primers used to amplify the marker loci and alleles herein are not limited to amplifying the entire region of the relevant locus. The primers can generate an amplicon of any suitable length that is longer or shorter than those disclosed herein. In some examples, marker amplification produces an amplicon that is at least 20 nucleotides in length, or alternatively, at least 50 nucleotides in length, or alternatively, at least 100 nucleotides in length, or alternatively, at least 200 nucleotides in length. In some examples, marker amplification produces an amplicon that is 20 to 200 nucleotides in length, for example, 20 to 190 nucleotides, 20 to 180 nucleotides, 20 to 170 nucleotides, 20 to 160 nucleotides, 20 to 160 nucleotides, 20 to 150 nucleotides, 20 to 140 nucleotides, 20 to 120 nucleotides, 20 to 110 nucleotides, 20 to 100 nucleotides, 20 to 90 nucleotides, 20 to 80 nucleotides, 20 to 70 nucleotides, 20 to 60 nucleotides, 20 to 55 nucleotides, 20 to 50 nucleotides, 20 to 45 nucleotides, 20 to 40 nucleotides, 20 to 35 nucleotides, 20 to 30 nucleotides, or 20 to 25 nucleotides in length. In some examples, the amplicon is 51 nucleotides in length. In some examples, the amplicon is 101 nucleotides in length. It is understood that a number of parameters in a specific PCR protocol may need to be adjusted to specific laboratory conditions and may be slightly modified and yet allow for the collection of similar results. The primers can be radiolabeled, or labeled by any suitable means (e.g., using a non-radioactive fluorescent tag), to allow for rapid visualization of the different size amplicons following an amplification reaction without any additional labeling step or visualization step. The known nucleic acid sequences for the genes described herein are sufficient to enable one of skill in the art to routinely select primers for amplification of the gene of interest.

Other examples of nucleic acid amplification methods include, but are not limited to, reverse-transcription PCR (RT-PCR), quantitative real-time PCR (qPCR), quantitative real-time reverse transcriptase PCR (qRT-PCR) (see, e.g., Adams, A beginner's guide to RT-PCR, qPCR and RT-qPCR, Biochemist (Lond) (2020) 42(3): 48-53), isothermal amplification methods (see, e.g., Zanoli et al., Biosensors (2013) 3(1): 18-43), nucleic acid sequence-based amplification (NASBA) (see, e.g., Deiman and Sillekens, Mol Biotechnol (2002) 20(2): 163-79), loop-mediated isothermal amplification (LAMP) (see, e.g., Notomi et al., (2000) Nucleic Acids Res. 28(12): e63), helicase-dependent amplification (HDA) (see, e.g., Cao et al., Helicase-dependent amplification of nucleic acids, Curr Protoc Mol Biol, 104:15.11.1-15.11.12, 2013), rolling circle amplification (RCA) (see, e.g, Yao et al. Nature Protocols (2021) 16, 5460-5483), multiple displacement amplification (MDA) (see, e.g, Spits et al. Nature Protocols (2006) 1:1965-1970), recombinase polymerase amplification (RPA) (see, e.g., Lobato et al., Trends Analyt Chem (2018) 98:19-35), ligase chain reaction (LCR) (see e.g., Gibriel and Adel, Mutat Res Rev Mutat Res. (2017) 773:66-90), transcription amplification (see e.g., Kwoh et al. (1989) Proc. Natl. Acad. Sci. USA 86:1173), self-sustained sequence replication (see e.g., Guatelli et al. (1990) Proc. Natl. Acad. Sci. USA 87:1874), dot PCR, and linker adapter PCR. Suitable amplification methods are also described, for example, in Sambrook et al. (2014) Molecular Cloning: A Laboratory Manual (Fourth Edition, Cold Spring Harbor Laboratory Press, Plainview, N.Y.).

An amplicon is an amplified nucleic acid, e.g., a nucleic acid that is produced by amplifying a template nucleic acid by any available amplification method (e.g., PCR, LCR, transcription, or the like). A genomic nucleic acid is a nucleic acid that corresponds in sequence to a heritable nucleic acid in a cell. Common examples include nuclear genomic DNA and amplicons thereof. A genomic nucleic acid is, in some cases, different from a spliced RNA, or a corresponding cDNA, in that the spliced RNA or cDNA is processed, e.g., by the splicing machinery, to remove introns. Genomic nucleic acids optionally comprise non-transcribed (e.g., chromosome structural sequences, promoter regions, enhancer regions, etc.) and/or non-translated sequences (e.g., introns), whereas spliced RNA/cDNA typically do not have non-transcribed sequences or introns. A template nucleic acid is a nucleic acid that serves as a template in an amplification reaction (e.g., a polymerase based amplification reaction such as PCR, a ligase mediated amplification reaction such as LCR, a transcription reaction, or the like). A template nucleic acid can be genomic in origin, or alternatively, can be derived from expressed sequences, e.g., a cDNA or an EST. Details regarding the use of these and other amplification methods can be found in any of a variety of standard texts. Many available biology texts also have extended discussions regarding PCR and related amplification methods and one of skill will appreciate that essentially any RNA can be converted into a double stranded DNA suitable for restriction digestion, PCR expansion and sequencing using reverse transcriptase and a polymerase.

PCR detection and quantification using dual-labeled fluorogenic oligonucleotide probes, commonly referred to as “TaqMan™” probes, can also be performed according to the present disclosure. These probes are composed of short (e.g., 20-25 base) oligodeoxynucleotides that are labeled with two different fluorescent dyes. On the 5′ terminus of each probe is a reporter dye, and on the 3′ terminus of each probe a quenching dye is found. The oligonucleotide probe sequence is complementary to an internal target sequence present in a PCR amplicon. When the probe is intact, energy transfer occurs between the two fluorophores and emission from the reporter is quenched by the quencher by FRET. During the extension phase of PCR, the probe is cleaved by 5′ nuclease activity of the polymerase used in the reaction, thereby releasing the reporter from the oligonucleotide-quencher and producing an increase in reporter emission intensity. TaqMan™ probes are oligonucleotides that have a label and a quencher, where the label is released during amplification by the exonuclease action of the polymerase used in amplification, providing a real time measure of amplification during synthesis. A variety of TaqMan™ reagents are commercially available, e.g., from Applied Biosystems as well as from a variety of specialty vendors such as Biosearch Technologies.

In general, synthetic methods for making oligonucleotides, including probes, primers, molecular beacons, PNAs, LNAs (locked nucleic acids), etc., are well known. For example, oligonucleotides can be synthesized chemically according to the solid phase phosphoramidite triester method described. Oligonucleotides, including modified oligonucleotides, can also be ordered from a variety of commercial sources.

Nucleic acid probes to the marker loci can be cloned and/or synthesized. Any suitable label can be used with a probe. Detectable labels suitable for use with nucleic acid probes include, for example, any composition detectable by spectroscopic, radioisotopic, photochemical, biochemical, immunochemical, electrical, optical or chemical means. Useful labels include biotin for staining with labeled streptavidin conjugate, magnetic beads, fluorescent dyes, radio labels, enzymes, and colorimetric labels. Other labels include ligands which bind to antibodies labeled with fluorophores, chemiluminescent agents, and enzymes. A probe can also constitute radio labeled PCR primers that are used to generate a radio labeled amplicon. It is not intended that the nucleic acid probes be limited to any particular size.

Amplification is not always a requirement for marker detection (e.g. Southern blotting and RFLP detection). Separate detection probes can also be omitted in amplification/detection methods, e.g., by performing a real time amplification reaction that detects product formation by modification of the relevant amplification primer upon incorporation into a product, incorporation of labeled nucleotides into an amplicon, or by monitoring changes in molecular rotation properties of amplicons as compared to unamplified precursors (e.g., by fluorescence polarization).

Genetic markers can be detected by sequencing a nucleic acid fragment comprising a genetic marker of interest, or by whole genome sequencing. Non-limiting examples of suitable sequencing methods include capillary electrophoresis (e.g., Sanger sequencing) and high-throughput sequencing (e.g., Illumina® or 454 Sequencing®). High-throughput sequencing includes short read or long read techniques. In some implementations, sequencing includes whole genome sequencing (e.g., sequencing the genome of a cannabis plant of interest). In some implementations, sequencing includes targeted sequencing (sequencing of a particular nucleic acid or amplicon of interest). In some implementations, sequencing includes sequencing a transcriptome (RNA-Seq) (e.g., sequencing the transcriptome of a cannabis plant of interest). In some implementations, sequencing does not include RNA-Seq.

### Hermaphroditism Resistance Genes

Candidate genes conferring resistance to hermaphroditism are provided herein. Candidate genes for hermaphroditism resistance may include, but are not limited to, a calcium/calcium/calmodulin-dependent Serine/Threonine-kinase; Probable elongation factor 1-gamma 2; SWI/SNF complex subunit SWI3C; a DPP6 N-terminal domain-like protein; DELLA up-regulated gene; NAD KINASE 2; Ubinuclein-1; a putative reverse transcriptase (At2g05200); Cupredoxin superfamily protein; protein phosphatase 2C 15; Cyclin-A2-2/Cyclin-A2-3; Serine/threonine protein phosphatase 2A 57 kDa regulatory subunit B′ kappa isoform/2A 59 kDa regulatory subunit B′ gamma isoform/2A 59 kDa regulatory subunit B′ zeta isoform/2A 57 kDa regulatory subunit B′ beta isoform; Enhancer of polycomb-like transcription factor protein; a serine-rich protein-like protein (AT5G25280); a TTF-type zinc finger protein with HAT dimerization domain-containing protein; Catalase-1/2/3; 1-aminocyclopropane-1-carboxylate synthase 2/5/7/8/9/10/11/12; ACS2/5/7/8/9/10/11/12; At1g01480/At5g65800/At4g26200/At4g37770/At3g49700/At1g62960/At4g08040/At5g51690; E3 ubiquitin-protein ligase RZFP34; Alpha carbonic anhydrase; Plant calmodulin-binding protein-like protein; Galactose oxidase/kelch repeat superfamily protein; Lipid phosphate phosphatase epsilon 1/2, chloroplastic; Mannan endo-1,4-beta-mannosidase; or 1-aminocyclopropane-1-carboxylate synthase. In some examples, the candidate genes are one or more of SWI/SNF complex subunit SWI3C (SWI3C); catalase 2 (CAT2), or 1-aminocyclopropane-1-carboxylate synthase 12 (ACS12).

Also provided are genes conferring resistance to hermaphroditism, or associated with resistance to hermaphroditism. In some examples, genes conferring resistance to hermaphroditism, or associated with resistance to hermaphroditism, include one or more of: SWI/SNF complex subunit SWI3C (SWI3C); catalase 2 (CAT2), or 1-aminocyclopropane-1-carboxylate synthase 12 (ACS12). In a non-limiting example, the genes associated with resistance to hermaphroditism include SWI3C. In another example, the genes conferring resistance to hermaphroditism, or associated with resistance to hermaphroditism, include CAT2. In a further example, the genes conferring resistance to hermaphroditism, or associated with resistance to hermaphroditism, include ACS12. In some examples, the genes conferring resistance to hermaphroditism, or associated with resistance to hermaphroditism, include at least two of: SWI3C, CAT2, and ACS12. In some examples, the genes conferring resistance to hermaphroditism, or associated with resistance to hermaphroditism, include SWI3C, CAT2, and ACS12. In some examples, the genes conferring resistance to hermaphroditism, or associated with resistance to hermaphroditism, consist of, or essentially consist of: SWI3C, CAT2, and ACS12.

Genes conferring resistance to hermaphroditism can be used to identify or select plants that are resistant to hermaphroditism. For example, a method of selecting or identifying a plant resistant to hermaphroditism, as disclosed herein, includes (i) obtaining nucleic acids from a plant or its germplasm; (ii) analyzing the sample to detect one or more nucleic acid polymorphisms in: (a) switch/sucrose nonfermenting 3C (SWI3C), (b) catalase 2 (CAT2), or (c) 1-aminocyclopropane-1-carboxylate synthase 12 (ACS12), thereby identifying and/or selecting a plant having resistance to hermaphroditism. In some examples, the plant having resistance to hermaphroditism is selected. In some examples, a polymorphism is detected in SWI3C. In some examples, a polymorphism is detected in CAT2. In further examples, a polymorphism is detected in ACS12. In some examples, one or more polymorphisms are detected in at least two of SWI3C, CAT2, and ACS12. In some examples, one or more polymorphism are detected in SWI3C, CAT2, and ACS12. In some examples, analyzing the sample includes analyzing at least two of SWI3C, CAT2, and ACS12. In some examples, analyzing the sample includes analyzing SWI3C, CAT2, and ACS12. In some examples, analyzing the sample consists of, or essentially consists of, analyzing SWI3C, CAT2, and ACS12. In some examples, the polymorphism is a beneficial polymorphism (associated with resistance to hermaphroditism). In some examples, the polymorphism increases resistance to hermaphroditism relative to a plant without the polymorphism or relative to a plant with a detrimental polymorphism (a polymorphism associated with hermaphroditism). In some examples, the one or more nucleic acid polymorphisms comprise a substitution corresponding to position 728 bp from the start codon of Abacus SWI3C (728 bp from the start codon in the coding sequence of SWI3C; see, SEQ ID NO: 33), or a substitution corresponding to position 1193 bp from the start codon of Abacus SWI3C (1193 bp from the start codon in the coding sequence of SWI3C; see, SEQ ID NO: 33). In some examples, the one or more nucleic acid polymorphisms comprise a substitution corresponding to position 338 bp from the start of the 3′UTR of Abacus CAT2 (see, SEQ ID NO: 44), or an insertion starting at a position corresponding to 394 bp from the start of the 3′UTR of Abacus CAT2 (see, SEQ ID NO: 44). In some examples, the one or more nucleic acid polymorphisms comprise a deletion corresponding to position 1500 bp from the start codon of Abacus ACS12 (1500 bp from the start codon in the coding sequence of ACS12; see, SEQ ID NO: 50), an insertion corresponding to position 25 bp from the start of the 3′UTR of Abacus ACS12 (see, SEQ ID NO: 58), or a substitution corresponding to position 31 bp from the start of the 3′UTR of Abacus ACS12 (see, SEQ ID NO: 58). In some implementations, the plant identified as having resistance to hermaphroditism is selected for further analysis, propagation, plant breeding, or for making a product. In some implementations, the plant is a cannabis plant.

Preferred substantially similar nucleic acid sequences encompassed by this disclosure are those sequences that are 80% identical to the nucleic acid fragments reported herein or which are 80% identical to any portion of the nucleotide sequences reported herein. More preferred are nucleic acid fragments which are 90% identical to the nucleic acid sequences reported herein, or which are 90% identical to any portion of the nucleotide sequences reported herein. Most preferred are nucleic acid fragments which are 95% identical to the nucleic acid sequences reported herein, or which are 95% identical to any portion of the nucleotide sequences reported herein. It is understood that many levels of sequence identity are useful in identifying related polynucleotide sequences. Useful examples of percent identities are those listed above, or also preferred is any integer percentage from 72% to 100%, such as 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100%.

In some examples, an isolated polynucleotide is provided comprising a nucleotide sequence having at least 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity compared to the claimed sequence, based on the Clustal V method of alignment with pairwise alignment default parameters (KTUPLE=2, GAP PENALTY=5, WINDOW=4 and DIAGONALS SAVED=4).

Local sequence alignment programs are similar in their calculation, but only compare aligned fragments of the sequences rather than utilizing an end-to-end analysis. Local sequence alignment programs such as BLAST can be used to compare specific regions of two sequences. A BLAST comparison of two sequences results in an E-value, or expectation value, that represents the number of different alignments with scores equivalent to or better than the raw alignment score, S, that are expected to occur in a database search by chance. The lower the E value, the more significant the match. Because database size is an element in E-value calculations, E-values obtained by BLASTing against public databases, such as GENBANK, have generally increased over time for any given query/entry match. In setting criteria for confidence of polypeptide function prediction, a “high” BLAST match is considered herein as having an E-value for the top BLAST hit of less than 1E-30; a medium BLASTX E-value is 1E-30 to 1E-8; and a low BLASTX E-value is greater than 1E-8. The protein function assignment in the present disclosure is determined using combinations of E-values, percent identity, query coverage and hit coverage. Query coverage refers to the percent of the query sequence that is represented in the BLAST alignment. Hit coverage refers to the percent of the database entry that is represented in the BLAST alignment. In some examples of the methods disclosed herein, function of a query polypeptide can be inferred from function of a protein homolog where either (1) hit_p<1e-30 or % identity >35% AND query_coverage>50% AND hit_coverage>50%, or (2) hit_p<1e-8 AND query_coverage>70% AND hit_coverage>70%. The following abbreviations are produced during a BLAST analysis of a sequence. SEQ NUM provides the SEQ ID NO for the listed recombinant polynucleotide sequences. CONTIG_ID provides an arbitrary sequence name taken from the name of the clone from which the cDNA sequence was obtained. PROTEIN_NUM provides the SEQ ID NO for the recombinant polypeptide sequence NCBI_GI provides the GenBank ID number for the top BLAST bit for the sequence. The top BLAST hit is indicated by the National Center for Biotechnology Information GenBank Identifier number. NCBI_GI_DESCRIPTION refers to the description of the GenBank top BLAST hit for sequence. E_VALUE provides the expectation value for the top BLAST match. MATCH_LENGTH provides the length of the sequence which is aligned in the top BLAST match TOP_HIT_PCT_IDENT refers to the percentage of identically matched nucleotides (or residues) that exist along the length of that portion of the sequences which is aligned in the top BLAST match. CAT_TYPE indicates the classification scheme used to classify the sequence. GO_BP=Gene Ontology Consortium—biological process; GO_CC=Gene Ontology Consortium—cellular component; GO_MF=Gene Ontology Consortium molecular function; KEGG=KEGG functional hierarchy (KEGG=Kyoto Encyclopedia of Genes and Genomes); EC=Enzyme Classification from ENZYME data bank release 25.0; POI=Pathways of Interest. CAT_DESC provides the classification scheme subcategory to which the query sequence was assigned. PRODUCT_CAT_DESC provides the FunCAT annotation category to which the query sequence was assigned. PRODUCT_HIT_DESC provides the description of the BLAST hit which resulted in assignment of the sequence to the function category provided in the cat_desc column. HIT_E provides the E value for the BLAST hit in the hit_desc column. PCT_IDENT refers to the percentage of identically matched nucleotides (or residues) that exist along the length of that portion of the sequences which is aligned in the BLAST match provided in hit_desc. QRY_RANGE lists the range of the query sequence aligned with the bit. HIT_RANGE lists the range of the hit sequence aligned with the query, provides the percent of query sequence length that matches QRY_CVRG provides the percent of query sequence length that matches to the hit (NCBI) sequence in the BLAST match (% gry cvrg=(match length/query total length)×100). HIT_CVRG provides the percent of hit sequence length that matches to the query sequence in the match generated using BLAST (% hit cvrg=(match lengthy hit total length)×100).

Methods for aligning sequences for comparison are well-known in the art. Various programs and alignment algorithms are described. In an example, percent identity between two polynucleotides or amino acid sequences can be calculated using the AlignX alignment program of the Vector NTI suite (Invitrogen, Carlsbad, Calif.). The AlignX alignment program is a global sequence alignment program for polynucleotides or proteins. Percent identity between two polynucleotides or amino acid sequences can also be calculated using the MegAlign™ (DNASTAR, Madison, Wis) software or Lasergene™ Genomics Suite Software (DNASTAR, Madison, Wis.).

### Cannabis Breeding

Cannabis is an important and valuable crop. Thus, a continuing goal of Cannabis plant breeders is to develop stable, high yielding Cannabis cultivars that are agronomically sound. To accomplish this goal, the Cannabis breeder preferably selects and develops Cannabis plants with traits that result in superior cultivars. The plants described herein can be used to produce new plant varieties. The plants can be used to develop new, unique, and superior varieties or hybrids with desired phenotypes.

The development of commercial Cannabis cultivars requires the development of Cannabis varieties, the crossing of these varieties, and the evaluation of the crosses. Pedigree breeding and recurrent selection breeding methods may be used to develop cultivars from breeding populations. Breeding programs may combine desirable traits from two or more varieties or various broad-based sources into breeding pools from which cultivars are developed by selfing and selection of desired phenotypes. The new cultivars may be crossed with other varieties and the hybrids from these crosses are evaluated to determine which have commercial potential.

Details of existing Cannabis plants varieties and breeding methods are described in Potter et al. (2011, World Wide Weed: Global Trends in Cannabis Cultivation and Its Control), Holland (2010, The Pot Book: A Complete Guide to Cannabis, Inner Traditions/Bear & Co, ISBN1594778981, 9781594778988), Green I (2009, The Cannabis Grow Bible: The Definitive Guide to Growing Marijuana for Recreational and Medical Use, Green Candy Press, 2009, ISBN 1931160589, 9781931160582), Green II (2005, The Cannabis Breeder's Bible: The Definitive Guide to Marijuana Genetics, Cannabis Botany and Creating Strains for the Seed Market, Green Candy Press, 1931160279, 9781931160278), Starks (1990, Marijuana Chemistry: Genetics, Processing & Potency, ISBN 0914171399, 9780914171393), Clarke (1981, Marijuana Botany, an Advanced Study: The Propagation and Breeding of Distinctive Cannabis, Ronin Publishing, ISBN 091417178X, 9780914171782), Short (2004, Cultivating Exceptional Cannabis: An Expert Breeder Shares His Secrets, ISBN 1936807122, 9781936807123), Cervantes (2004, Marijuana Horticulture: The Indoor/Outdoor Medical Grower's Bible, Van Patten Publishing, ISBN 187882323X, 9781878823236), Franck et al. (1990, Marijuana Grower's Guide, Red Eye Press, ISBN 0929349016, 9780929349015), Grotenhermen and Russo (2002, Cannabis and Cannabinoids: Pharmacology, Toxicology, and Therapeutic Potential, Psychology Press, ISBN 0789015080, 9780789015082), Rosenthal (2007, The Big Book of Buds: More Marijuana Varieties from the World's Great Seed Breeders, ISBN 1936807068, 9781936807062), Clarke, RC (Cannabis: Evolution and Ethnobotany 2013 (In press)), King, J (Cannabible Vols 1-3, 2001-2006), and four volumes of Rosenthal's Big Book of Buds series (2001, 2004, 2007, and 2011), each of which is herein incorporated by reference in its entirety for all purposes.

Pedigree selection, where both single plant selection and mass selection practices are employed, may be used for the generating varieties as described herein. Pedigree selection, also known as the “Vilmorin system of selection,” is described in Fehr, Walter; Principles of Cultivar Development, Volume I, Macmillan Publishing Co., which is hereby incorporated by reference. Pedigree breeding is used commonly for the improvement of self-pollinating crops or inbred lines of cross-pollinating crops. Two parents which possess favorable, complementary traits are crossed to produce an F1. An F2 population is produced by selfing one or several F1's or by intercrossing two F1's (sib mating). Selection of the best individuals usually begins in the F2 population; then, beginning in the F3, the best individuals in the best families are usually selected. Replicated testing of families, or hybrid combinations involving individuals of these families, often follows in the F4 generation to improve the effectiveness of selection for traits with low heritability. At an advanced stage of inbreeding (e.g., F6 and F7), the best lines or mixtures of phenotypically similar lines are tested for potential release as new cultivars.

Choice of breeding or selection methods depends on the mode of plant reproduction, the heritability of the trait(s) being improved, and the type of cultivar used commercially (e.g., F1 hybrid cultivar, pureline cultivar, etc.). For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, and recurrent selection.

Mass and recurrent selections can be used to improve populations of either self- or cross-pollinating crops. A genetically variable population of heterozygous individuals may be identified or created by intercrossing several different parents. The best plants may be selected based on individual superiority, outstanding progeny, or excellent combining ability. Preferably, the selected plants are intercrossed to produce a new population in which further cycles of selection are continued.

Backcross breeding has been used to transfer genes for a simply inherited, highly heritable trait into a desirable homozygous cultivar or line that is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent may be selected and repeatedly crossed (backcrossed) to the recurrent parent. The resulting plant is expected to have the attributes of the recurrent parent (e.g., cultivar) and the desirable trait transferred from the donor parent.

A single-seed descent procedure refers to planting a segregating population, harvesting a sample of one seed per plant, and using the one-seed sample to plant the next generation. When the population has advanced from the F2 to the desired level of inbreeding, the plants from which lines are derived will each trace to different F₂ individuals. The number of plants in a population declines each generation due to failure of some seeds to germinate or some plants to produce at least one seed. As a result, not all of the F2 plants originally sampled in the population will be represented by a progeny when generation advance is completed.

Mutation breeding is another method of introducing new traits into Cannabis varieties. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation (such as X-rays, Gamma rays, neutrons, Beta radiation, or ultraviolet radiation), chemical mutagens (such as base analogs like 5-bromo-uracil), antibiotics, alkylating agents (such as sulfur mustards, nitrogen mustards, epoxides, ethyleneamines, sulfates, sulfonates, sulfones, or lactones), azide, hydroxylamine, nitrous acid or acridines. Once a desired trait is observed through mutagenesis the trait may then be incorporated into existing germplasm by traditional breeding techniques. Details of mutation breeding can be found in Principles of Cultivar Development by Fehr, Macmillan Publishing Company, 1993.

The complexity of inheritance also influences the choice of the breeding method. Backcross breeding may be used to transfer one or a few favorable genes for a highly heritable trait into a desirable cultivar. This approach has been used extensively for breeding disease-resistant cultivars. Various recurrent selection techniques are used to improve quantitatively inherited traits controlled by numerous genes. The use of recurrent selection in self-pollinating crops depends on the ease of pollination, the frequency of successful hybrids from each pollination, and the number of hybrid offspring from each successful cross.

Additional breeding methods are available, e.g., methods discussed in Chahal and Gosal (Principles and procedures of plant breeding: biotechnological and conventional approaches, CRC Press, 2002, ISBN 084931321X, 9780849313219), Taji et al. (In vitro plant breeding, Routledge, 2002, ISBN 156022908X, 9781560229087), Richards (Plant breeding systems, Taylor & Francis US, 1997, ISBN 0412574500, 9780412574504), Hayes (Methods of Plant Breeding, Publisher: READ BOOKS, 2007, ISBN1406737062, 9781406737066), each of which is incorporated by reference in its entirety for all purposes. Cannabis genome has been sequenced (Bakel et al., The draft genome and transcriptome of Cannabis sativa, Genome Biology, 12(10):R102, 2011). Molecular markers for Cannabis plants are described in Datwyler et al. (Genetic variation in hemp and marijuana (Cannabis sativa L.) according to amplified fragment length polymorphisms, J Forensic Sci. 2006 March; 51(2): 371-5), Pinarkara et al., (RAPD analysis of seized marijuana (Cannabis sativa L.) in Turkey, Electronic Journal of Biotechnology, 12 (1), 2009), Hakki et al., (Inter simple sequence repeats separate efficiently hemp from marijuana (Cannabis sativa L.), Electronic Journal of Biotechnology, 10(4), 2007), Datwyler et al., (Genetic Variation in Hemp and Marijuana (Cannabis sativa L.) According to Amplified Fragment Length Polymorphisms, J Forensic Sci, March 2006, 51(2):371-375), Gilmore et al. (Isolation of microsatellite markers in Cannabis sativa L. (marijuana), Molecular Ecology Notes, 3(1):105-107, March 2003), Pacifico et al., (Genetics and marker-assisted selection of chemotype in Cannabis sativa L.), Molecular Breeding (2006) 17:257-268), and Mendoza et al., (Genetic individualization of Cannabis sativa by a short tandem repeat multiplex system, Anal Bioanal Chem (2009) 393:719-726), each of which is herein incorporated by reference in its entirety for all purposes.

The production of double haploids can also be used for the development of homozygous varieties in a breeding program. Double haploids are produced by the doubling of a set of chromosomes from a heterozygous plant to produce a completely homozygous individual. For example, see Wan et al., Theor. Appl. Genet., 77:889-892, 1989.

### Marker Assisted Selection Breeding

Some implementations of the methods disclosed herein include marker assisted selection (MAS). MAS is a powerful shortcut to selecting for desired phenotypes and for introgressing desired traits into cultivars (e.g., introgressing desired traits into elite lines). MAS is easily adapted to high throughput molecular analysis methods that can quickly screen large numbers of plant or germplasm genetic material for the markers of interest and is much more cost effective than raising and observing plants for visible traits. Thus, MAS can be used in the methods disclosed herein to produce plants with desired traits (e.g., resistance to hermaphroditism).

Introgression refers to the transmission of a desired allele of a genetic locus from one genetic background to another, which is significantly assisted through MAS. For example, introgression of a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be, e.g., a selected allele of a marker, a QTL, a transgene, or the like.

The introgression of one or more desired loci from a donor line into another is achieved via repeated backcrossing to a recurrent parent accompanied by selection to retain one or more loci from the donor parent. Markers associated with resistance to hermaphroditism may be assayed in progeny and those progeny with one or more desired markers are selected for advancement. In another aspect, one or more markers can be assayed in the progeny to select for plants with the genotype of the agronomically elite parent. Typically, trait introgressed resistance to hermaphroditism will require more than one generation, wherein progeny are crossed to the recurrent (agronomically elite) parent or selfed. Selections are made based on the presence of one or more hermaphroditism resistance markers and can also be made based on the recurrent parent genotype, wherein screening is performed on a genetic marker and/or phenotype basis. Markers disclosed herein (e.g., Tables 2, 5, and 7) can be used in conjunction with other markers, ideally at least one on each chromosome of the Cannabis genome, to track the hermaphroditism resistance phenotypes.

Genetic markers are used to identify plants that contain a desired genotype at one or more loci, and that are expected to transfer the desired genotype, along with a desired phenotype to their progeny. Genetic markers can be used to identify plants containing a desired genotype at one locus, or at several unlinked or linked loci (e.g., a haplotype), and that would be expected to transfer the desired genotype, along with a desired phenotype to their progeny. The present disclosure provides the means to identify plants that exhibit resistance to hermaphroditism by identifying plants having hermaphroditism resistance-specific markers.

In general, MAS uses polymorphic markers that have been identified as having a significant likelihood of co-segregation with a desired trait. Such markers are presumed to map near a gene or genes that give the plant its desired phenotype, and are considered indicators for the desired trait, and are termed QTL markers. Plants are tested for the presence or absence of a desired allele in the QTL marker.

Identification of plants or germplasm that include a marker locus or marker loci linked to a desired trait or traits provides a basis for performing MAS. Plants that comprise favorable markers or favorable alleles are selected for, while plants that comprise markers or alleles that are negatively correlated with the desired trait can be selected against. Desired markers and/or alleles can be introgressed into plants having a desired (e.g., elite or exotic) genetic background to produce an introgressed plant or germplasm having the desired trait. In some aspects, it is contemplated that a plurality of markers for desired traits are sequentially or simultaneously selected and/or introgressed. The combinations of markers that are selected for in a single plant are not limited, and can include any combination of markers disclosed herein or any marker linked to the markers disclosed herein, or any markers located within the QTL intervals defined herein.

A first Cannabis plant or germplasm exhibiting a desired trait (the donor) can be crossed with a second Cannabis plant or germplasm (the recipient, e.g., an elite or exotic Cannabis, depending on characteristics that are desired in the progeny) to create an introgressed Cannabis plant or germplasm as part of a breeding program. In some aspects, the recipient plant can also contain one or more loci associated with one or more desired traits, which can be qualitative or quantitative trait loci. In another aspect, the recipient plant can contain a transgene.

MAS, as described herein, using additional markers flanking either side of the DNA locus provide further efficiency because an unlikely double recombination event would be needed to simultaneously break linkage between the locus and both markers. Moreover, using markers tightly flanking a locus, a practitioner can reduce linkage drag by more accurately selecting individuals that have less of the potentially deleterious donor parent DNA. Any marker linked to or among the chromosome intervals described herein can be used.

Similarly, by identifying plants lacking a desired marker locus, plants having unfavorable resistance to hermaphroditism can be identified and eliminated from subsequent crosses. These marker loci can be introgressed into any desired genomic background, germplasm, plant, line, variety, etc., as part of an overall MAS breeding program designed to enhance resistance to hermaphroditism. Also provided are chromosome QTL intervals that can be used in MAS to select plants that demonstrate different hermaphroditism resistance traits. The QTL intervals can also be used to counter-select plants that have less favorable resistance to hermaphroditism.

Thus, the present disclosure permits one to detect the presence or absence of hermaphroditism resistance genotypes in the genomes of Cannabis plants as part of a MAS program, as described herein. In some examples, a breeder ascertains the genotype at one or more markers for a parent having favorable resistance to hermaphroditism, which contains a favorable hermaphroditism resistance allele, and the genotype at one or more markers for a parent with unfavorable resistance to hermaphroditism, which lacks the favorable hermaphroditism resistance allele. A breeder can then reliably track the inheritance of the hermaphroditism resistance alleles through subsequent populations derived from crosses between the two parents by genotyping offspring with the markers used on the parents and comparing the genotypes at those markers with those of the parents. Depending on how tightly linked the marker alleles are with the trait, progeny that share genotypes with the parent having hermaphroditism resistance alleles can be reliably predicted to express the desirable phenotype and progeny that share genotypes with the parent having unfavorable hermaphroditism resistance alleles can be reliably predicted to express the undesirable phenotype. Thus, the laborious, inefficient, and potentially inaccurate process of manually phenotyping the progeny for hermaphroditism resistance traits is avoided.

Closely linked markers flanking the locus of interest that have alleles in linkage disequilibrium with hermaphroditism resistance alleles at that locus may be effectively used to select for progeny plants with desirable resistance to hermaphroditism traits. Thus, the markers described herein, such as those listed in Tables 3 through 5, as well as other markers genetically linked to the same chromosome interval, may be used to select for Cannabis plants with different resistance to hermaphroditism traits. Often, a set of these markers will be used, (e.g., 2 or more, 3 or more, 4 or more, 5 or more) in the flanking regions of the locus. Optionally, as described above, a marker flanking or within the actual locus may also be used. The parents and their progeny may be screened for these sets of markers, and the markers that are polymorphic between the two parents used for selection. In an introgression program, this allows for selection of the gene or locus genotype at the more proximal polymorphic markers and selection for the recurrent parent genotype at the more distal polymorphic markers.

In some implementations of the disclosed methods, MAS is used to select one or more cannabis plants comprising resistance to hermaphroditism, for example, in a method comprising: i) obtaining nucleic acids from a sample plant or its germplasm; (ii) detecting one or more markers that indicate hermaphroditism resistance, and (iii) indicating hermaphroditism resistance. In some examples, the one or more markers comprises a polymorphism relative to a reference genome at nucleotide position: (a) 5,705,332 on chromosome X; (b) 5,732,323 on chromosome X; (c) 5,747,057 on chromosome X; (d) 5,877,981 on chromosome X: (e) 5,920,712 on chromosome X, (1) 6,053,325 on chromosome X, (g) 6,181,263 on chromosome X, (h) 6,186,518 on chromosome X, (i) 6,192,534 on chromosome X, (1) 6,261,819 on chromosome X, (k) 6,285,113 on chromosome X, (1) 6,695,193 on chromosome X, (m) 6,961,002 on chromosome X, (n) 17,971,672 on chromosome X, (o) 14,810,444 on chromosome 3, (p) 57,051,092 on chromosome 3, (g) 74,435,555 on chromosome 3, (r) 5,233,698 on chromosome 4, or(s) 12,961,444 on chromosome 4, wherein the reference genome is the Abacus Cannabis reference genome Csat_AbacusV2; NCBI assembly accession GCA_025232715.1. In a non-limiting example, the one or more markers comprise a polymorphism at one or more of nucleotide position 5,732,323 on chromosome X, nucleotide position 17,971,672 on chromosome X, and nucleotide position 14,810,444 on chromosome 3. In a further example, the one or more markers comprise a polymorphism at nucleotide position 5,732,323 on chromosome X, nucleotide position 17,971,672 on chromosome X, and nucleotide position 14,810,444 on chromosome 3. In another example, the one or more markers consist of, or essentially consist of, a polymorphism at nucleotide position 5,732,323 on chromosome X, nucleotide position 17,971,672 on chromosome X, and nucleotide position 14,810,444 on chromosome 3.

In some examples, the nucleotide position comprises: (a) on chromosome X: (1) an A/A or G/A genotype at position 5,705,332; (2) an A/A or G/A genotype at position 5,732,323; (3) a G/G or A/G genotype at position 5,747,057; (4) a T/T C/T genotype at position 5,877,981; (5) a C/C or A/C genotype at position 5,920,712; (6) a T/T or C/T genotype at position 6,053,325; (7) a T/T or C/T genotype at position 6,181,263; (8) an A/A or G/A genotype at position 6,186,518; (9) a C/C or A/C genotype at position 6,192,534; (10) an A/A or G/A genotype at position 6,261,819; (11) an A/A or G/A genotype at position 6,285,113; (12) an A/A or C/A genotype at position 6,695,193; (13) a T/T or C/T genotype at position 6,961,002; or (14) a T/T or C/T genotype at position 17,971,672; (b) on chromosome 3: (1) a T/T or C/T genotype at position 14,810,444; (2) a G/G or G/A genotype at position 57,051,092; or (3) a C/C genotype at position 74,435,555; (c) on chromosome 4: (1) a G/G or G/A genotype at position 5,233,698; or (2) a G/G genotype at position 12,961,444; wherein the reference genome is the Abacus Cannabis reference genome Csat_AbacusV2; NCBI assembly accession GCA_025232715.1. In a non-limiting example, the nucleotide position comprises an A/A or G/A genotype at position 5,732,323 on chromosome X, a T/T or C/T genotype at position 17,971,672, or a T/T or C/T genotype at position 14,810,444. In a further example, the nucleotide position comprises an A/A or G/A genotype at position 5,732,323 on chromosome X, a T/T or C/T genotype at position 17,971,672 on chromosome X, and a T/T or C/T genotype at position 14,810,444 on chromosome 3. In another example, the nucleotide position consists of, or essentially consists of, an A/A or G/A genotype at position 5,732,323 on chromosome X, a T/T or C/T genotype at position 17,971,672 on chromosome X, and a T/T or C/T genotype at position 14,810,444 on chromosome 3.

In some examples, the one or more markers comprises a polymorphism at position 26 of any one or more of SEQ ID NO:1; SEQ ID NO:2; SEQ ID NO:3; SEQ ID NO:4; SEQ ID NO:5; SEQ ID NO:6; SEQ ID NO: 7; SEQ ID NO:8; SEQ ID NO:9; SEQ ID NO:10; SEQ ID NO: 11; SEQ ID NO:12; SEQ ID NO:13; SEQ ID NO:14; SEQ ID NO: 15; SEQ ID NO:16; SEQ ID NO:17; SEQ ID NO:18; or SEQ ID NO:19. In some examples, the nucleotide position comprises: (1) an A/A or G/A genotype at position 26 of SEQ ID NO: 1; (2) an A/A or G/A genotype at position 26 of SEQ ID NO:2; (3) a G/G or A/G genotype at position 26 of SEQ ID NO:3; (4) a T/T or C/T genotype at position 26 of SEQ ID NO:4; (5) a C/C or A/C genotype at position 26 of SEQ ID NO:5; (6) a T/T or C/T genotype at position 26 of SEQ ID NO:6; (7) a T/T or C/T genotype at position 26 of SEQ ID NO:7; (8) an A/A or G/A genotype at position 26 of SEQ ID NO:8; (9) a C/C or A/C genotype at position 26 of SEQ ID NO:9; (10) an A/A or G/A genotype at position 26 of SEQ ID NO: 10; (11) an A/A or G/A genotype at position 26 of SEQ ID NO:11; (12) an A/A or C/A genotype at position 26 of SEQ ID NO: 12; (13) a T/T or C/T genotype at position 26 of SEQ ID NO: 13; (14) a T/T or C/T genotype at position 26 of SEQ ID NO:14; (15) a T/T or C/T genotype at position 26 of SEQ ID NO:15; (16) a G/G or G/A genotype at position 26 of SEQ ID NO:16; (17) a C/C genotype at position 26 of SEQ ID NO: 17; (18) a G/G or G/A genotype at position 26 of SEQ ID NO:18; or (19) a G/G genotype at position 26 of SEQ ID NO: 19; wherein the reference genome is the Abacus Cannabis reference genome Csat_AbacusV2; NCBI assembly accession GCA_025232715.1. In an example, the nucleotide position comprises an A/A or G/A genotype at position 26 of SEQ ID NO:2; a T/T or C/T genotype at position 26 of SEQ ID NO:14; or a T/T or C/T genotype at position 26 of SEQ ID NO:15. In another example, the nucleotide position comprises at least two nucleotide positions selected from: an A/A or G/A genotype at position 26 of SEQ ID NO:2; a T/T or C/T genotype at position 26 of SEQ ID NO:14; and a T/T or C/T genotype at position 26 of SEQ ID NO:15. In a further example, the nucleotide position comprises at least three nucleotide positions: an A/A or G/A genotype at position 26 of SEQ ID NO:2; a T/T or C/T genotype at position 26 of SEQ ID NO:14; and a T/T or C/T genotype at position 26 of SEQ ID NO:15.

In further examples, the one or more markers comprises a polymorphism at position 51 of any one or more of SEQ ID NO:62; SEQ ID NO:63; SEQ ID NO:64; SEQ ID NO:65; SEQ ID NO:66; SEQ ID NO: 67; SEQ ID NO:68; SEQ ID NO:69; SEQ ID NO:70; SEQ ID NO:71; SEQ ID NO:72; SEQ ID NO:73; SEQ ID NO:74; SEQ ID NO:75; SEQ ID NO:76; SEQ ID NO:77; SEQ ID NO:78; SEQ ID NO:79; or SEQ ID NO: 80. In some examples, the nucleotide position comprises: (1) an A/A or G/A genotype at position 51 of SEQ ID NO:62; (2) an A/A or G/A genotype at position 51 of SEQ ID NO:63; (3) a G/G or A/G genotype at position 51 of SEQ ID NO:64; (4) a T/T or C/T genotype at position 51 of SEQ ID NO:65; (5) a C/C or A/C genotype at position 51 of SEQ ID NO:66; (6) a T/T or C/T genotype at position 51 of SEQ ID NO:67; (7) a T/T or C/T genotype at position 51 of SEQ ID NO:68; (8) an A/A or G/A genotype at position 51 of SEQ ID NO:69; (9) a C/C or A/C genotype at position 51 of SEQ ID NO:70; (10) an A/A or G/A genotype at position 51 of SEQ ID NO:71; (11) an A/A or G/A genotype at position 51 of SEQ ID NO:72; (12) an A/A or C/A genotype at position 51 of SEQ ID NO:73; (13) a T/T or C/T genotype at position 51 of SEQ ID NO: 74; (14) a T/T or C/T genotype at position 51 of SEQ ID NO:75; (15) a T/T or C/T genotype at position 51 of SEQ ID NO:76; (16) a G/G or G/A genotype at position 51 of SEQ ID NO:77; (17) a C/C genotype at position 51 of SEQ ID NO:78; (18) a G/G or G/A genotype at position 51 of SEQ ID NO:79; or (19) a G/G genotype at position 51 of SEQ ID NO:80; wherein the reference genome is the Abacus Cannabis reference genome Csat_AbacusV2; NCBI assembly accession GCA_025232715.1. In an example, the nucleotide position comprises an A/A or G/A genotype at position 51 of SEQ ID NO:63; a T/T or C/T genotype at position 51 of SEQ ID NO:75; or a T/T or C/T genotype at position 51 of SEQ ID NO:76. In another example, the nucleotide position comprises at least two nucleotide positions selected from: an A/A or G/A genotype at position 51 of SEQ ID NO:63; a T/T or C/T genotype at position 51 of SEQ ID NO:75; and a T/T or C/T genotype at position 51 of SEQ ID NO:76. In a further example, the nucleotide position comprises at least three nucleotide positions: an A/A or G/A genotype at position 51 of SEQ ID NO:63; a T/T or C/T genotype at position 51 of SEQ ID NO:75; and a T/T or C/T genotype at position 51 of SEQ ID NO: 76.

A number of SNPs together within a sequence, or across linked sequences, can be used to describe a haplotype for any particular genotype (Ching et al. (2002), BMC Genet. 3:19 pp Gupta et al. 2001, Rafalski (2002b), Plant Science 162:329-333). Haplotypes may in some circumstances be more informative than single SNPs and can be more descriptive of any particular genotype. Exemplary haplotypes are described herein (e.g., Table 2, 5, and 7), and can be used for marker assisted selection. In one example, the one or more markers comprise a polymorphism relative to a reference genome within any one or more haplotypes, wherein the haplotypes comprise the region: (a) on chromosome X: (1) between positions 5,696,400 and 5,714,336; (2) between positions 5,725,231 and 5,737,968; (3) between positions 5,737,968 and 5,750,301; (4) between positions 5,860,363 and 5,894,065; (5) between positions 5,894,065 and 5,929,330; (6) between positions 6,032,408 and 6,059,371; (7) between positions 6,133,760 and 6,189,246; (8) between positions 6,189,246 and 6,197,726; (9) between positions 6,258,772 and 6,290,824; (10) between positions 6,688,670 and 6,715,018; (11) between positions 6,932,849 and 6,970,023; (12) between positions 17,900,648 and 17,980,443; (b) on chromosome 3: (1) between positions 14,797,288 and 14,824,861; (2) between positions 56,616,473 and 57,090,960; or (3) between positions 74,422,393 and 74,437,841; (c) on chromosome 4: (1) between positions 5,216,694 and 5,243,017; or (2) between positions 12,923,613 and 13,009,666; wherein the reference genome is the Abacus Cannabis reference genome Csat_AbacusV2; NCBI assembly accession GCA_025232715.1. In some examples, the haplotype includes the region between positions 5,725,231 and 5,737,968 on chromosome X, the region between positions 17,900,648 and 17,980,443 on chromosome X, or the region between positions 14,797,288 and 14,824,861 on chromosome 3. In some examples, the haplotype includes at least two of the region between positions 5,725,231 and 5,737,968 on chromosome X, the region between positions 17,900,648 and 17,980,443 on chromosome X, and the region between positions 14,797,288 and 14,824,861 on chromosome 3. In some examples, the haplotype includes the region between positions 5,725,231 and 5,737,968 on chromosome X, the region between positions 17,900,648 and 17,980,443 on chromosome X, and the region between positions 14,797,288 and 14,824,861 on chromosome 3.

The choice of markers actually used is not limited and can be any marker that is genetically linked to the intervals as described herein, which includes markers mapping within the intervals. In certain implementations of the present disclosure, markers closely genetically linked to, or within approximately 0.5 cM of, the markers provided herein and chromosome intervals whose borders fall between or include such markers, including markers within approximately 0.4 cM, 0.3 cM, 0.2 cM, and about 0.1 cM of the markers provided herein, are used.

The markers and haplotypes described above can be used for marker assisted selection to produce additional progeny plants comprising the indicated resistance to hermaphroditism. In some examples, backcrossing is used in conjunction with marker-assisted selection.

### Genetic Engineering

Disclosed herein are modified cannabis plants comprising a non-naturally occurring genetic modification in: (a) switch/sucrose nonfermenting 3C (SWI3C), (b) catalase 2 (CAT2), or (c) 1-aminocyclopropane-1-carboxylate synthase 12 (ACS12); or comprising a heterologous beneficial allele of SWI3C, CAT2, or ACS12. In some examples, the modified cannabis plants include a non-naturally occurring combination of beneficial alleles of SWI3C, CAT2, and ACS12. Exemplary beneficial alleles of SWI3C, CAT2, and ACS12 (and beneficial combinations thereof) are disclosed herein.

The present disclosure includes genetic engineering (e.g., gene or genome editing) of plants to develop plants having resistance to hermaphroditism. In particular, disclosed herein are methods for selecting one or more cannabis plants comprising resistance to hermaphroditism, the method comprising: (1) replacing a nucleic acid sequence of a parent plant with a nucleic acid sequence conferring resistance to hermaphroditism, (ii) crossing or selfing the parent plant, thereby producing a plurality of progeny seed, and (iii), selecting one or more progeny plants grown from the progeny seed that comprise the nucleic acid sequence conferring resistance to hermaphroditism, thereby selecting plants having resistance to hermaphroditism.

In some aspects, the method comprises introducing a genetic modification in SWI3C, CAT2, or ACS12. In some examples, the genetic modification is associated with resistance to hermaphroditism. In some examples, the genetic modification increases resistance to hermaphroditism relative to the plant in an unmodified state. In some examples, the genetic modification is a nucleic acid substitution, insertion, or deletion. In some examples, the genetic modification is introduced by mutagenesis or a gene editing technique (e.g., RNAi, CRISPR/Cas9, or TALEN based systems). In some examples, a genetic modification is introduced in two or more of SWI3C, CAT2, and ACS12. In a non-limiting example, a genetic modification is introduced in SWI3C, CAT2, and ACS12. Non-limiting, exemplary genetic modifications of SWI3C include a substitution at a position corresponding to 728 bp in the coding sequence from the start codon of Abacus SWI3C (position 5,726,701 bp on chromosome X of the Abacus reference genome); and a substitution at a position corresponding to 1193 bp in the coding sequence from the start codon of Abacus SWI3C (position 5,727,731 bp on chromosome X of the Abacus reference genome). Non-limiting, exemplary genetic modifications of CAT2 include a substitution at a position corresponding to 338 bp from the start of the 3′UTR of Abacus CAT2 (position 17,972,352 bp on chromosome X of the Abacus reference genome); and an insertion starting at a position corresponding to 394 bp from the start of the 3′UTR of Abacus CAT2. Non-limiting, exemplary genetic modifications of ACS12 include a deletion at a position corresponding to 1500 bp from the start codon of Abacus ACS12 (position 17,972,408 bp on chromosome X of the Abacus reference genome); an insertion at a position corresponding to 25 bp from the start of the 3′UTR of Abacus ACS12 (position 14,804,986 bp on chromosome 3 of the Abacus reference genome); and a substitution at a position corresponding to 31 bp from the start of the 3′UTR of Abacus ACS12 (position 14,804,992 bp on chromosome 3 of the Abacus reference genome). The Abacus Cannabis reference genome refers to Csat_AbacusV2; NCBI assembly accession GCA_025232715.1. In a non-limiting example, the substitution at a position corresponding to 728 bp of Abacus SWT3C CDS is a G. In a non-limiting example, the substitution at a position corresponding to 1193 bp of Abacus SWI3C CDS is a C. In a non-limiting example, the substitution at a position corresponding to 338 bp from the start of the 3′UTR of Abacus CAT2 is a C. In a non-limiting example, the insertion starting at a position corresponding to 394 bp from the start of the 3′UTR of Abacus CAT2 is an insertion of CTGATAT. In a non-limiting example, the deletion corresponding to a position 1500 bp from the start codon of Abacus ACS12 is a deletion of TACCGAAAC or TACCGAAAG. In a non-limiting example, the insertion at a position corresponding to 25 bp from the start of the 3′UTR of Abacus ACS12 is TTTT. In a non-limiting example the substitution corresponding to a position 31 bp from the start of the 3′UTR of Abacus ACS12 is a C.

In some examples the modification is homozygous or heterozygous in the modified plant. In some examples, the modification is homozygous in the modified plant.

In some implementations, the genetic modification is introduced by mutagenesis or gene editing. Gene editing (genome editing) is well known and described. For example, the ability to engineer a trait relies on the action of the genome editing proteins and various endogenous DNA repair pathways. These pathways may be normally present in a cell or may be induced by the action of the genome editing protein. Using genetic and chemical tools to over-express or suppress one or more genes or elements of these pathways can improve the efficiency and/or outcome of gene editing. For example, it can be useful to over-express certain homologous recombination pathway genes or suppress non-homologous pathway genes, depending upon the desired modification.

For example, gene function can be modified using antisense modulation using at least one antisense compound, including antisense DNA, antisense RNA, a ribozyme, DNAzyme, a locked nucleic acid (LNA) and an aptamer. In some examples, the molecules are chemically modified. In some examples, the antisense molecule is antisense DNA or an antisense DNA analog.

RNA interference (RNAi) is another method known in the art to reduce gene function in plants, which is mediated by RNA-induced silencing complex (RISC), a sequence-specific, multicomponent nuclease that destroys messenger RNAs homologous to the silencing trigger. RISC is known to contain short RNAs (approximately 22 nucleotides) derived from the double-stranded RNA trigger. The short-nucleotide RNA sequences are homologous to the target gene that is being suppressed. Thus, the short-nucleotide sequences appear to serve as guide sequences to instruct a multicomponent nuclease, RISC, to destroy the specific mRNAs. The dsRNA used to initiate RNAi, may be isolated from native source or produced by known means, e.g., transcribed from DNA. Plasmids and vectors for generating RNAi molecules against target sequence are now readily available from commercial sources.

DNAzyme molecules, enzymatic oligonucleotides, and mutagenesis are other commonly known methods for reducing gene function. Any available mutagenesis procedure can be used, including but not limited to, site-directed point mutagenesis, random point mutagenesis, in vitro or in vivo homologous recombination (DNA shuffling), uracil-containing templates, oligonucleotide-directed mutagenesis, phosphorothioate-modified DNA mutagenesis, mutagenesis using gapped duplex DNA, point mismatch repair, repair-deficient host strains, restriction-selection and restriction-purification, deletion mutagenesis, total gene synthesis, double-strand break repair, zinc-finger nucleases (ZFN), or transcription activator-like effector nucleases (TALEN).

Clustered regularly interspaced short palindromic repeats (CRISPR)/CRISPR associated protein (Cas) system comprises genome engineering tools based on the bacterial CRISPR/Cas prokaryotic adaptive immune system. This RNA-based technology is very specific and allows targeted cleavage of genomic DNA guided by a customizable small noncoding RNA, resulting in gene modifications by both non-homologous end joining (NHEJ) and homology-directed repair (HDR) mechanisms (Belbaj K. et al., 2013. Plant Methods 2013, 9:39). A non-limiting example of a CRISPR/Cas system includes a CRISPR/Cas9 system. In some examples, the target cell expresses a Cas nuclease (e.g., Cas9), and a CRISPR RNA is expressed or transformed into the cell. In some examples, a nucleoprotein complex comprising a Cas nuclease (e.g., Cas9) and a CRISPR RNA is transformed into the target cell. CRISPR-based gene editing systems need not be limited to Cas9 systems, as other suitable/analogous editing enzymes have been described, e.g., MAD7.

In some implementations, the method of producing a genetically engineered cannabis plant comprises introducing a heterologous gene, for example, a beneficial allele (hermaphroditism resistant) of SWI3C, CAT2, and/or ACS12. In some examples, the beneficial allele of SWI3C is a SWI3C allele from 21TP1B-1-1 or 21TP1B-21-1. In some examples, the beneficial allele of SWI3C includes an arginine at a position corresponding to amino acid 242 of Abacus SWI3C and/or a threonine at a position corresponding to amino acid 398 of Abacus SWI3C (positions refer to Abacus SWI3C protein sequence, see, SEQ ID NO: 37). In some examples, the beneficial allele of SWI3C includes a substitution at position 728 bp from the start codon of the coding sequence of Abacus SWI3C (SEQ ID NO: 33) and/or a substitution at position 1193 bp from the start codon of the coding sequence of Abacus SWI3C (SEQ ID NO: 33). In some examples, the beneficial allele CAT2 allele is a CAT2 allele from 21TP1B-21-1 or 21TCV1-4-5. In some examples, the beneficial allele of CAT2 includes a substitution at a position corresponding to position 338 bp from the start of the 3′UTR of Abacus CAT2 (SEQ ID NO: 44) and/or an insertion starting at a position corresponding to 394 bp from the start of the 3′UTR of Abacus CAT2 (SEQ ID NO: 44). In some examples, the beneficial ACS12 allele is an ACS12 allele from 20TP1B-1020-1 or Abacus. In some examples, the beneficial allele of ACS12 includes a deletion at a position corresponding to 1500 bp from the start codon of the coding sequence of Abacus ACS12 (SEQ ID NO. 50), and/or an insertion starting at a position corresponding to 25 bp from the start of the 3′UTR of Abacus ACS12 (SEQ ID NO: 58) or a substitution at a position corresponding to 31 bp from the start of the 3′UTR of Abacus ACS12 (SEQ ID NO: 58). In some examples, the beneficial allele increases resistance to hermaphroditism relative to the cannabis plant in an unmodified state.

Methods for transformation of plant cells required for gene editing are well known in the art, and the selection of the most appropriate transformation technique can be determined by the practitioner. Suitable methods may include electroporation of plant protoplasts, liposome-mediated transformation, polyethylene glycol (PEG) mediated transformation, transformation using viruses, micro-injection of plant cells, micro-projectile bombardment of plant cells, and Agrobacterium tumefaciens mediated transformation. Transformation means introducing a nucleotide sequence in a plant in a manner to cause stable or transient expression of the sequence.

In planta transformation techniques (e.g., vacuum-infiltration, floral spraying or floral dip procedures) are well known and may be used to introduce expression cassettes (typically in an Agrobacterium vector) into meristematic or germline cells of a whole plant. Such methods provide a simple and reliable method of obtaining transformants at high efficiency while avoiding the use of tissue culture. (see, e.g., Bechtold et at. 1993 C. R. Acad. Sci. 316:1194-1199; Chung et at. 2000 Transgenic Res. 9:471-476; Clough et al. 1998 Plant J. 16:735-743; and Desfeux et at. 2000 Plant Physiol 123:895-904). In these techniques, seed produced by the plant comprise the expression cassettes encoding the genes of interest. The seed can be selected based on the ability to germinate under conditions that inhibit germination of the untransformed seed.

If transformation techniques require use of tissue culture, transformed cells may be regenerated into plants in accordance with techniques well known to those of skill in the art. The regenerated plants may then be grown, and crossed with the same or different plant varieties using traditional breeding techniques to produce seed, which are then selected under the appropriate conditions.

The expression cassette can be integrated into the genome of the plant cells, in which case subsequent generations will express the gene of interest. Alternatively, the expression cassette is not integrated into the genome of the plant's cell, in which case the genome editing protein is transiently expressed in the transformed cells and is not expressed in subsequent generations.

A genome editing protein itself may be introduced into the plant cell in a sufficient quantity to modify the cell, but does not persist after a contemplated period of time has passed or after one or more cell divisions. In such examples, no further steps are needed to remove or segregate away the genome editing protein and the modified cell. The genome editing protein can be prepared in vitro prior to introduction to a plant cell using well known recombinant expression systems (bacterial expression, in vitro translation, yeast cells, insect cells and the like). After expression, the protein is isolated, refolded if needed, purified and optionally treated to remove any purification tags, such as a His-tag. Once crude, partially purified, or more completely purified genome editing proteins are obtained, they may be introduced to a plant cell via electroporation, by bombardment with protein coated particles, by chemical transfection or by some other means of transport across a cell membrane.

The genome editing protein can also be expressed, for example in Agrobacterium, as a fusion protein fused to an appropriate domain of a virulence protein that is translocated into plants (e.g., VirD2, VirE2, VirE2 and VirF). The Vir protein fused with the genome editing protein travels to the plant cell's nucleus, where the genome editing protein would produce the desired double stranded break in the genome of the cell. (see, e.g., Vergunst et ar. Science 290:979-82, 2000).

### Kits for Use in Diagnostic Applications

Kits for use in diagnostic, research, and prognostic applications are also provided. Such kits may include any or all of the following: assay reagents, buffers, nucleic acids for detecting the target sequences and other hybridization probes and/or primers. The kits may include instructional materials containing directions (i.e., protocols) for the practice of the methods of the present disclosure. While the instructional materials typically comprise written or printed materials, they are not limited to such. Any medium capable of storing such instructions and communicating them to an end user is contemplated. Such media include, but are not limited to, electronic storage media (e.g., magnetic discs, tapes, cartridges, chips), optical media (e.g., CD ROM), cloud-based media, and the like. Such media may include addresses to internet sites that provide such instructional materials.

### ADDITIONAL ASPECTS OF THE DISCLOSURE

[Clause 1] A method for selecting one or more plants having resistance to hermaphroditism, the method comprising i) obtaining nucleic acids from a sample plant or its germplasm; (ii) detecting one or more markers that indicate resistance to hermaphroditism, and (iii) indicating resistance to hermaphroditism.
[Clause 2] The method of clause 1 further comprising selecting the one or more plants indicating resistance to hermaphroditism.
[Clause 3] The method of clause 1 wherein the one or more markers comprises a polymorphism relative to a reference genome at nucleotide position: (a) 5,705,332 on chromosome X; (b) 5,732,323 on chromosome X; (c) 5,747,057 on chromosome X; (d) 5,877,981 on chromosome X; (e) 5,920,712 on chromosome X, (f) 6,053,325 on chromosome X, (g) 6,181,263 on chromosome X, (h) 6,186,518 on chromosome X, (i) 6,192,534 on chromosome X, (j) 6,261,819 on chromosome X, (k) 6,285,113 on chromosome X, (l) 6,695,193 on chromosome X, (m) 6,961,002 on chromosome X, (n) 17,971,672 on chromosome X, (o) 14,810,444 on chromosome 3, (p) 57,051,092 on chromosome 3, (q) 74,435,555 on chromosome 3, (r) 5,233,698 on chromosome 4, or(s) 12,961,444 on chromosome 4, wherein the reference genome is the Abacus Cannabis reference genome (version CsaAba2).
[Clause 4] The method of clause 3 wherein the nucleotide position comprises: (a) on chromosome X: (1) an A/A genotype at position 5,705,332; (2) an A/A genotype at position 5,732,323; (3) a G/G genotype at position 5,747,057; (4) a T/T genotype at position 5,877,981; (5) a C/C genotype at position 5,920,712; (6) a T/T genotype at position 6,053,325; (7) a T/T genotype at position 6,181,263; (8) an A/A genotype at position 6,186,518; (9) a C/C genotype at position 6,192,534; (10) an A/A genotype at position 6,261,819; (11) an A/A genotype at position 6,285,113; (12) an A/A genotype at position 6,695,193; (13) a T/T genotype at position 6,961,002; or (14) a T/T genotype at position 17,971,672; (b) on chromosome 3: (1) a T/T genotype at position 14,810,444; (2) a G/G genotype at position 57,051,092; or (3) a C/C genotype at position 74,435,555; (c) on chromosome 4: (1) a G/G or G/A genotype at position 5,233,698; or (2) a G/G genotype at position 12,961,444; wherein the reference genome is the Abacus Cannabis reference genome (version CsaAba2).
[Clause 5] The method of clause 1 wherein the one or more markers comprises a polymorphism at position 26 of any one or more of SEQ ID NO:1; SEQ ID NO:2; SEQ ID NO:3; SEQ ID NO:4; SEQ ID NO: 5; SEQ ID NO:6; SEQ ID NO:7; SEQ ID NO:8; SEQ ID NO:9; SEQ ID NO:10; SEQ ID NO:11; SEQ ID NO: 12; SEQ ID NO:13; SEQ ID NO:14; SEQ ID NO:15; SEQ ID NO:16; SEQ ID NO: 17; SEQ ID NO: 18; or SEQ ID NO:19.
[Clause 6] The method of clause 5 wherein the nucleotide position comprises: (1) an A/A genotype at position 26 of SEQ ID NO:1; (2) an A/A genotype at position 26 of SEQ ID NO:2; (3) a G/G genotype at position 26 of SEQ ID NO:3; (4) a T/T genotype at position 26 of SEQ ID NO:4; (5) a C/C genotype at position 26 of SEQ ID NO:5; (6) a T/T genotype at position 26 of SEQ ID NO:6; (7) a T/T genotype at position 26 of SEQ ID NO:7; (8) an A/A genotype at position 26 of SEQ ID NO:8: (9) a C/C genotype at position 26 of SEQ ID NO:9; (10) an A/A genotype at position 26 of SEQ ID NO:10; (11) an A/A genotype at position 26 of SEQ ID NO:11; (12) an A/A genotype at position 26 of SEQ ID NO: 12; (13) a T/T genotype at position 26 of SEQ ID NO:13; (14) a T/T genotype at position 26 of SEQ ID NO:14; (15) a T/T genotype at position 26 of SEQ ID NO:15; (16) a G/G genotype at position 26 of SEQ ID NO: 16; (17) a C/C genotype at position 26 of SEQ ID NO:17; (18) a G/G or G/A genotype at position 26 of SEQ ID NO:18; or (19) a G/G genotype at position 26 of SEQ ID NO: 19; wherein the reference genome is the Abacus Cannabis reference genome (version CsaAba2).
[Clause 7] The method of clause 1 wherein the one or more markers comprises a polymorphism relative to a reference genome within any one or more haplotypes wherein the haplotypes comprise the region: (a) on chromosome X: (1) between positions 5,696,400 and 5,714,336; (2) between positions 5,725,231 and 5,737,968; (3) between positions 5,737,968 and 5,750,301; (4) between positions 5,860,363 and 5,894,065; (5) between positions 5,894,065 and 5,929,330; (6) between positions 6,032,408 and 6,059,371; (7) between positions 6,133,760 and 6,189,246; (8) between positions 6,189,246 and 6,197,726; (9) between positions 6,258,772 and 6,290,824; (10) between positions 6,688,670 and 6,715,018; (11) between positions 6,932,849 and 6,970,023; (12) between positions 17,900,648 and 17,980,443; (b) on chromosome 3: (1) between positions 14,797,288 and 14,824,861; (2) between positions 56,616,473 and 57,090,960; or (3) between positions 74,422,393 and 74,437,841; (c) on chromosome 4: (1) between positions 5,216,694 and 5,243,017; or (2) between positions 12,923,613 and 13,009,666; wherein the reference genome is the Abacus Cannabis reference genome (version CsaAba2).
[Clause 8] The method of clause 1 wherein the selecting comprises marker assisted selection.
[Clause 9] The method of clause 1 wherein the detecting comprises an oligonucleotide probe.
[Clause 10] The method of clause 1 further comprising crossing the one or more plants comprising the indicated resistance to hermaphroditism to produce one or more F1 or additional progeny plants, wherein at least one of the F1 or additional progeny plants comprises the indicated resistance to hermaphroditism.
[Clause 11] The method of clause 10 wherein the crossing comprises selfing, sibling crossing, or backcrossing.
[Clause 12] The method of clause 10 wherein the at least one additional progeny plant comprising the indicated resistance to hermaphroditism comprises an F2-F7 progeny plant.
[Clause 13] The method of clause 11 wherein the selfing, sibling crossing, or backcrossing comprises marker-assisted selection.
[Clause 14] The method of clause 11 wherein the selfing, sibling crossing, or backcrossing comprises marker-assisted selection for at least two generations.
[Clause 15] The method of clause 1 wherein the plant comprises a Cannabis plant.
[Clause 16] A method for selecting one or more plants comprising resistance to hermaphroditism, the method comprising replacing a nucleic acid sequence of a parent plant with a nucleic acid sequence conferring resistance to hermaphroditism.

### EXAMPLES

Aspects of the present teachings can be further understood in light of the following examples, which should not be construed as limiting the scope of the present teachings in any way.

### Example 1

### Discovery of Markers Associated with Hermaphroditism

### Comprehensive Mapping

Cannabis and hemp germplasm from 205 diverse seed lots (n=1317; Table 1) were genotyped using an Illumina bead array to map single nucleotide polymorphisms (SNPs) associated with hermaphroditism across multiple genetic backgrounds. Plants were grown in a greenhouse or field under standard growing conditions. Plants were regularly inspected for the formation of hermaphroditic flowers. Since hermaphroditic cannabis flowers tend to develop as a result of stress, which is not always present throughout the greenhouse or field, there is a possibility that plants phenotyped as female flowering could develop hermaphroditic flowers when grown under different conditions.

TABLE 1
Seed lots used for association mapping in a set of 205 diverse seed lots. First column: identifier
of seed lots grown in the greenhouse under standard growing conditions, *= plants grown
in the field; second column: number of hermaphroditic accessions per seed lot used for mapping;
third column: number of female flowering accessions per seed lot used for mapping; fourth column:
total number of accessions per seed lot used for mapping; fifth column: total number of accessions
evaluated for incidence of hermaphroditism; sixth column: percentage of hermaphroditic accessions
in total number of accessions evaluated for incidence of hermaphroditism.

| | Number of | Number of | | | Percentage |
|---|---|---|---|---|---|
| | hermaphroditic | female | Total number | Total number | hermaphroditic in |
| | accessions for | accessions for | of accessions | of accessions | total number of |
| Seed lot | mapping | mapping | for mapping | evaluated | accessions evaluated |
| 19AMTY-3* | 0 | 8 | 8 | 250 | 0% |
| 19AMTY-4* | 12 | 7 | 19 | 241 | 5% |
| 19AMTY-5* | 0 | 5 | 5 | 120 | 0% |
| 19AMTY-6* | 2 | 11 | 13 | 120 | 2% |
| 19AMTY-7* | 3 | 9 | 12 | 120 | 3% |
| 19AMTY-8* | 3 | 15 | 18 | 120 | 3% |
| 19AMTY-9* | 11 | 3 | 14 | 255 | 4% |
| 19GAR1-1 | 1 | 6 | 7 | 7 | 14% |
| 19GAR1-2 | 1 | 3 | 4 | 4 | 25% |
| 19GAR1-3 | 0 | 6 | 6 | 6 | 0% |
| 19GAR1-4 | 1 | 5 | 6 | 6 | 17% |
| 19GAR1-5 | 1 | 4 | 5 | 5 | 20% |
| 19GAR1-6 | 0 | 3 | 3 | 3 | 0% |
| 19GAR1-7 | 0 | 3 | 3 | 3 | 0% |
| 19GAR1-8 | 4 | 0 | 4 | 4 | 100% |
| 19GAR1-9 | 6 | 0 | 6 | 6 | 100% |
| 19GAR1-10 | 2 | 0 | 2 | 2 | 100% |
| 19GAR1-12 | 0 | 3 | 3 | 3 | 0% |
| 19GAR1-16 | 0 | 5 | 5 | 5 | 0% |
| 19GAR1-17 | 3 | 3 | 6 | 6 | 50% |
| 19GAR1-18 | 2 | 0 | 2 | 2 | 100% |
| 19GAR1-19 | 2 | 4 | 6 | 6 | 33% |
| 19GAR1-20 | 5 | 0 | 5 | 5 | 100% |
| 19GAR1-21 | 2 | 0 | 2 | 2 | 100% |
| 19GAR1-22 | 1 | 0 | 1 | 1 | 100% |
| 19GAR1-23 | 0 | 2 | 2 | 2 | 0% |
| 19GAR1-24 | 0 | 8 | 8 | 8 | 0% |
| 19GAR1-25 | 0 | 7 | 7 | 6 | 0% |
| 19GAR1-26 | 2 | 0 | 2 | 2 | 100% |
| 19GAR1-27 | 0 | 4 | 4 | 4 | 0% |
| 19GAR1-28 | 1 | 3 | 4 | 4 | 25% |
| 19GAR1-29 | 3 | 0 | 3 | 3 | 100% |
| 19GAR1-30 | 0 | 4 | 4 | 4 | 0% |
| 19GAR1-32 | 1 | 2 | 3 | 3 | 33% |
| 19GAR1-34 | 0 | 6 | 6 | 6 | 0% |
| 19GAR1-36 | 1 | 2 | 3 | 3 | 33% |
| 19GAR1-38 | 0 | 2 | 2 | 2 | 0% |
| 19GAR1-44 | 0 | 11 | 11 | 11 | 0% |
| 19GAR1-45 | 1 | 13 | 14 | 14 | 7% |
| 19GAR1-47 | 2 | 9 | 11 | 11 | 18% |
| 19GAR1-48 | 0 | 9 | 9 | 9 | 0% |
| 19GAR1-49 | 1 | 7 | 8 | 8 | 13% |
| 19GAR1-50 | 2 | 13 | 15 | 15 | 13% |
| 19GAR1-52 | 0 | 20 | 20 | 20 | 0% |
| 19GAR1-53 | 1 | 2 | 3 | 3 | 33% |
| 19GAR1-54 | 3 | 5 | 8 | 8 | 38% |
| 19GAR1-55 | 4 | 0 | 4 | 4 | 100% |
| 19GAR1-56 | 1 | 4 | 5 | 5 | 20% |
| 19GAR1-57 | 6 | 2 | 8 | 8 | 75% |
| 20COW-1 | 5 | 4 | 9 | 20 | 75% |
| 20COW-2 | 5 | 2 | 7 | 28 | 86% |
| 20COW-3 | 3 | 1 | 4 | 7 | 71% |
| 20COW-4 | 2 | 2 | 4 | 8 | 38% |
| 20COW-8 | 0 | 6 | 6 | 26 | 0% |
| 20COW-9 | 4 | 6 | 10 | 32 | 19% |
| 20COW-9 | 0 | 5 | 5 | 31 | 0% |
| 20COW-11 | 3 | 3 | 6 | 23 | 48% |
| 20COW-13 | 4 | 1 | 5 | 20 | 75% |
| 20COW-14 | 3 | 1 | 4 | 10 | 80% |
| 20COW-15 | 3 | 2 | 5 | 22 | 36% |
| 20GAR3-60 | 0 | 3 | 3 | 3 | 0% |
| 20GAR3-62 | 1 | 2 | 3 | 3 | 33% |
| 20GAR3-65 | 0 | 2 | 2 | 2 | 0% |
| 20GAR3-67 | 2 | 1 | 3 | 3 | 67% |
| 20GAR3-69 | 5 | 0 | 5 | 5 | 100% |
| 20GAR3-70 | 1 | 2 | 3 | 3 | 33% |
| 20GAR3-72 | 0 | 3 | 3 | 3 | 0% |
| 20GAR3-73 | 3 | 0 | 3 | 3 | 100% |
| 20GAR3-74 | 0 | 2 | 2 | 2 | 0% |
| 20GAR3-75 | 3 | 0 | 3 | 3 | 100% |
| 20GAR3-78 | 0 | 5 | 5 | 5 | 0% |
| 20GAR3-82 | 2 | 0 | 2 | 2 | 100% |
| 20GAR3-85 | 3 | 4 | 7 | 7 | 43% |
| 20GAR3-86 | 3 | 5 | 8 | 8 | 38% |
| 20GAR3-87 | 2 | 1 | 3 | 3 | 67% |
| 20GAR3-88 | 4 | 5 | 9 | 9 | 44% |
| 20GAR3-89 | 3 | 3 | 6 | 6 | 50% |
| 20GAR3-90 | 0 | 5 | 5 | 5 | 0% |
| 20GAR3-91 | 0 | 3 | 3 | 3 | 0% |
| 20GAR3-92 | 1 | 9 | 10 | 10 | 0% |
| 20GAR3-93 | 0 | 4 | 4 | 4 | 0% |
| 20GAR3-94 | 1 | 4 | 5 | 5 | 20% |
| 20GAR3-95 | 0 | 4 | 4 | 4 | 0% |
| 20GAR3-96 | 0 | 4 | 4 | 4 | 0% |
| 20GAR3-99 | 0 | 6 | 6 | 6 | 0% |
| 20GAR3-100 | 2 | 2 | 4 | 4 | 50% |
| 20GAR3-101 | 0 | 6 | 6 | 6 | 0% |
| 20GAR3-109 | 0 | 2 | 2 | 2 | 0% |
| 20GAR3-110 | 0 | 9 | 9 | 9 | 0% |
| 20GAR3-116 | 0 | 4 | 4 | 4 | 0% |
| 20GAR3-117 | 0 | 4 | 4 | 4 | 0% |
| 20GAR3-117 | 1 | 2 | 3 | 3 | 33% |
| 20GAR3-118 | 0 | 3 | 3 | 3 | 0% |
| 20GAR3-119 | 1 | 6 | 7 | 7 | 14% |
| 20GAR3-120 | 1 | 4 | 5 | 5 | 20% |
| 20GAR3-121 | 1 | 5 | 6 | 6 | 17% |
| 20GAR3-123 | 1 | 2 | 3 | 3 | 33% |
| 20GAR3-125 | 1 | 4 | 5 | 5 | 20% |
| 20GAR3-126 | 0 | 4 | 4 | 4 | 0% |
| 20GAR3-127 | 1 | 8 | 9 | 9 | 11% |
| 20GAR3-128 | 2 | 8 | 10 | 10 | 20% |
| 20GAR3-129 | 0 | 7 | 7 | 7 | 0% |
| 20GAR3-130 | 0 | 6 | 6 | 6 | 0% |
| 20GAR3-131 | 4 | 9 | 13 | 13 | 31% |
| 20TP1B-1001 | 0 | 10 | 10 | 16 | 43% |
| 20TP1B-1002 | 0 | 7 | 7 | 16 | 50% |
| 20TP1B-1003 | 0 | 3 | 3 | 14 | 13% |
| 20TP1B-1004 | 0 | 3 | 3 | 14 | 50% |
| 20TP1B-1005 | 0 | 2 | 2 | 10 | 79% |
| 20TP1B-1006 | 0 | 4 | 4 | 7 | 79% |
| 20TP1B-1007 | 0 | 7 | 7 | 8 | 63% |
| 20TP1B-1008 | 0 | 3 | 3 | 8 | 38% |
| 20TP1B-1009 | 0 | 13 | 13 | 16 | 33% |
| 20TP1B-1010 | 2 | 7 | 9 | 11 | 62% |
| 20TP1B-1011 | 0 | 11 | 11 | 15 | 0% |
| 20TP1B-1012 | 0 | 12 | 12 | 14 | 56% |
| 20TP1B-1013 | 5 | 7 | 12 | 16 | 92% |
| 20TP1B-1014 | 0 | 14 | 14 | 16 | 23% |
| 20TP1B-1027 | 0 | 4 | 4 | 13 | 36% |
| 20TP1B-1028 | 1 | 4 | 5 | 5 | 23% |
| 20TP1B-1029 | 2 | 8 | 10 | 13 | 62% |
| 20TP1B-1030 | 6 | 1 | 7 | 12 | 64% |
| 20TP1B-1031 | 0 | 9 | 9 | 16 | 19% |
| 20TP1B-1032 | 0 | 10 | 10 | 13 | 19% |
| 20TP1B-1033 | 2 | 6 | 8 | 16 | 13% |
| 20TP1B-1035 | 2 | 4 | 6 | 11 | 20% |
| 20TP1B-1036 | 0 | 6 | 6 | 5 | 0% |
| 20TP1B-1037 | 0 | 10 | 10 | 11 | 0% |
| 20TP1B-1038 | 0 | 7 | 7 | 8 | 75% |
| 20TP1B-1039 | 0 | 10 | 10 | 15 | 75% |
| 20TP1B-1040 | 3 | 5 | 8 | 13 | 0% |
| 20TP1B-1041 | 0 | 13 | 13 | 16 | 0% |
| 20TP1B-1042 | 0 | 12 | 12 | 12 | 56% |
| 20TP1C-1022 | 0 | 4 | 4 | 5 | 20% |
| 20TP1C-1023 | 0 | 2 | 2 | 2 | 0% |
| 20TP1C-1025 | 0 | 5 | 5 | 5 | 0% |
| 20TP1C-1027 | 0 | 2 | 2 | 2 | 0% |
| 20TP1C-1028 | 0 | 7 | 7 | 7 | 0% |
| 20TP1C-1029 | 0 | 10 | 10 | 10 | 0% |
| 20TP1C-1030 | 0 | 7 | 7 | 8 | 0% |
| 20TP1C-1031 | 0 | 3 | 3 | 7 | 43% |
| 20TP1C-1034 | 0 | 2 | 2 | 6 | 50% |
| 20TP1D-1 | 3 | 4 | 7 | 22 | 59% |
| 20TP1D-2 | 2 | 0 | 2 | 4 | 100% |
| 20TP1D-4 | 0 | 3 | 3 | 3 | 0% |
| 20TP1D-6 | 3 | 4 | 7 | 17 | 41% |
| 20TP1D-7 | 2 | 0 | 2 | 13 | 77% |
| 20TP1D-10 | 2 | 0 | 2 | 12 | 67% |
| 20TP1D-11 | 3 | 1 | 4 | 9 | 78% |
| 20TP1D-12 | 2 | 2 | 4 | 8 | 75% |
| 20TP1D-14 | 3 | 3 | 6 | 15 | 53% |
| 20TP1D-15 | 3 | 2 | 5 | 16 | 88% |
| 20TP1D-16 | 2 | 0 | 2 | 11 | 73% |
| 20TP1D-17 | 2 | 0 | 2 | 3 | 67% |
| 20TP1D-18 | 3 | 0 | 3 | 12 | 83% |
| 20TP1D-19 | 5 | 0 | 5 | 20 | 90% |
| 20TP1D-21 | 2 | 0 | 2 | 13 | 69% |
| 20TP1D-22 | 0 | 2 | 2 | 9 | 78% |
| 20TP1D-23 | 3 | 3 | 6 | 12 | 50% |
| 20TP1D-24 | 0 | 4 | 4 | 6 | 0% |
| 20TPIC-1019 | 2 | 2 | 4 | 9 | 33% |
| 20TPIC-1032 | 0 | 4 | 4 | 7 | 43% |
| 20TPIC-1035 | 4 | 2 | 6 | 9 | 33% |
| 20TPIC-1036 | 2 | 5 | 7 | 10 | 50% |
| 20TPIC-1037 | 1 | 2 | 3 | 3 | 33% |
| 20TPIC-1038 | 5 | 0 | 5 | 9 | 67% |
| 20TPIC-1039 | 0 | 4 | 4 | 12 | 42% |
| 20TPIC-1040 | 4 | 0 | 4 | 9 | 89% |
| 20TPIC-1042 | 0 | 5 | 5 | 9 | 33% |
| 20TPIC-1050 | 2 | 5 | 7 | 11 | 27% |
| 20TPIC-1053 | 1 | 8 | 9 | 11 | 9% |
| 20VLDY-1001 | 0 | 36 | 36 | 36 | 0% |
| 20VLP2-1 | 0 | 4 | 4 | 4 | 0% |
| 20VLP2-6 | 0 | 2 | 2 | 2 | 0% |
| 20VLP2-7 | 0 | 6 | 6 | 6 | 0% |
| 20VLP2-8 | 0 | 4 | 4 | 4 | 0% |
| 20VLP2-11 | 0 | 6 | 6 | 6 | 0% |
| 20VLP4-6 | 0 | 19 | 19 | 19 | 0% |
| 21TP1A-101 | 9 | 16 | 25 | 25 | 36% |
| 21TP1A-102 | 2 | 6 | 8 | 10 | 20% |
| 21TP1A-103 | 1 | 5 | 6 | 10 | 10% |
| 21TP1A-104 | 2 | 2 | 4 | 10 | 40% |
| 21TP1A-105 | 3 | 18 | 21 | 25 | 12% |
| 21TP1A-106 | 7 | 9 | 16 | 24 | 50% |
| 21TP1A-107 | 2 | 7 | 9 | 16 | 25% |
| 21TP1A-108 | 4 | 2 | 6 | 15 | 53% |
| 21TP1A-109 | 2 | 2 | 4 | 6 | 33% |
| 21TP1A-111 | 0 | 4 | 4 | 6 | 0% |
| 21TP1A-112 | 2 | 1 | 3 | 6 | 50% |
| 21TP1A-113 | 0 | 4 | 4 | 12 | 8% |
| 21TP1A-114 | 2 | 0 | 2 | 12 | 17% |
| 21TP1A-117 | 0 | 5 | 5 | 12 | 0% |
| 21TP1A-118 | 0 | 3 | 3 | 12 | 42% |
| 21TP1A-119 | 1 | 6 | 7 | 12 | 8% |
| 21TP1A-120 | 1 | 1 | 2 | 12 | 8% |
| 21TP1A-121 | 0 | 4 | 4 | 12 | 0% |
| 21TP1A-125 | 0 | 4 | 4 | 12 | 0% |
| 21TP1A-127 | 3 | 0 | 3 | 12 | 50% |
| 21TP1A-128 | 0 | 4 | 4 | 12 | 0% |
| 21TP1A-131 | 0 | 4 | 4 | 12 | 0% |
| 21TP1A-132 | 1 | 2 | 3 | 8 | 13% |
| 21TP1A-133 | 0 | 5 | 5 | 8 | 13% |
| 21TP1A-134 | 0 | 3 | 3 | 12 | 0% |
| 21TP1A-135 | 0 | 4 | 4 | 12 | 0% |
| 21VLP5-1 | 3 | 56 | 59 | 59 | 5% |

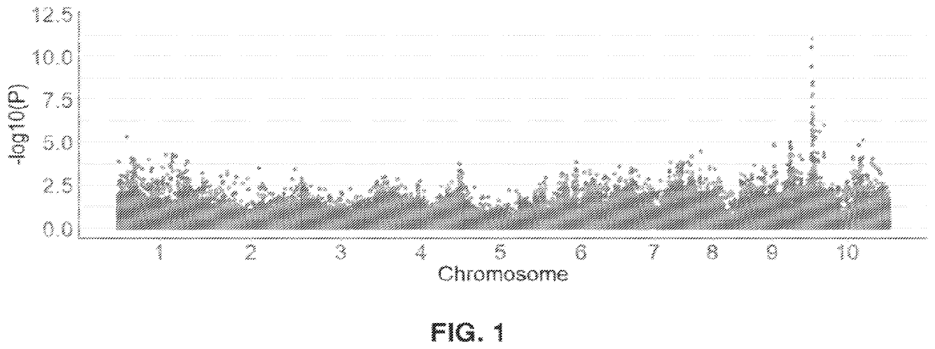

After initial SNP QC, further filtering steps were performed to filter out known low quality SNPs, followed by filtering for missing data <10% and minor allele frequency (>1%) using vcftools (Danecek et al. Bioinformatics 27.15:2156-2158 (2011)). Missing data were subsequently imputed (R package NAM “snpQC” option; Xavier, Alencar, et al. “NAM: association studies in multiple populations.” Bioinformatics 31.23 (2015): 3862-3864). Logistic regression was performed per SNP (n=37,604) using seed lot as random effect in the statistical package R (FIG. 1; Table 2). In total 14 SNP markers were found to be significantly associated with hermaphroditism (Bonferroni multi-test threshold: p=1.32E-06). Of those 14, 13 were located at a locus between positions 5,705,332-8,703,337 bp on chromosome X, with most significantly associated SNP markers 142494_1054190 at position 5,705,332 bp (p=9.21E-12) and 142494_1081185 at position 5,732,323 bp (p=2.91E-11). A second locus at 9.3 Mbp from the first peak contained one SNP marker Cannabis.v1_scf2268-48774_101 at position 17,971,672 bp (p=9.86E-07; Table 2).

TABLE 2
SNP markers significantly associated with hermaphroditism identified in logistic regression of a set of 205 diverse seed lots (n = 1317).

| | | | | | | | | | | Position | Position |
|---|---|---|---|---|---|---|---|---|---|---|---|
| | | | Genotype | | | | | | | left | right |
| | | | associated | | | Abacus | | Left | Right | flanking | flanking |
| Corresp. | SNP | | with | | | reference | | flanking | flanking | marker | marker |
| SEQ ID | marker | | herm. | Ref | Alt | genome | | marker | marker | haplotype | haplotype |
| NO. | name | p-value | resistance | call | call | position | chr | haplotype | haplotype | (bp) | (bp) |
| 1 | 142494_{—} | 9.21E−12 | B, X | G | A | 5,705,332 | X | 142494_{—} | 142494_{—} | 5,696,400 | 5,714,336 |
| | 1054190 | | | | | | | 1045160 | 1063195 | | |
| 2 | 142494_{—} | 2.91E−11 | B, X | G | A | 5,732,323 | X | 142494_{—} | 142494_{—} | 5,725,231 | 5,737,968 |
| | 1081185 | | | | | | | 1074092 | 1086834 | | |
| 3 | 142494_{—} | 2.12E−08 | B, X | A | G | 5,747,057 | X | 142494_{—} | 142494_{—} | 5,737,968 | 5,750,301 |
| | 1095577 | | | | | | | 1086834 | 1098821 | | |
| 4 | 142494_{—} | 7.06E−07 | B, X | C | T | 5,877,981 | X | 142494_{—} | 142494_{—} | 5,860,363 | 5,894,065 |
| | 1226572 | | | | | | | 1208954 | 1242656 | | |
| 5 | 122798_{—} | 3.81E−10 | B, X | A | C | 5,920,712 | X | 142494_{—} | 142494_{—} | 5,894,065 | 5,929,330 |
| | 4414 | | | | | | | 1242656 | 1273038 | | |
| 6 | 142494_{—} | 1.51E−08 | B, X | C | T | 6,053,325 | X | 142494_{—} | 142494_{—} | 6,032,408 | 6,059,371 |
| | 1380839 | | | | | | | 1359921 | 1386938 | | |
| 7 | 142494_{—} | 4.58E−07 | B, X | C | T | 6,181,263 | X | 142494_{—} | 142494_{—} | 6,133,760 | 6,189,246 |
| | 1494517 | | | | | | | 1460180 | 1502498 | | |
| 8 | 142494_{—} | 3.17E−09 | B, X | G | A | 6,186,518 | X | 142494_{—} | 142494_{—} | 6,133,760 | 6,189,246 |
| | 1499770 | | | | | | | 1460180 | 1502498 | | |
| 9 | 142494_{—} | 5.30E−09 | B, X | A | C | 6,192,534 | X | 142494_{—} | 142494_{—} | 6,189,246 | 6,197,726 |
| | 1505786 | | | | | | | 1502498 | 1510978 | | |
| 10 | 142494_{—} | 9.04E−08 | B, X | G | A | 6,261,819 | X | 142494_{—} | 142494_{—} | 6,258,772 | 6,290,824 |
| | 1566767 | | | | | | | 1563720 | 1595682 | | |
| 11 | 142494_{—} | 2.54E−07 | B, X | G | A | 6,285,113 | X | 142494_{—} | 142494_{—} | 6,258,772 | 6,290,824 |
| | 1589970 | | | | | | | 1563720 | 1595682 | | |
| 12 | 142494_{—} | 1.73E−07 | B, X | C | A | 6,695,193 | X | 142494_{—} | 142494_{—} | 6,688,670 | 6,715,018 |
| | 1940317 | | | | | | | 1933794 | 1960221 | | |
| 13 | 142494_{—} | 1.03E−06 | B, X | C | T | 6,961,002 | X | 142494_{—} | 142494_{—} | 6,932,849 | 6,970,023 |
| | 2170761 | | | | | | | 2149596 | 2174824 | | |
| 14 | Cannabis.v1_{—} | 9.86E−07 | B, X | C | T | 17,971,672 | X | Cannabis.v1_{—} | 141972_{—} | 17,900,648 | 17,980,443 |
| | scf2268- | | | | | | | scf4556- | 2448770 | | |
| | 48774_101 | | | | | | | 29548_101 | | | |

First column: SNP marker number; Second column: SNP marker name; Third column, logistic regression p-value; Fourth column, genotype associated with resistance to hermaphroditism: A = homozygous for reference allele, B = homozygous for alternative allele, X = heterozygous; Fifth column, reference allele call; Sixth column, alternative allele call; Seventh column, Abacus reference genome position. Eighth column, Abacus reference genome chromosome; Ninth column, left flanking SNP of haplotype surrounding SNP marker; Tenth column, right flanking SNP of haplotype surrounding SNP marker; Eleventh column, Abacus reference genome position left flanking SNP of haplotype surrounding SNP marker; Twelfth column, Abacus reference genome position right flanking SNP of haplotype surrounding SNP marker. In this context a haplotype surrounding a significantly associated SNP marker consists of the genomic region flanked by the nearest non-significant SNP on either side of the SNP marker.

### F1 Mapping

A set of 11 half-sib F1 populations (n=|12) segregating for hermaphroditic and female flowering accessions was studied for associations with hermaphroditism (Table 3). This set of half-sib seed lots was genotyped using an Illumina bead array. After initial SNP QC, further filtering steps were performed to filter out known low quality SNPs, followed by filtering for missing data <50% and minor allele frequency (>1%) using vcftools (Danecek et al.) In addition, SNPs lacking homozygous alleles of either reference or alternative were removed, resulting in 20,946 SNPs for analysis.

TABLE 3
Seed lots used for association mapping in a set of 11 half-sib F1 populations.
First column: identifier of seed lots (all were grown in a greenhouse); second
column: number of hermaphroditic accessions per seed lot used for mapping;
third column: number of female flowering accessions per seed lot used for
mapping; fourth column: total number of accessions per seed lot used for
mapping; fifth column: total number of accessions evaluated for incidence
of hermaphroditism; sixth column: percentage of hermaphroditic accessions
in total number of accessions evaluated for incidence of hermaphroditism.

| | Hermaphroditic | Female for | Total for | Total | % hermaphroditic in |
|---|---|---|---|---|---|
| Seed lot | for analysis | analysis | analysis | evaluated | total evaluated |
| 20TP1B-1015 | 2 | 10 | 12 | 15 | 13% |
| 20TP1B-1016 | 2 | 4 | 6 | 16 | 0% |
| 20TP1B-1017 | 0 | 9 | 9 | 9 | 0% |
| 20TP1B-1018 | 2 | 8 | 10 | 15 | 31% |
| 20TP1B-1019 | 0 | 13 | 13 | 16 | 36% |
| 20TP1B-1020 | 4 | 4 | 8 | 13 | 40% |
| 20TP1B-1021 | 1 | 7 | 8 | 16 | 7% |
| 20TP1B-1022 | 2 | 12 | 14 | 15 | 0% |
| 20TP1B-1023 | 0 | 15 | 15 | 16 | 38% |
| 20TP1B-1024 | 2 | 2 | 4 | 8 | 50% |
| 20TP1B-1025 | 3 | 10 | 13 | 16 | 20% |

Two Fisher exact tests were performed per SNP using the statistical package R. The first Fisher exact test was based on counts of accessions with homozygous reference allele which were hermaphroditic, accessions with homozygous reference allele which were female, accessions with either homozygous alternate allele or heterozygous which were hermaphroditic, and accessions with either homozygous alternate allele or heterozygous which were female. The second Fisher exact test was based on counts of accessions with homozygous alternate allele which were hermaphroditic, accessions with homozygous alternate allele which were female, accessions which were homozygous reference allele or heterozygous which were hermaphroditic, and accessions which were homozygous reference allele or heterozygous which were female. Subsequently, the most significant p-value of the two tests was recorded. This resulted in 26 significant SNPs based on a Bonferroni multi-test threshold of 2.39E-06.

Subsequently, this set of significant SNPs was narrowed down to SNPs with clear associations, where all or almost all hermaphroditic accessions were homozygous reference or alternative allele and where all or almost all female flowering accessions were the opposite homozygous genotype. For this purpose two ratios were calculated. To all counts the value of 0.0001 was added to deal with divisions involving counts of zero. The first ratio was calculated as the count of hermaphroditic accessions with homozygous reference allele divided by the count of female accessions with homozygous reference allele. The second ratio was calculated as the count of hermaphroditic accessions with homozygous alternate allele divided by the count of female accessions with homozygous alternate allele. SNPs with opposite ratios of hermaphroditic to female for homozygous reference and homozygous alternate alleles were identified as hermaphroditism resistance markers (Tables 4 and 5),

TABLE 4
Counts of accessions in categories used for Fisher Exact Test of 11 half-
sib F1 populations (n = 112) and the tests' p-values.

| | | | | Count | | |
|---|---|---|---|---|---|---|
| | | | | homozygous | Count | |
| | | Count | Count | alternate or | homozygous | P-value |
| Corresp | | homozygous | homozygous | heterozygous | alternate or | homozygous |
| SEQ ID | SNP marker | reference and | reference | and | heterozygous | reference and |
| No. | name | hermaphroditic | and female | hermaphroditic | and female | hermaphroditic |
| 15 | 142169_2381612 | 1 | 12 | 16 | 38 | 1.58E−01 |
| 16 | 141619_63185 | 1 | 40 | 11 | 4 | 1.01E−07 |
| 17 | 142214_442810 | 4 | 62 | 12 | 0 | 4.19E−11 |
| 18 | 142603_7799433 | 6 | 59 | 7 | 13 | 1.02E−02 |

| | | Count | | | | |
|---|---|---|---|---|---|---|
| Count | | homozygous | Count | | | |
| homozygous | Count | reference or | homozygous | P-value | | |
| alternate | homozygous | heterozygous | reference or | homozygous | Herm ratio | Herm ratio |
| and | alternate | and | heterozygous | alternate and | homozygous | homozygous |
| hermaphroditic | and female | hermaphroditic | and female | hermaphroditic | reference | alternate |
| 9 | 0 | 8 | 50 | 5.69E−07 | 0.08 | 90,001 |
| 5 | 0 | 7 | 44 | 2.07E−04 | 0.03 | 50,001 |
| 4 | 0 | 12 | 62 | 1.28E−03 | 0.06 | 40,001 |
| 7 | 0 | 6 | 72 | 3.48E−07 | 0.10 | 70,001 |

First column: Corresponding SEQ ID No.; Second column, SNP marker name; Third column: number of hermaphroditic accessions which are homozygous reference allele; Fourth column: number of female accessions which are homozygous reference allele; Fifth column: number of hermaphroditic accessions which are either homozygous alternate allele or heterozygous; Sixth column: number of female accessions which are either alternate allele or heterozygous; Seventh column: p-value of Fisher Exact test based on columns 3-6; Eighth column: number of hermaphroditic accessions which are homozygous alternate allele; Ninth column: number of female accessions which are homozygous alternate allele; Tenth column: number of hermaphroditic accessions which are either homozygous reference allele or heterozygous; Eleventh column: number of female accessions which are either homozygous reference allele or heterozygous; Twelfth column: p-value of Fisher Exact test based on columns 8-11; Thirteenth column: Ratio of hermaphroditic to female for accessions which are homozygous reference allele; Fourteenth column: Ratio of hermaphroditic to female accessions which are homozygous alternate allele.

TABLE 5
SNP markers significantly associated with hermaphroditism identified
in Fisher Exact Test of a set of 11 half-sib F1 populations (n = 112).

| | | | Genotype | | | Abacus |
|---|---|---|---|---|---|---|
| Corresponding | SNP marker | | associated with | | | reference |
| SEQ ID NO | name | p-value | herm resistance | Ref call | Alt call | genome position |
| 15 | 142169_2381612 | 5.69E−07 | A. X | T | C | 14,810,444 |
| 16 | 141619_63185 | 1.01E−07 | A, X | G | A | 57,051,092 |
| 17 | 142214_442810 | 4.19E−11 | A | C | T | 74,435,555 |
| 18 | 142603_7799433 | 3.48E−07 | A, X | G | A | 5,233,698 |

| | Left flanking marker | Right flanking marker | Position left flanking | Position right flanking |
|---|---|---|---|---|
| chr | haplotype | haplotype | marker haplotype (bp) | marker haplotype (bp) |
| 3 | 142169_2368403 | 142169_2393220 | 14,797,288 | 14,824,861 |
| 3 | 141739_177214 | Cannabis.v1_scf1011- | 56,616,473 | 57,090,960 |
| | | 127620_101 | | |
| 3 | 142214_429649 | 142214_445096 | 74,422,393 | 74,437,841 |
| 4 | 165245_4785 | 142603_7790347 | 5,216,694 | 5,243,017 |

First column: Corresponding SEQ ID No.; Second column, SNP marker name; Third column, Fisher Exact test p-value; Fourth column, genotype associated with resistance to hermaphroditism A = homozygous for reference allele, B = homozygous for alternative allele, X = heterozygous; Fifth column, reference allele call; Sixth column, alternative allele call; Seventh column, Abacus reference genome position. Eighth column, Abacus reference genome chromosome; Ninth column, left flanking SNP of haplotype surrounding SNP marker; Tenth column, right flanking SNP of haplotype surrounding SNP marker; Eleventh column, Abacus reference genome position left flanking SNP of haplotype surrounding SNP marker; Twelfth column, Abacus reference genome position right flanking SNP of haplotype surrounding SNP marker. In this context a haplotype surrounding a significantly associated SNP marker consists of the genomic region flanked by the nearest non-significant SNP on either side of the SNP marker.

### F2 Mapping

QTL mapping was performed with an F2 mapping population (n=294) derived from a cross between a cannabis and a hemp variety which segregated for hermaphroditic and female flowering plants (Table 6). The 294 accessions were evaluated in two consecutive experiments performed in a greenhouse (n=198) and a growth room (n=96), respectively. All 294 accessions were used to create a linkage map, all 96 growth room accessions were used for QTL mapping, however, QTL mapping based on greenhouse data was performed on 182 accessions because some plants were lost due to poor health. Greenhouse data were obtained for up to three clonal replicates per accession (Table 6). A logistic regression was performed on a subset of the greenhouse data for which an accession had either all three replicates developing hermaphroditic flowers or none of the three replicates developing hermaphroditic flowers (=female flowering; n=89; Table 6).

TABLE 6
Growth room and greenhouse grown accessions used for QTL mapping and logistic
regression. First column: location of where plants were grown; second column:
number of accessions with at least one clonal replicate out of up to three
replicates developing hermaphroditic flowers used for QTL mapping; third
column number of accessions with none of the clonal replicates out of up
to three replicates developing hermaphroditic flowers (all replicates producing
female flowers only) used for QTL mapping; fourth column: number of accessions
with all three replicates developing hermaphroditic flowers (used for logistic
regression); fifth column: number of accessions with none of the three replicates
developing hermaphroditic flowers (all three replicates developed female
flowers only; used for logistic regression).

| | Number of | Number of | Number of | |
|---|---|---|---|---|
| | hermaphroditic | female | hermaphroditic | Number of female |
| | accessions for | accessions for | accessions for | accessions for |
| Experiment | QTL mapping | QTL mapping | logistic regression | logistic regression |
| Growth room | 36 | 60 | NA | NA |
| Greenhouse | 74 | 108 | 21 | 68 |

The F2 mapping population segregating for hermaphroditic and female flowering plants (n=294) was genotyped with an Illumina bead array. After initial SNP QC, further filtering steps were performed to filter out known low quality SNPs, SNPs with large numbers of missing values (>50%), linked SNPs (SNPs in 5 kb regions evaluated for LD>0.2) and SNPs with a minimum allele frequency <1% using veftools (Danecek et al.). Subsequently, SNPs deviating from Hardy-Weinberg equilibrium were removed based on a threshold of 1E-06 using plink (Purcell et al. 81.3:559-575 (2007)). After these filtering steps, 7607 array SNPs remained for map construction and QTL analysis. A linkage map was constructed using the F2 mapping population SNP data using the package MSTmap (http://mstmap.org/). QTLs were mapped on this linkage map using the R package QTL (https://rqtl.org/). QTL mapping was performed separately for the greenhouse and growth room data since the two different environments might trigger different stress response genes that could contribute to hermaphroditism. For the greenhouse data an accession was considered hermaphroditic if at least one of the up to three replicates was identified as a hermaphrodite. The accessions in the growth room were not grown in clonal replicates.

One significant hermaphroditism QTL was detected based on the greenhouse data on chromosome 4 (LOD=4.03; 45.708 cM, linkage map haplotype: 7,088,239-7,199,506 bp; p=0.015;). The 1.5 LOD support interval surrounding this QTL was between SNPs 142603_11437550 (16.978 cM) and 142193_3182374 (59.354 cM), which spans a region between 1,092,840 and 77,759,356 bp. Two significant hermaphroditism QTLs were detected based on the growth room data, one on chromosome 1 (LOD=4.74, 60.716 cM; linkage map haplotype: 74,050,935-74,060,284 bp; p=0.014) and another on chromosome 4 (LOD=6.69, 50.0 cM; linkage map haplotype: 35,546,108-58,223,644 bp; p=0.001). The 1.5 LOD support interval surrounding the QTL on chromosome 1 was between SNPs 157_2919163 (48.890 cM) and 138695_7618 (81.788 cM), which spans a region between position 15,983,731 and 78,825,086 on chromosome 1. The 1.5 LOD support interval supporting the QTL on chromosome 4 was between SNPs 142603_4524739 (47.072 cM) and 142193_1790563 (54.232 cM), which spans a region between 8,820,639 and 76,176,165 bp on chromosome 4.

Logistic regression was performed on a subset of greenhouse grown F2 mapping population accessions which had either all three replicates developing hermaphroditic flowers or none of the three replicates developing hermaphroditic flowers; all accessions were genotyped with an Illumina bead array. After initial SNP QC, further filtering steps were performed to filter out known low quality SNPs, followed by filtering for missing data <10% and minor allele frequency (>1%) using veftools (Danecek et al.), resulting in 11,164 array SNPs for input in logistic regression analysis using the statistical package R. Missing data were subsequently imputed (R package NAM “snpQC” option; Xavier, Alencar, et al. “NAM: association studies in multiple populations.” Bioinformatics 31.23 (2015): 3862-3864). The most significantly associated SNP marker with hermapbroditism in this logistic regression analysis was 142603_1004035, located on chromosome 4 at position 12,961,444 (p=9.64E-05; Table 7).

TABLE 7
SNP marker most significantly associated with hermaphroditism identified in
logistic regression of the F2 population grown in a greenhouse (n = 89).

| Corresponding | SNP marker | | Genotype associated | Ref | Alt | Abacus reference |
|---|---|---|---|---|---|---|
| SEQ ID NO | name | p-value | with herm resistance | call | call | genome position |
| 19 | 142603_1004035 | 9.64E−05 | A | G | A | 12,961,444 |

| | Left flanking | Right flanking | Position left flanking | Position right flanking |
|---|---|---|---|---|
| Chromosome | marker haplotype | marker haplotype | marker haplotype (bp) | marker haplotype (bp) |
| 4 | 142603_1032128 | 129356_8438 | 12,923,613 | 13,009,666 |

First column: Corresponding SEQ ID No.; Second column: SNP marker name; Third column, logistic regression p-value; Fourth column, genotype associated with resistance to hermaphroditism: A = homozygous for reference allele, B = homozygous for alternative allele, X = heterozygous; Fifth column, reference allele call; Sixth column, alternative allele call; Seventh column, Abacus reference genome position. Eighth column, Abacus reference genome chromosome; Ninth column, left flanking SNP of haplotype surrounding SNP marker; Tenth column, right flanking SNP of haplotype surrounding SNP marker; Eleventh column, Abacus reference genome position left flanking SNP of haplotype surrounding SNP marker; Twelfth column, Abacus reference genome position right flanking SNP of haplotype surrounding SNP marker. In this context a haplotype surrounding a significantly associated NSP marker consists of the genomic region flanked by the nearest non-significant SNP on either side of the SNP marker.

### SNP Validation

SNP markers identified in the set of 205 diverse seed lots were validated based on beneficial genotypes in the set of 11 half-sib F1 populations as well as based on beneficial genotypes in a validation panel. The validation panel consisted of a set of 88 diverse seed lots that were not used for mapping. These seed lots were evaluated for the formation of hermaphroditic flowers in a greenhouse during 2021-2022. In total 1172 accessions (1-132 accessions per seed lot) were genotyped using an Illumina bead array; 925 of these 1172 accessions were female flowering and 247 accessions were hermaphroditic. Beneficial genotypes were determined by taking the average of hermaphroditism incidence (hermaphroditic accessions were assigned a one and female accessions were assigned a zero) per genotype (homozygous reference allele, heterozygous, homozygous alternative allele). Genotype averages close to zero were identified as beneficial (=plants do not form hermaphroditic flowers). Beneficial genotypes were found to be consistent across the three data sets. The F2 data set was not used for SNP marker validation. The F2 SNP marker was inconclusive in the other three data sets most likely because it was discovered in one genetic background and is possibly restricted to that background only (Table 8).

TABLE 8
Validated SNP markers based on beneficial genotypes associated with female flowering
(=hermaphroditism resistance) in two data sets used for mapping and one validation
set. First column: Corresponding SEQ ID No.; Second column: SNP marker name. Third column:
beneficial genotype associated with female flowering in the diversity panel of 205 diverse
seed lots, (# validated after combining A and X in a single group). Fourth column:
beneficial genotype associated with female flowering in the set of 11 half-sib F1 populations
(B* inferred based on segregation patterns). Fifth column: beneficial genotype associated
with female flowering in the validation panel of 88 diverse seed lots (**Unable to validate
because of low counts of homozygous alternate genotype).

| | | Genotype | Genotype | Genotype |
|---|---|---|---|---|
| | | associated with | associated with | associated with |
| Corresponding | | female flowering | female-flowering | female-flowering in |
| SEQ ID NO | SNP marker name | in diversity panel | in half-sib panel | validation panel |
| 1 | 142494_1054190 | B, X | B, X | B, X |
| 2 | 142494_1081185 | B, X | B, X | B, X |
| 3 | 142494_1095577 | B, X | B, X | B, X |
| 4 | 142494_1226572 | B, X | B, X | B, X |
| 5 | 122798_4414 | B, X | B, X | B, X |
| 6 | 142494_1380839 | B, X | B*, X | B, X |
| 7 | 142494_1494517 | B, X | B, X | B, X |
| 8 | 142494_1499770 | B, X | B, X | B, X |
| 9 | 142494_1505786 | B, X | B, X | B, X |
| 10 | 142494_1566767 | B, X | B*, X | B, X |
| 11 | 142494_1589970 | B, X | B*, X | B, X |
| 12 | 142494_1940317 | B, X | B, X | B, X |
| 13 | 142494_2170761 | B, X | B, X | B, X |
| 14 | Cannabis.v1_scf2268- | B, X | B, X | NA** |
| | 48774_101 | | | |
| 15 | 142169_2381612 | A, X# | A, X | A, X |

### Identification of Candidate Genes

Haplotypes as defined based on logistic regression results for the presence/absence of hermaphroditic flowers in a set of 205 diverse seed lots surrounding the 14 significantly associated SNP markers (Tables 2 and 5) were explored for the presence of candidate genes in the Abacus (Csat_AbacusV2; NCBI assembly accession GCA_025232715.1)) reference genome. A haplotype is the genomic fragment surrounding a significantly associated SNP marker, which is flanked by the nearest non-significant SNP on cither side of the SNP marker.

SNP marker 142494_1054190 is flanked by SNPs 142494_1045160 and 142494_1063195, this haplotype ranges between positions 5,696,400-5,714,336 (bp) on chromosome X. This haplotype contains two candidate genes: a calcium/calcium/calmodulin-dependent Serine/Threonine-kinase (AT2G47010; possibly involved in defense response; 142494_1054190 is 0.25 kb upstream of this gene); Probable elongation factor 1-gamma 2 (EF-1-gamma 2, AT1G57720; has glutathione transferase activity; 142494_1054190 is 7.4 kb upstream of this gene).

SNP marker 142494_1081185 is flanked by SNPs 142494_1074092 and 142494_1086834, this haplotype ranges between positions 5,725,231-5,737,968 (bp) on chromosome X. This haplotype contains one gene: SWI/SNF complex subunit SWI3C (SWITCH/SUCROSE NONFERMENTING 3C (SWI3C)); AT1G21700; regulates flower development in a temperature-dependent manner (Gratkowska-Zmuda et al. International journal of Molecular Sciences 21.3:762 (2020)), interacts with DELLA proteins and modulates gibberellin responses and hormonal cross-talk (Sarnowska et al. Plant physiology 163.1:305-317 (2013)); 142494_1081185 is 1.7 kb downstream of this gene).

SNP marker 142494_1095577 is flanked by SNPs 142494_1086834 and 142494_1098821, this haplotype ranges between positions 5,737,968-5,750,301 (bp) on chromosome X. This haplotype contains one candidate gene: a DPP6 N-terminal domain-like protein (AT1G21680; response to light stimulus (Depuydt et al. bioRxiv (2021)), DELLA up-regulated gene (Cao et al. Plant Physiology 142.2:509-525 (2006)); 142494_1095577 is 2.7 kb downstream of this gene).

SNP marker 142494_1226572 is flanked by SNPs 142494_1208954 and 142494_1242656, this haplotype ranges between positions 5,860,363-5,894,065 bp on chromosome X. This haplotype contains two candidate genes: NAD KINASE 2 (NADK2; AT1G21640; Protein with NAD kinase activity; 142494_1226572 is 11.1 kb upstream of this gene), and Ubinuclein-1 (UBN1; At1g21610; possibly involved replication-independent chromatin assembly; 142494_1226572 is located 0.39 kb upstream of this gene).

SNP marker 122798_4414 is flanked by SNPs 142494_1242656 and 142494_1273038, this haplotype ranges between positions 5,894,065-5,929,330 bp on chromosome X. This haplotype contains two candidate genes: a putative reverse transcriptase (At2g05200; 122798_4414 is located 20.8 kb downstream of this gene), and a Cupredoxin superfamily protein (AT1G72230; 122798_4414 is located 3.3 kb downstream of this gene).

SNP marker 142494_1380839 is flanked by SNPs 142494_1359921 and 142494_1386938, this haplotype ranges between 6,032,408 and 6,059,371 bp on chromosome X. This haplotype contains one gene; Probable protein phosphatase 2C 15 (PP2C15; At1g68410; protein serine phosphatase activity, involved in stress response (Depuydt et al. (2021)); 142494_1380839 is located inside this gene).

SNP markers 142494_1494517 and 142494_1499770 are flanked by SNPs 142494_1460180 and 142494_1502498, this haplotype ranges between 6,133,760-6,189,246 bp on chromosome X. This haplotype contains one gene: Cyclin-A2-2/Cyclin-A2-3 (CYCA2-2/CYCA2-2; At5g11300/At1g15570; mitotic cell cycle gene which contributes to the fine-tuning of local proliferation during plant development (Vanneste et al. EMBO 30.16:3430-3441 (2011)); 142494_1494517 is located 4.0 kb upstream of this gene, 142494_1499770 is located inside this gene).

SNP marker 142494_1505786 is flanked by SNPs 142494_1502498 and 142494_1510978, this haplotype ranges between 6,189,246-6,197,726 bp on chromosome X. This haplotype contains one gene: Serine/threonine protein phosphatase 2A 57 kDa regulatory subunit B′ kappa isoform/2A 59 kDa regulatory subunit B′ gamma isoform/2A 59 kDa regulatory subunit B′ zeta isoform/2A 57 kDa regulatory subunit B′ beta isoform (B′KAPPA/B′GAMMA/B′ZETA/B′BETA; At5g25510/At4g15415/At3g21650/At3g09880; B′GAMMA and B′ZETA together function in growth regulation and high light stress tolerance, involved in controlling reactive oxygen species (ROS) homeostasis and signalling, defense response and high light acclimation upon environmental perturbations (Konert et al. Plant, Cell & Environment 38.12:2641-2651(2015)); 142494_1505786 is located 0.21 kb downstream of this gene).

SNP markers 142494_1566767 and 142494_1589970 are flanked by SNPs 142494_1563720 and 142494_1595682, this haplotype ranges between 6,258,772 and 6,290,824 bp on chromosome X. This haplotype contains one gene: Enhancer of polycomb-like transcription factor protein (EPCR1; AT4G32620; involved in sister chromatid cohesion; 142494_1566767 is located 22.0 kb upstream of this gene, 142494_1589970 is located inside this gene).

SNP marker 142494_1940317 is flanked by SNPs 142494_1933794 and 142494_1960221, this haplotype ranges between 6,688,670-6,715,018 bp on chromosome X. This haplotype contains two genes: an annotated coding sequence with no BLASP hits (142494_1940317 is located inside this gene), and a serine-rich protein-like protein (AT5G25280; potentially involved in response to osmotic stress, light stimulus (Depuydt et al. (2021)); 142494_1940317 is located 0.72 kb downstream of this gene).

SNP marker 142494_2170761 is flanked by SNPs 142494_2149596 and 142494_2174824, this haplotype ranges between 6,932,849-6,970,023 bp on chromosome X. This haplotype contains no genes in the Abacus reference genome, however, 142494_2170761 is 11 kb from uncharacterized protein LOC115695846 on the CBDRx reference genome. SNP marker Cannabis.v1_scf2268-48774_101 is flanked by SNPs Cannabis.v1_scf4556-29548_101 and 141972_2448770, this haplotype ranges between 17,900,648-17,980,443 bp on chromosome X. This haplotype contains two genes: a TTF-type zinc finger protein with HAT dimerization domain-containing protein (LOH3; AT1G19260; ceramide synthase; Cannabis.v1_scf2268-48774_101 is located 9.5 kb downstream of this gene), and Catalase-2 (CAT2; At4g35090; catalyzes the reduction of hydrogen peroxide (H₂O₂), protects against H₂O₂ toxicity caused by high light stress (Zhang et al., International Journal of Molecular Sciences 21.4: 1437(2020)), nitrogen stress (Chu et al. The Plant Cell 33.9:3004-3021(2021)), heat stress (Ono et al. Biochemical and Biophysical Research Communications 534:747-751(2021)), biotic stress (Lv et al. Plant Physiology 181.3:1314-1327(2019)), drought stress (Zou et al. The Plant Cell 27.5:1445-1460 (2015)); Cannabis.v1_scf2268-48774_101 is located inside this gene).

Haplotypes as defined based on Fisher Exact test results for the presence/absence of hermaphroditic flowers in a set of 11 half-sib families, surrounding the 4 significantly associated SNP markers (Table 5) were explored for the presence of candidate genes in the Abacus (Csat_AbacusV2, NCBI assembly accession GCA_025232715) reference genome.

SNP marker 142169_2381612 is flanked by SNPs 142169_2368403 and 142169_2393220, this haplotype ranges between 14,797,288-14,824,861 bp on chromosome 3. This haplotype contains four genes: 1-aminocyclopropane-1-carboxylate synthase 12 (ACS12; At5g51690; catalyze the conversion of S-adenosyl-L-methionine (SAM) into 1-aminocyclopropane-1-carboxylate (ACC), a direct precursor of ethylene, loss of CsACS7 in melon causes andromonoecy (Boualem I 11.5: e0155444 (2016)), ACS2, 5, and 11 interact with RCIIA resulting in cold acclimation (Catala et al. The Plant Cell 26.8:3326-3342 (2014)); 142169_2381612 is located 5.4 kb downstream of this gene), hydroxyproline-rich glycoprotein family protein (AT5G51680; unknown function; 142169_2381612 is located 4.0 kb upstream of this gene), E3 ubiquitin-protein ligase RZFP34 (RXPF34; At5g22920; Promotes abscisic acid (ABA)-induced stomatal closure, reactive oxygen species (ROS) production and drought tolerance (Ding et al. The Plant Cell 27.11:3228-3244 (2015)); 142169_2381612 is located 10.7 kb upstream of this gene), Alpha carbonic anhydrase 2/4/6/7 (ACA2/4/6/7; At2g28210/At4g20990/At4g21000/At1g08080; Reversible hydration of carbon dioxide.; 142169_2381612 is located 14.8 kb downstream of this gene).

SNP marker 141619_63185 is flanked by 141739_177214 and Cannabis.v1_scf1011-127620_101, this haplotype ranges between 56,616,473-57,090,960 bp on chromosome 3. This haplotype contains four genes, but only one gene within 50 kb from SNP marker: Plant calmodulin-binding protein-like protein (AT5G39380).

SNP marker 142214_442810 is flanked by 142214_429649 and 142214_445096, this haplotype ranges between 74,422,393-74,437,841 bp on chromosome 3. This haplotype contains one gene Galactose oxidase/kelch repeat superfamily protein (AT5G50310; unknown function; 142214_442810 is located 8.8 kb downstream of this gene).

SNP marker 142603_7799433 is flanked by 165245_4785 and 142603_7790347, this haplotype ranges between 5,216,694-5,243,017 on chromosome 4. This haplotype contains 2 genes: Lipid phosphate phosphatase epsilon 1/2, chloroplastic (LPPE1/2; At3g50920/At5g66450; lipid biosynthetic process; 142603_7799433 is 4.7 kb upstream of this gene), and Mannan endo-1,4-beta-mannosidase 1/3/4/6/7/P (MAN1/3/4/6/7/P; At1g02310/At3g10890/AT3G10900/At5g01930/At5g66460/A13g30540; MAN1 is involved in response to multiple abiotic and biotic stresses (Depuydt et al. (2021)); 142603_7799433 is located 8.6 kb downstream of this gene).

The haplotype as defined based on logistic regression results for the presence/absence of hermaphroditic flowers in the F2 population, surrounding the significantly associated SNP marker (Table 9) was explored for the presence of candidate genes in the Abacus (Csat_AbacusV2, NCBI assembly accession GCA_025232715) reference genome.

SNP marker 142603_1004035 is flanked by 142603_1032128 and 129356_8438, this haplotype ranges between 12,923,613-13,009,666 bp on chromosome 4. This haplotype contains one gene: 1-aminocyclopropane-1-carboxylate synthase 1/2/4/5/6/7/8/9/11 (ACS1/2/4/5/6/7/8/9/11; At3g61510/At1g01480/A12g22810/At5g65800/At4g11280/At4g26200/A14g37770/At3g49700/At4g08040; catalyze the conversion of S-adenosyl-L-methionine (SAM) into 1-aminocyclopropane-1-carboxylate (ACC), a direct precursor of ethylene, ACS1G gene in cucumber causes female flower production (Zhang et al. The Plant Cell 33.2:306-321(2021)), loss of CsACS7 in melon causes andromonoecy (Boualem et al. PloS One 11.5: e0155444 (2016)), ACS2, 5, 6, and 11 interact with RC11A resulting in cold acclimation (Catalá et al. The Plant Cell 26.8:3326-3342 (2014)); 142603_1004035 is located 1.2 kb downstream of this gene).

### Identification of Candidate Gene Subset

Next, all validated SNP markers were explored further in the validation set for a subset which in combination improved predictability of female flowering (=hermaphroditism resistance) as compared to individual markers. In total, eight genotype combinations of the SNP markers 142494_1081185, Cannabis.v1_scf2268-48774_101, and 142169_2381612 were identified. None of these genotype combinations were observed in hermaphroditic accessions. It is therefore expected that for a plant to produce hermaphroditic flowers that at least one of these three markers needs to display the detrimental genotype.

TABLE 9
Combinations of three main hermaphroditism SNP marker genotypes
that are predictive of female flowering (=hermaphroditism resistance:
A = homozygous reference allele, X = heterozygous,
B = homozygous alternate allele). First column: genotype for SNP
marker 142494_1081185; Second column: genotype for SNP marker
Cannabis.v1_scf2268-48774_101; Third column: genotype for
SNP marker 142169_2381612.

| | Cannabis.v1_{—} | |
|---|---|---|
| 142494_{—} | scf2268- | 142169_{—} |
| 1081185 | 48774_101 | 2381612 |
| B | B | A |
| B | B | X |
| X | B | A |
| X | B | X |
| B | B | A |
| B | B | X |
| X | B | A |
| X | B | X |

Based on these findings, SNP marker 142494_1081185, SNP marker Cannabis.v1_scf2268-48774_101, and SNP marker 142169_2381612 were further investigated.

SNP marker 142494_1081185 is flanked by SNPs 142494_1074092 and 142494_1086834. This haplotype ranges between positions 5,725,231-5,737,968 (bp) on chromosome X, and contains SWI/SNF complex subunit SWI3C, which was identified as a candidate gene for further study. SWI3C coding sequence (CDS) is located between 5,725,473-5,730,545 bp on chromosome X of the Abacus reference genome. Its CBDRx (version cs10) homolog is LOC115699778 and is located between 99,767,658-99,773,384 bp on chromosome X of the CBDRx reference genome.

SNP marker Cannabis.v1_scf2268-48774_101 is flanked by SNPs Cannabis.v1_scf4556-29548_101 and 141972_2448770. This haplotype ranges between 17,900,648-17,980,443 bp on chromosome X and contains catalase-2, which was identified as a candidate gene for further study. CAT2 coding sequence (CDS) is located between 17,970,499-17,972,014 bp on chromosome X of the Abacus reference genome. Its CBDRx (version cs10) homolog is LOC115713783 and is located between 70,264,547-70,268,696 bp on chromosome X of the CBDRx reference genome.

SNP marker 142169_2381612 is flanked by SNPs 142169_2368403 and 142169_2393220. This haplotype ranges between 14,797,288-14,824,861 bp on chromosome 3 and contains 1-aminocyclopropane-1-carboxylate synthase 12 (ACS12), which was identified as a candidate gene for further study. ACS12 coding sequence (CDS) is located between 14,803,123-14,804,961 bp on chromosome 3 of the Abacus reference genome. Its CBDRx (version cs10) homolog is LOC115709518 and is located between 36,551,233-36,555,484 bp on chromosome 3 of the CBDRx reference genome.

### Example 2

### Discovery of Genes Associated with Hermapbroditism

### Gene Sequencing and Expression Analysis

RNA was extracted from leaf tissue from accessions differing for marker genotypes known to be predictive of female flowering (=hermaphroditism resistance; Nucleospin RNA Plant and Fungi kit, Macherey-Nagel; Table 10). After concentration adjustment and treatment with DNAse the RNA was used directly for RT-PCR (OneTaq® One-Step RT-PCR Kit, New England Biolabs). Sanger sequencing of CDS was performed based on RT-PCR product (NEB PCR® Cloning Kit; New England Biolabs). Genomic DNA (extracted from leaf tissue with a NucleoMag Plant DNA Kit, Macherey-Nagel) was used to sequence the end and 3′UTR of each gene. Sanger sequencing based on cloned RT-PCR or PCR product was performed for areas with high levels of heterozygosity, Sanger sequencing based on RT-PCR or PCR product without cloning was performed for areas with low levels of heterozygosity. Primers for amplification and sequencing are listed in Table 13.

TABLE 10
Marker genotypes (A = homozygous reference allele,
X = heterozygous, B = homozygous alternate
allele) and nucleotides for causative SNPs in accessions used for gene
sequencing and expression analysis of the three candidate gencs
(NA = not applicable since gene was not sequenced in accession).
First column: accession name;
Second column: genotype for SNP marker 142494_1081185 which has SWI3C in its
haplotype, beneficial genotype is B or X;
Third column: nucleotide of causative SNP at position 728 bp of the CDS of SWI3C;
Fourth column: nucleotide of causative SNP at position 1193 bp of the CDS of SWI3C;
Fifth column: genotype of SNP marker Cannabis,v1_scf2268-48774_101 which contains
CAT2 in its haplotype, beneficial genotype is B or X;
Sixth column: nucleotide of causative SNP at position 338 bp of the 3′UTR;
Seventh column: nucleotide sequence of causative indel located at position
394-400 bp of the 3′UTR of accessions with the beneficial
Cannabis.v1_scf2268-48774_101 SNP marker genotype;
Eight column: genotype of SNP marker 142169_2381612 which contains ACS12
in its haplotype, beneficial genotype is A or X;
Ninth column: nucleotide sequence of causative indel located at position
1500-1508 bp of the CDS of accessions with the detrimental genotype for
SNP marker 142169_2381612;
Tenth column: nucleotide sequences of causative indel located at position
25-28 bp of the 3′UTR of accessions with the beneficial genotype for SNP
marker 142169_2381612;
Eleventh column: nucleotide of causative SNP at position 31 bp of the
3′UTR of accessions with the beneficial genotype for SNP marker 142169_2381612.

| | SWI3C | SWI3C CDS | SWI3C CDS | CAT2 | CAT2 3′UTR |
|---|---|---|---|---|---|
| Accession | marker | (728 bp) | (1193 bp) | marker | (338 bp) |
| 21TP1B-1-1 | B | G | C | A | T |
| 21TCV1-4-5 | NA | NA | NA | B | C |
| 21LCV1-1-13 | A | A | T | | T |
| 21TP1B-21-1 | B | G | C | B | C |
| 20TP1B-1015-5 | NA | NA | NA | NA | NA |
| 20TP1B-1016-6 | NA | NA | NA | NA | NA |
| 20TP1B-1020-1 | NA | NA | NA | NA | NA |
| Abacus | A | A | T | A | T |
| | | | ACS12 | ACS12 | |
| CAT2 3′UTR | ACS12 | ACS12 CDS | 3′UTR | 3′UTR | |
| (394-400 bp) | marker | (1500-1508 bp) | (25-28 bp) | (31 bp) | |
| ------- | NA | NA | NA | NA | |
| CTGATAT | NA | NA | NA | NA | |
| ------- | NA | NA | NA | NA | |
| CTGATAT | NA | NA | NA | NA | |
| NA | B | TACCGAAAG | ---- | T | |
| NA | B | TACCGAAAC | ---- | T | |
| NA | A | --------- | TTTT | C | |
| ------- | A | --------- | TTTT | C | |

Alignment of Sanger sequenced fragments was performed per accession for each gene. The resulting consensus sequences were subsequently aligned per gene. Functional CDS were translated to protein sequences, which were subsequently aligned with Arabidopsis thaliana protein sequence to identify functional domains. Functional domains were explored further in the protein sequence alignments for amino acid substitutions that would alter these domains.

### SWI3C

Alignment of SWI3C CDS and protein sequences of accessions that contain the beneficial genotype for SNP marker 142494_1081185 (21TP1B-1-1 and 21TP1B-21-1; SEQ ID NO: 30, 31, 34 and 35; Table 10 and 14) and accessions that contain the detrimental genotype for this marker (21LCV1-1-13 and Abacus; SEQ ID NO: 32, 33, 36, and 37; Table 10 and 14) revealed two amino acid changing substitutions that were in common between the two accessions with the beneficial genotype (homozygous alternate allele) for SNP marker 142494_1081185 (21TP1B-1-1 and 21TP1B-21-1) and that were absent in the two accessions with the detrimental genotype (homozygous reference allele) for SNP marker 142494_1081185 (21LCV1-1-13 and Abacus).

The first amino acid substitution is a K (Lysine, observed in 21LCV1-1-13 and Abacus; SEQ ID NO: 36 and 37) to R (Arginine, observed in 21TPIB-1-1 and 21TPIB-21-1; SEQ ID NO: 34 and 35; Table 14) amino acid substitution at position 242 from the start codon caused by an A to G nucleotide substitution (K242R; 21LCV1-1-13 and Abacus: A, SEQ ID NO: 32 and 33; 21TP1B-1-1 and 21TP1B-21-1: G, SEQ ID NO: 30 and 31; Table 14) at CDS position 728 bp from the start codon (located at position 51 bp in SEQ ID NO: 38; Table 14).

The second amino acid changing substitution is an M (Methionine, observed in 21LCV1-1-13 and Abacus; SEQ ID NO: 36 and 37) to T (Threonine, observed in 21TP1B-1-1 and 21TP1B-21-1; SEQ ID NO: 34 and 35) amino acid substitution at position 398 from the start codon caused by a T to C nucleotide substitution (M398T; 21LCV1-1-13 and Abacus: T, SEQ ID NO: 32 and 33; 21TP1B-1-1 and 21TP1B-21-1: C, SEQ ID NO: 30 and 31) at CDS position 1193 bp from the start codon (located at position 51 bp in SEQ ID NO: 39).

The first amino acid substitution is located in the SWIRM domain based on alignment of cannabis amino acid sequences with the Arabidopsis thaliana SWI3C homolog (Uniprot ID Q9XI07). The K amino acid observed for Abacus and 21LCV1-1-13 was conserved in Arabidopsis thaliana, which is a hermaphroditic species. The Swi3 SWIRM domain binds to DNA and mononucleosomes and is important for complex assembly; substitution mutants in this domain show impaired DNA-binding activity (Da, Guoping, et al. “Structure and function of the SWIRM domain, a conserved protein module found in chromatin regulatory complexes.” Proceedings of the National Academy of Sciences 103.7 (2006): 2057-2062.). The second amino acid substitution is located in the zinc finger domain based on alignment of cannabis amino acid sequences with the Arabidopsis thaliana SWI3C homolog (Uniprot ID Q9XI07). Arabidopsis thaliana has a V where Abacus, and 21LCV1-1-13 have an M. This amino acid substitution is located 16 amino acids from the fourth Zn²⁺ binding site, located at amino acid position 384 in cannabis. The zinc finger domain enables proteins to bind with DNA, RNA, and other proteins, plays a role in regulating processes such as development, and can function as transcriptional repressor in the acclimation response of plants to different environmental stress conditions (Ciftci-Yilmaz, S., and R. Mittler. “The zinc finger network of plants.” Cellular and Molecular Life Sciences 65.7 (2008): 1150-1160.).

In Arabidopsis, the SWI3C core subunit of the switch/sucrose nonfermenting (SWI/SNF) chromatin remodeling complex (Arabidopsis homolog AT1G21700) reportedly interacts with DELLA proteins and modulates gibberellin responses. Inhibition of gibberellic acid (GA) production feminizes male flowers, whereas exogenous application of GA on female plants can cause development of male flowers (Sarath, G., and H. Y. Mohan Ram. “Comparative effect of silver ion and gibberellic acid on the induction of male flowers on female Cannabis plants.” Experientia 35.3 (1979): 333-334.; West, Nicholas W., and Edward M. Golenberg. “Gender-specific expression of GIBBERELLIC ACID INSENSITIVE is critical for unisexual organ initiation in dioecious Spinacia oleracea.” New Phytologist 217.3 (2018): 1322-1334.). Without being bound to any particular theory, it is possible that the amino acid substitutions in the SWIRM and zinc finger domains observed in the accessions with the beneficial genotype for SNP marker 142494_1081185 cause reduced GA production, whereas the amino acids associated with the detrimental genotype of this marker result in increased GA production.

### CAT2

Alignment of CAT2 CDS and protein sequences of accessions differing for the Cannabis.v1_scf2268-48774_101 SNP marker genotype (21TPIB-21-1 and 21TCV1-4-5 have the beneficial genotype; 21LCV1-1-13 and 21TP1B-1-1, and Abacus have the detrimental genotype; Table 10) revealed no amino acid substitutions in the sequenced CDS. However, alignment of the 3′UTR region revealed a T to C substitution at position 338 bp from the start of the 3′UTR (21TP1B-21-1 and 21TCV1-4-5: C, SEQ ID NO: 40 and 41; 21LCV1-1-13, 21TP1B-1-1, and Abacus: T, SEQ ID NO: 42, 43, and 44; located at position 51 bp in SEQ ID NO: 45: Table 14).

In addition, a seven nucleotide (CTGATAT) insertion between positions 394-400 bp was observed for the two accessions with the beneficial Cannabis.v1_scf2268-48774_101 SNP marker genotype (21TPIB-21-1 and 21TCV1-4-5; SEQ ID NO: 40 and 41; Table 14). This insertion was absent in the four accessions with the detrimental Cannabis.v1_scf2268-48774_101 SNP marker genotype (21LCV1-1-13, 21TP1B-1-1, and Abacus; SEQ ID NO: 42, 43, and 44; deletion located between positions 51-56 bp in SEQ ID NO: 46; Table 14).

Arabidopsis homolog CAT2 (At4g35090) catalyzes the reduction of hydrogen peroxide (H₂O₂), and protects against H₂O₂ toxicity caused by high light stress (Zhang, Shan, et al. “BAK1 mediates light intensity to phosphorylate and activate catalases to regulate plant growth and development.” International journal of molecular sciences 21.4 (2020): 1437), nitrogen stress (Chu, Xiaoqian, et al. “HBI transcription factor-mediated ROS homeostasis regulates nitrate signal transduction.” The Plant Cell 33.9 (2021): 3004-3021), heat stress (Ono, Masaaki, et al. “CATALASE2 plays a crucial role in long-term heat tolerance of Arabidopsis thaliana.” Biochemical and Biophysical Research Communications 534 (2021): 747-751), biotic stress (Lv, Tianxiao, et al. “The calmodulin-binding protein IQM1 interacts with CATALASE2 to affect pathogen defense.” Plant physiology 181.3 (2019): 1314-1327), and drought stress (Zon, Jun-Jie, et al. “Arabidopsis CALCIUM-DEPENDENT PROTEIN KINASE8 and CATALASE3 function in abscisic acid-mediated signaling and H₂O₂ homeostasis in stomatal guard cells under drought stress.” The Plant Cell 27.5 (2015): 1445-1460.). Hydrogen peroxide is a key signaling molecule in plant stem cell regulation, including the stem cell fate for flowering transition (Huang, Xiaozhen, et al. “ROS regulated reversible protein phase separation synchronizes plant flowering.” Nature Chemical Biology 17.5 (2021): 549-557.). Higher catalase activity has been reported in male as compared to female cannabis plants (Elena, T. R. U. Ú., et al. “Biochemical differences in Cannabis sativa L. depending on sexual phenotype.” J. Appl. Genet 43.4 (2002): 451-462.). Without being bound to any particular theory, it is possible that the nucleotide substitution and the seven nucleotide insertion observed in accessions with the beneficial genotype for the Cannabis.v1_scf2268-48774_101 SNP marker modify the 3′UTR so that it decreases CAT2 expression and enzyme activity. Increased H₂O₂ levels may direct stem cell fate towards female flower structures. On the other hand, the nucleotide and seven nucleotide deletion observed in accessions with the detrimental genotype for the Cannabis.v1_scf2268-48774_101 SNP marker modify the 3′UTR may increase CAT2 expression and enzyme activity, resulting in decreased H₂O₂ levels that direct stem cell fate towards male flower structures.

### ACS12

For ACS12 two accessions with the beneficial (homozygous reference allele) genotype for SNP marker 142169_2381612 (20TP1B-1020-1 and Abacus; Table 10) were compared with two accessions with the detrimental (homozygous alternate allele) genotype for this SNP marker (20TP1B-1016-6 and 20TP1B-1015-5; Table 10). Three of these accessions (20TP1B-1020-1, 20TP1B-1016-6, and 20TP1B-1015-5) were part of the set of half-sib populations used to map the 142169_2381612 SNP marker. CDS and protein sequences for these accessions were compared with the CBDRx reference genome ACS12 homolog LOC115709518 sequence (CBDRx has the detrimental genotype for SNP marker 142169_2381612). Alignment of Abacus and CDBRx ACS12 CDS revealed that amino acid substitutions were located at the end of the CDS and the beginning of the 3′UTR. As a result, the end of the CDS and the beginning of the 3′UTR were sequenced in the three accessions differing for SNP marker 142169_2381612 (20TP1B-1020-1, 20TP1B-1016-6, and 20TP1B-1015-5; SEQ ID NO: 47, 48, 49, 51, 52, 53, 55, 56, and 57; Table 14) and compared with Abacus CDS, protein, and 3′UTR sequences (SEQ ID NO: 50, 54, and 58; Table 14).

The two accessions with the detrimental genotype for SNP marker 142169_2381612 (20TP1B-1016-6 and 20TPIB-1015-5) have an insertion of three amino acids at positions 501-503 of the protein sequence (SEQ ID NO: 52 and 53). This insertion is TET (Threonine, Glutamic Acid, Threonine) for 20TP1B-1016-6 (SEQ ID NO: 52) and TES (Threonine, Glutamic Acid, Serine) for 20TP1B-1015-5 (SEQ ID NO: 53), however this insertion is absent in accessions with the beneficial genotype for SNP marker 142169_2381612: 20TP1B-1020-1 (SEQ ID NO: 51) and Abacus (SEQ ID NO: 54). The three amino acid insertion is caused by a nine nucleotide (TACCGAAAC) insertion for 20TP1B-1016-6 at CDS positions 1500-1508 bp from the start codon (SEQ ID NO: 48) and a nine nucleotide (TACCGAAAG) insertion for 20TP1B-1015-5 at CDS positions 1500-1508 bp from the start codon (SEQ ID NO: 49). These nine nucleotides are absent in 20TP1B-1020-1 (SEQ ID NO: 47) and Abacus (SEQ ID NO: 50; the deletion is located in Abacus at positions 51-59 bp of SEQ ID NO: 59).

ACS12 is a 1-Aminocyclopropane-1-carboxylic acid synthase (ACS) protein. ACS proteins are rate-limiting enzymes in endogenous ethylene biosynthesis. External application of ethylene can increase femaleness in cucurbits (Wang, Zhongyuan, et al. “Systematic genome-wide analysis of the ethylene-responsive ACS gene family: Contributions to sex form differentiation and development in melon and watermelon.” Gene 805 (2021): 145910.). The two amino acid substitutions and the insertion observed in the two hermaphroditic accessions containing the detrimental genotype for SNP marker 142169_2381612 are located in the alpha-fold region of the ACS12 gene based on alignment with the Arabidopsis thaliana ACS12 homolog (Uniprot ID Q8GYY0). Without being bound by any particular theory, it is possible the substitutions/insertion reduce the functionality of the ACS12 gene and therefore its effect on ethylene production, resulting in hermaphroditic flowers.

The two accessions with the detrimental genotype for SNP marker 142169_2381612 (20TP1B-1016-6 and 20TP1B-1015-5) have a four nucleotide deletion between positions 24 and 25 bp from the start of the 3′UTR (SEQ ID NO: 56 and 57, respectively), whereas the two accessions with the beneficial genotype for SNP marker 142169_2381612, 20TP1B-1020-1 and Abacus, have an insertion of four nucleotides (TTTT) at position 25-28 bp from the start of the 3′UTR (SEQ ID NO: 55 and 58, respectively; located at positions 51-54 bp in Abacus SEQ ID NO: 60). In addition, the two accessions with the detrimental genotype for SNP marker 142169_2381612 (20TP1B-1016-6 and 20TP1B-1015-5) have a C to T nucleotide substitution at position 27 bp from the start of the 3′UTR (20TP1B-1016-6 and 20TP1B-1015-5: T; SEQ ID NO: 56 and 57) corresponding with position 31 bp from the start of the 3′UTR for 20TP1B-1020-1 and Abacus (20TP1B-1020-1 and Abacus: C; SEQ ID NO: 55 and 58; located at position 51 bp in Abacus SEQ ID NO: 61).

Both the four nucleotide deletion and the C to T nucleotide substitution observed in the two accessions with the detrimental genotype for SNP marker 142169_2381612 (20TP1B-1016-6 and 20TP1B-1015-5) are located in a predicted microRNA (miRNA) binding site for miRNA ath-miR5021. This miRNA binding site was found in genes that were downregulated in response to stress (Munusamy, Prabhakaran, ct al. “De novo computational identification of stress-related sequence motifs and microRNA target sites in untranslated regions of a plant translatome.” Scientific reports 7.1 (2017): 1-14.). In the two accessions with the beneficial genotype for SNP marker 142169_2381612 (Abacus and 20TP1B-1020-1) the target site is a stretch of 20 nucleotides (TTTTTTCTTTCTTCTTCTCA) located between positions 25-44 bp from the start of the 3′UTR (SEQ ID NO: 58 and 55, respectively), whereas in the two accessions with the detrimental genotype for SNP marker 142169_2381612 (20TP1B-1016-6 and 20TP1B-1015-5) the target site is a stretch of 20 nucleotides (TTTATTTTTTCTTCTTCTCA) located between positions 21-40 bp from the start of the 3′UTR (SEQ ID NO: 56 and 57, respectively). Although ath-miR5021 has a perfect match for its 5′ end to its target site in all four cannabis accessions, this miRNA's 3′ end has in total three mismatches with the target site in the four cannabis sequences, however, mismatches with 20TP1B-1020-1 and Abacus (containing the beneficial genotype for SNP marker 142169_2381612) are at position 5, 7, and 8 from the 3′ end of the miRNA (SEQ ID NO: 55 and 58, respectively), whereas mismatches with 20TP1B-16-6 and 20TP1B-1015-5 (containing the detrimental genotype for SNP marker 142169_2381612) are at position 4, 5, and 8 from the 3′ end of the miRNA (SEQ ID NO: 56 and 57, respectively), Without being bound to any particular theory, stress-induced reduction of ACS12 expression through miRNA binding may reduce ethylene production and increase hermaphroditic flowers in plants that contain the four nucleotide deletion and the C to T nucleotide substitution as observed in the two hermaphroditic accessions with the detrimental genotype for SNP marker 142169_2381612 (20TP1B-1016-6 and 20TP1B-1015-5). Conversely, in cannabis plants with the four nucleotide insertion, lacking the C to T nucleotide substitution as observed in the two accessions with the beneficial genotype for SNP marker 142169_2381612 (20TP1B-1020-1 and Abacus) ACS12 expression may be higher as a result of stress, due to weaker miRNA binding.

Hermaphroditism is a complex phenotype, influenced by a number of environmental conditions/stress. However, the combination of beneficial genotypes in SWI3C, CAT2, and ACS12 ensures that cannabis plants are resistant to the formation of hermaphroditic flowers regardless of genetic background or environmental stress that is experienced. Plants containing the combination of beneficial genotypes in SWI3C, CAT2, and ACS12 described are not known to exist in nature.

TABLE 11
a listing of single nucleotide polymorphism markers, which are located at position 26 of each
respective sequence. First column: Corresponding SEQ ID No; Second column: SNP marker name: Third
column: Abacus reference genome (Csat_AbacusV2; NCBI assembly accession GCA_025232715.1)
sequence containing the reference allele of the SNP marker at position 26 bp.

| SEQ ID NO | Description/SNP ID | Sequence |
|---|---|---|
| 1 | 142494_1054190 | CTGAATTCTTCGTCTTCTAGTTCCTGTACACTTCA |
| | | CACTCACGGGGCAACT |
| 2 | 142494_1081185 | TCATCAGTCCTATTAGAATACGTTTGCCCACTTTC |
| | | TTCCTTTTGGGATCAT |
| 3 | 142494_1095577 | AAGAATAGGCTTTGTGTTAGTTAGAATTAGTTACT |
| | | TTGCTCTCACGGTTGA |
| 4 | 142494_1226572 | AGCCAAACGGTTGCCGCCCTCATATCTATTAATTA |
| | | AGGCTTCTCAATTAGC |
| 5 | 122798_4414 | CTATTTAATTATATTCTCTATATTAACAGGGCTTGA |
| | | AGAGGACTACACTCT |
| 6 | 142494_1380839 | CGTCGTCGGGTGGTTGACTGCATTTCTAAACTCCG |
| | | AGCTAGATTTTCATAG |
| 7 | 142494_1494517 | TCTCTGACACATGCAAGGACGATTCCTTGACTTAA |
| | | AAATAGGCAATGGAAG |
| 8 | 142494_1499770 | GATTACATTTCATTTTAGGACTCCTGATTGAATTG |
| | | AATTGAATACCCTTTT |
| 9 | 142494_1505786 | AGTATAGAAAATCGTTAATCCATTTAATGGCCCCT |
| | | TCCCATAAACTTAGCC |
| 10 | 142494_1566767 | GATCCAATCCAATTCATGCCATATTGGAAGGATAT |
| | | AGAGTGCATATACATG |
| 11 | 142494_1589970 | TACCAGACAGTGAAGATGTTGTGAAGAAAGCAGA |
| | | ACCCTTGAGTGACAAAC |
| 12 | 142494_1940317 | CCCAACCGCCCAAAGTAACACCTGTCATATTACAA |
| | | AATCAATTAATTATTA |
| 13 | 142494_2170761 | GAAAAAGGGAAAGAACGTGATATAGCTAGTTGAT |
| | | TGGAGGAATAGGTTTGT |
| 14 | Cannabis.v1_scf2268- | CCAGACAGGTACATGCATATGTGGTCATTTTACCA |
| | 48774_101 | TGTCAAAAACAGTATT |
| 15 | 142169_2381612 | GATTTCTGACTCAGAGCCTTTGGTATTAAAAGTTC |
| | | AGGATGTAGCGTGATC |
| 16 | 141619_63185 | CACTAAAATGTTGTACACTAAATTTGACATCATTA |
| | | ATAGTGTTGAATACTG |
| 17 | 142214_442810 | AATTAAAATAATCATATATTCATTOCCAACTTOCTT |
| | | TCTTCTTTAACTTTC |
| 18 | 142603_7799433 | ACAAAAGATAATATCGGACATAGAGGATAGAGGT |
| | | ATGGAGCCGAACCTTTG |
| 19 | 142603_1004035 | CATATATCTTTTTCTTTGTCATGCTGTTATGTTATG |
| | | TAAAGAAAAATTGAA |

TABLE 12
provides a listing of the single nucleotide polymorphism markers of Table 11
with additional 50 bp of flanking sequence. The single nucleotide polymorphisms
are located at position 51 of each respective sequence.
First column: Corresponding SEQ ID No;
Second column: SNP marker name;
Third column: Abacus reference genome sequence containing the reference
allele of the SNP marker at position 51 bp.

| SEQ ID | Description/SNP | |
|---|---|---|
| NO | ID | Sequence |
| 62 | 142494_1054190 | TTCCTCGTTTTTCACCAACCGAGTACTGAATTCTTCGTCTTCTAGTT |
| | | CCTGTACACTTCACACTCACGGGGCAACTGTCCAAATCCGAAAGCT |
| | | AGCTCAGG |
| 63 | 142494_1081185 | CATCACGGGCCAACTCTCGACCTCGTCATCAGTCCTATTAGAATAC |
| | | GTTTGCCCACTTTCTTCCTTTTGGGATCATGTTCGGCCTTGATGGGT |
| | | TGTTGGTC |
| 64 | 142494_1095577 | AAAAGCATCTGCAAAGGTGATGTTAAAGAATAGGCTTTGTGTTAGT |
| | | TAGAATTAGTTACTTTGCTCTCACGGTTGAGTTAGTTAGGTTGTTTA |
| | | GATTTTGT |
| 65 | 142494_1226572 | AACCCGTACAGTTCCCTACTTTAAAAGCCAAACGGTTGCCGCCCTC |
| | | ATATCTATTAATTAAGGCTTCTCAATTAGCCGGTGATTGACGGGTA |
| | | TTCTACTCT |
| 66 | 122798_4414 | GAATATAATGTTTACCTAATTTGGACTATTTAATTATATTCTCTATA |
| | | TTAACAGGGCTTGAAGAGGACTACACTCTAGAGAAGAATGTAAAA |
| | | GCTTTTGAG |
| 67 | 142494_1380839 | AAGACTTCAGTTAGATTCAATTGTTCGTCGTCGGGTGGTTGACTGC |
| | | ATTTCTAAACTCCGAGCTAGATTTTCATAGAATGTTCTTTTTTTTTC |
| | | CTTGACAT |
| 68 | 142494_1494517 | TATGAAAATGAAGCATAATTTCTTGTCTCTGACACATGCAAGGACG |
| | | ATTCCTTGACTTAAAAATAGGCAATGGAAGACCCTAACTCAGACA |
| | | AGTGGAGCAA |
| 69 | 142494_1499770 | ACCAATCATCAAGAAGAAAAATTATGATTACATTTCATTTTAGGAC |
| | | TCCTGATTGAATTGAATTGAATACCCTTTTCCTCCTCAGCTAGAGC |
| | | GAAGGCCAT |
| 70 | 142494_1505786 | GAATTTGAAAAATCAAGTACTATCTAGTATAGAAAATCGTTAATCC |
| | | ATTTAATGGCCCCTTCCCATAAACTTAGCCAGGCAATGAAGTGTAC |
| | | ATGGTAAAA |
| 71 | 142494_1566767 | GTTCATCTTGGAACTCGTTCGTGGGGATCCAATCCAATTCATGCCA |
| | | TATTGGAAGGATATAGAGTGCATATACATGGTCATCTTCTTGGACC |
| | | GACAGAAAC |
| 72 | 142494_1589970 | AAACGACGTGGAAATAAAAAATTTGTACCAGACAGTGAAGATGTT |
| | | GTGAAGAAAGCAGAACCCTTGAGTGACAAACCTTCTACGACACAC |
| | | AATACTACCCG |
| 73 | 142494_1940317 | TTCATCGAAAATCCAGCTTAGACTTCCCAACCGCCCAAAGTAACAC |
| | | CTGTCATATTACAAAATCAATTAATTATTAAATAATTGACAGCCAA |
| | | ACTAACCAA |
| 74 | 142494_2170761 | TGCGGCATGACGAATGAGAACCAAAGAAAAAGGGAAAGAACGTG |
| | | ATATAGCTAGTTGATTGGAGGAATAGGTTTGTGACAAAGATATAG |
| | | GAGATTGCATTG |
| 75 | Cannabis.v1_scf2 | TGGTGTGAGATACCGATCTTGGGCACCAGACAGGTACATGCATAT |
| | 268-48774_101 | GTGGTCATTTTACCATGTCAAAAACAGTATTGAAGTGTTTGGTTTC |
| | | ATTTGTATGC |
| 76 | 142169_2381612 | TCTGGGTTAATCTTATAGCGTTGATGATTTCTGACTCAGAGCCTTTG |
| | | GTATTAAAAGTTCAGGATGTAGCGTGATCGAGTTTGGAAAAAAAT |
| | | AGTTGCAGC |
| 77 | 141619_63185 | CACTCTAATAGCATACCAAATTATACACTAAAATGTTGTACACTAA |
| | | ATTTGACATCATTAATAGTGTTGAATACTGAATCTATACCAATTTA |
| | | ATTTTAGTG |
| 78 | 142214_442810 | AAGTACTAGCTGACATTCTCTAACTAATTAAAATAATCATATATTC |
| | | ATTCCCAACTTCCTTTCTTCTTTAACTTTCCATTGCCAATGGACTAA |
| | | TAATTTTT |
| 79 | 142603_7799433 | AAATCCGTTAGACCTTACCGTCATTACAAAAGATAATATCGGACAT |
| | | AGAGGATAGAGGTATGGAGCCGAACCTTTGACAAGAAAACGTCTA |
| | | TTTGGCCACA |
| 80 | 142603_1004035 | GAGTCTACACTTCTAGCCACACATGCATATATCTTTTTCTTTGTCAT |
| | | GCTGTTATGTTATGTAAAGAAAAATTGAACTTGTCTAGAGGAAAA |
| | | AGTGGCTCT |

TABLE 13
provides a list of primers; primers with SEQ
ID NOs 20-27 are intended for amplification
of CDS based on RNA, primers marked with SEQ
ID NO. 28-29 (marked with *) are intended for
amplification of genomic DNA. First column:
corresponding SEQ ID No; Second column:
primer name; Third column: primer sequence.

| SEQ ID | Description/ | |
|---|---|---|
| NO | SNP ID | Sequence |
| 20 | SWI3CC_AF | AGGAAGACGAGGAGGAGGAC |
| 21 | SWI3CC_AR | GGATTCCCTGAGGCATTGTA |
| 22 | SWI3CCfr1_AR | TCTGTCCAACTTTCCCCATC |
| 23 | SWI3CCff2_AF | TGCTCTGATTGCTTCCATGA |
| 24 | CAT2C AF | CGACCATCGAGTGCTTACAA |
| 25 | CAT2C_AR | AGCACCCAATTCCTGAGCTT |
| 26 | CAT2C_2AF | AGCCCACCTGTGGTGTTAAG |
| 27 | CAT2C 2AR | TGGCCAGATTTGTGTTGTGT |
| 28 | ACS12K_AF* | GGCTGGTTTCGTTGTTGTTT |
| 29 | ACS12K_AR* | GAAATGGGCATCAAGAGGAA |

provides additional sequence information.
First column: corresponding SEQ ID No;
Second column: sequence description,
*Incomplete CDS, coordinates in brackets show the start and end of the
sequence as compared to the Abacus reference genome homologous CDS,
S** = G or C;
Third column: sequences (genomic DNA, CDS, or protein sequences as
indicated in the second column description of the
sequences).

| SEQ ID | Description/SNP | |
|---|---|---|
| NO | ID | Sequence |
| 30 | 2ITPIB-1-1 | TTGGAGAATGTCTCGCACGGGCAGCTTCAGGCTTTATCGGCTGTGC |
| | SW3C CDS | CTCCTGATTGCCCTAGTTTGGATCAGGATAGGTCCGATGGGGGAA |
| | (397-2279 bp)* | ATTCGGCTTATGTGGTCACTCCGCCTCCGATTATGGAAGGCCGCGG |
| | | CGTTGTGAAGCGTTTTGGGACTCGGTCTCATGTCGTTCCAATGCAT |
| | | TCGGATTGGTTTTCACCTGCTACAGTGCATAGACTAGAGAGACAA |
| | | GTGGTGCCACATTTTTTCTCAGGAAAATCACCAGACCATACTCCTG |
| | | AGAAATACATGGAATGTAGGAACTATGTTGTTGCCAAATACATGG |
| | | AGAATCCAGAAAGACGGTTCACTGTGTCTGATTTCGTGGGAGTGA |
| | | TACCTGGTCTTGAAACTGAAGATTTAAGTCGAATAGTTCGGTTTCT |
| | | TGATCACTGGGGAATAATCAATTATTGTGCTGCTGTATCAAGTCAT |
| | | GAGCCATGGAATAGCAGTTCCTACTTAAAGGAGGAGTCAAATGGT |
| | | GAGATTCACGTGCCATCTGCTGCGTTAAAGTCCATTGATAGCTTAA |
| | | TCAAGTTTGAAAAGCCCAAATGCAAGCTCAAGGCAGCTGATGTTT |
| | | ATTCATCCCTTTCAAGTCATGACAACAATGTCTCTGATTTGGACAA |
| | | CAGAATTCGTGAGCATTTATCTGAAAATCGTTGCAATAGTTGTTCT |
| | | CGATCTCTACCCACTGTATGCTATCAGTCACACAAGGAGGTGGATA |
| | | TAATGCTATGCTCTGATTGCTTCCATGATGGAAGGTATGTGATTGG |
| | | TCACTCAAGCTTAGATTTTACGAGGATGGATTCAACAAAAGATTAT |
| | | GGCGATCCAGATGGGGAAAGTTGGACAGATCAAGAAACGTTGTTG |
| | | CTACTTGAGGCAGTGGAAATGTACAATGAGAACTGGAATGAAATT |
| | | GCAGAGCATGTTGGAACTAAGTCTAAAGCCCAATGCATTCTTCATT |
| | | TCCTTCGCCTGCCTGTGGAAGATGGCCTTCTGGAAAATATTGAAGT |
| | | TCCTGGGAATCCTATATCATCCAGTATGTCAAATGGAGCTGTTCAT |
| | | GGACAAATAATTGCAAATGGGGATTCAGCAGGATCATGCCAACAA |
| | | GATGCTGATTTTGAAAGCAGATTCCCTTTTGCCAATTCTGGGAATC |
| | | CGGTCATGTCCCTGGTTGCCTTTCTGGCTTCTTCGGTTGGCCCAAG |
| | | AGTCGCTGCAGCATGTGCACATGCATCCTTGGCAGCGTTGTCGGAG |
| | | GAAAGTAGTTTATCTGCATCGGAGAACCTGTTACATAAGGAAGGA |
| | | CCAGGACAGAGTAATAGGATGAACTCAGAGATTATTCATGGTAGA |
| | | GATGGTGGCCCTCAGGGAGAGATTGCAAATTCAGCTCACCATAAA |
| | | GCTGTTGCTTCAAGGGGTCAAAATGAAGGGGCTACTCCATTATCA |
| | | GCAGAAAAGGTTAAAGCTGCTGCTAAAGTTGGCTTGGCTGCTGCA |
| | | GCAACGAAGGCCAAATTGTTTGCTGATCATGAAGAACGAGAAATC |
| | | CAGAGACTCTCTGCCAATATTATAAATCATCAGCTGAAGCGATTGG |
| | | AGCTGAAGTTAAAGCAATTTGCTGAAGTGGAAACATTCCTAATGA |
| | | AAGAATGTGAACAAGTGGAGAGGACAAGACAGAGACTTTTGGCTG |
| | | AGCGATCTCGTTTTATGGCAGCACAAATGGGAGCTAGCGGGATCA |
| | | CTTCTATGACTCTACCAGGAGCACCTGGTCCTTCCATGGTTAACAA |
| | | CTCAGGTAACAATAGACAACAACTTATGTCAGCCGCACCTTCACA |
| | | ACCGAGCATATCAGGATACAACAACAATCACCAACCACAACAGGT |
| | | TCATCCCCACATGCCATTCAGGCCTCGGCAGCAAATGTTTGCTATG |
| | | GGGCCAAGGATGCCGCT |
| 31 | 21TPIB-21-1 | CAGGATCGTGAGGATGATTCAGAGGATGGTGTCCAACCTAACCCT |
| | SWI3C CDS | CAATCTGGCTCTTCCGCACCCGATCCCTCTTCTTCCTCTCAAGAGA |
| | (142-2279 bp)* | GTGAGGTCTCTCCCGACGGTGTGGTTCGTTGCTCTGATTTTCCACCT |
| | | GTCATGAGGCGCATCTTGAATCGCCCTCACCCTTCCGTTATGGCCA |
| | | TTGTTGCTTTGGAGCGAGCTAATGAGTTTGGGGAAAGCAAGGGTC |
| | | AAGGTCAGGGAAGCTCTTTAATGTTTTTGGAGAATGTCTCGCACGG |
| | | GCAGCTTCAGGCTTTATCGGCTGTGCCTCCTGATTGCCCTAGTTTG |
| | | GATCAGGATAGGTCCGATGGGGGAAATTCGGCTTATGTGGTCACT |
| | | CCGCCTCCGATTATGGAAGGCCGCGGCGTTGTGAAGCGTTTTGGG |
| | | ACTCGGTCTCATGTCGTTCCAATGCATTCGGATTGGTTTTCACCTGC |
| | | TACAGTGCATAGACTAGAGAGACAAGTGGTGCCACATTTTTTCTCA |
| | | GGAAAATCACCAGACCATACTCCTGAGAAATACATGGAATGTAGG |
| | | AACTATGTTGTTGCCAAATACATGGAGAATCCAGAAAGACGGTTC |
| | | ACTGTGTCTGATTTCGTGGGAGTGATACCTGGTCTTGAAACTGAAG |
| | | ATTTAAGTCGAATAGTTCGGTTTCTTGATCACTGGGGAATAATCAA |
| | | TTATTGTGCTGCTGTATCAAGTCATGAGCCATGGAATAGCAGTTCC |
| | | TACTTAAAGGAGGAGTCAAATGGTGAGATTCACGTGCCATCTGCT |
| | | GCGTTAAAGTCCATTGATAGCTTAATCAAGTTTGAAAAGCCCAAAT |
| | | GCAAGCTCAAGGCAGCTGATCTTTATTCATCCCTTTCAAGTCATGA |
| | | CAACAATGTCTCTGATTTGGACAACAGAATTCGTGAGCATTTATCT |
| | | GAAAATCGTTGCAATAGTTGTTCTCGATCTCTACCCACTGTATGCT |
| | | ATCAGTCACACAAGGAGGTGGATATAATGCTATGCTCTGATTGCTT |
| | | CCATGATGGAAGGTATGTGATTGGTCACTCAAGCTTAGATTTTACG |
| | | AGGATGGATTCAACAAAAGATTATGGCGATCCAGATGGGGAAAGT |
| | | TGGACAGATCAAGAAACGTTGTTGCTACTTGAGGCAGTGGAAATG |
| | | TACAATGAGAACTGGAATGAAATTGCAGAGCATGTTGGAACTAAG |
| | | TCTAAAGCCCAATGCATTCTTCATTTCCTTCGCCTGCCTGTGGAAG |
| | | ATGGCCTTCTGGAAAATATTGAAGTTCCTGGGAATCCTATATCATC |
| | | CAGTATGTCAAATGGAGCTGTTCATGGACAAATAATTGCAAATGG |
| | | GGATTCAGCAGGATCATGCCAACAAGATGCTGATTTTGAAAGCAG |
| | | ATTCCCTTTTGCCAATTCTGGGAATCCGGTCATGTCCCTGGTTGCCT |
| | | TTCTGGCTTCTTCGGTTGGCCCAAGAGTCGCTGCAGCATGTGCACA |
| | | TGCATCCTTGGCAGCGTTGTCGGAGGAAAGTAGTTTATCTGCATCG |
| | | GAGAACCTGTTACATAAGGAAGGACCAGGACAGAGTAATAGGAT |
| | | GAACTCAGAGATTATTCATGGTAGAGATGGTGGCCCTCAGGGAGA |
| | | GATTGCAAATTCAGCTCACCATAAAGCTGTTGCTTCAAGGGGTCAA |
| | | AATGAAGGGGCTACTCCATTATCAGCAGAAAAGGTTAAAGCTGCT |
| | | GCTAAAGTTGGCTTGGCTGCTGCAGCAACGAAGGCCAAATTGTTT |
| | | GCTGATCATGAAGAACGAGAAATCCAGAGACTCTCTGCCAATATT |
| | | ATAAATCATCAGCTGAAGCGATTGGAGCTGAAGTTAAAGCAATTT |
| | | GCTGAAGTGGAAACATTCCTAATGAAAGAATGTGAACAAGTGGAG |
| | | AGGACAAGACAGAGACTTTTGGCTGAGCGATCTCGTTTTATGGCA |
| | | GCACAAATGGGAGCTAGCGGGATCACTTCTATGACTCTACCAGGA |
| | | GCACCTGGTCCTTCCATGGTTAACAACTCAGGTAACAATAGACAA |
| | | CAACTTATGTCAGCCGCACCTTCACAACCGAGCATATCAGGATAC |
| | | AACAACAATCACCAACCACAACAGGTTCATCCCCACATGCCATTC |
| | | AGGCCTCGGCAGCAAATGTTTGCTATGGGGCCAAGGATGCCGCT |
| 32 | 2ILCV1-1-13 | CAGGATCGTGAGGATGATTCAGAGGATGGTGTCCAACCTAACCCT |
| | SWI3C CDS | CAATCTGGCTCTTCCGCACCCGATCCCTCTTCTTCCTCTCAAGAGA |
| | (142-2291 bp)* | GTGAGGTCTCTCCCGACGGTGTGGTTCGTTGCTCTGATTTTCCACCT |
| | | GTCATGAGGCGCATCTTGAATCGCCCTCACCCTTCCGTTATGGCCA |
| | | TTGTTGCTTTGGAGCGAGCTAATGAGTTTGGGGAAAGCAAGGGTC |
| | | AAGGTCAGGGAAGCTCTTTAATGTTTTTGGAGAATGTCTCGCACGG |
| | | GCAGCTTCAGGCTTTATCGGCTGTGCCTCCTGATTGCCCTAGTTTG |
| | | GATCAGGATAGGTCCGATGGGGGAAATTCGGCTTATGTGGTCACT |
| | | CCGCCTCCGATTATGGAAGGCCGCGGCGTTGTGAAGCGTTTTGGG |
| | | ACTCGGTCTCATGTCGTTCCAATGCATTCGGATTGGTTTTCACCTGC |
| | | TACAGTGCATAGACTAGAGAGACAAGTGGTGCCACATTTTTTCTCA |
| | | GGAAAATCACCAGACCATACTCCTGAGAAATACATGGAATGTAGG |
| | | AACTATGTTGTTGCCAAATACATGGAGAATCCAGAAAAACGGTTC |
| | | ACTGTGTCTGATTTCGTGGGAGTGATACCTGGTCTTGAAACTGAAG |
| | | ATTTAAGCCGAATAGTTCGGTTTCTTGATCACTGGGGAATAATCAA |
| | | TTATTGTGCTGCTGTATCAAGTCATGAGCCATGGAATAGCAGTTCC |
| | | TACTTAAAGGAGGAGTCAAATGGTGAGATTCACGTGCCATCTGCT |
| | | GCATTAAAGTCCATTGATAGCTTAATCAAGTTTGAAAAGCCCAAAT |
| | | GCAAGCTCAAGGCAGCTGATGTTTATTCATCCCTTTCAAGTCATGA |
| | | CAACAATGTCTCTGATTTGGACAACAGAATTCGTGAGCATTTATCT |
| | | GAAAATCGTTGCAATAGTTGTTCTCGATCTCTACCCACTGTATGCT |
| | | ATCAGTCACACAAGGAGGTGGATATAATGCTATGCTCTGATTGCTT |
| | | CCATGATGGAAGGTATGTGATTGGTCACTCAAGCTTAGATTTTATG |
| | | AGGATGGATTCAACAAAAGATTATGGCGATCCAGATGGGGAAAGT |
| | | TGGACAGATCAAGAAACGTTGTTGCTACTTGAGGCAGTGGAAATG |
| | | TACAATGAGAACTGGAATGAAATTGCAGAGCATGTTGGAACTAAG |
| | | TCTAAAGCCCAATGCATTCTTCATTTCCTTCGCCTGCCTGTGGAAG |
| | | ATGGCCTTCTGGAAAATATTGAAGTTCCTGGGAATCCTATATCATC |
| | | CAGTATGTCAAATGGAGCTGTTCATGGACAAATAATTGCAAATGG |
| | | GGATTCAGCAGGATCATGCCAACAAGATGCTGATTTTGAAAGCAG |
| | | ATTCCCTTTTGCCAATTCTGGGAATCCGGTCATGTCCCTGGTTGCCT |
| | | TTCTGGCTTCTTCGGTTGGCCCAAGAGTCGCTGCAGCATGTGCACA |
| | | TGCATCCTTGGCAGCATTGTCGGAGGAAAGTAGTTTATCTGCATCG |
| | | GAGAACCTGTTACATAAGGAAGGACCAGGACAGAGTAATAGGAT |
| | | GAACTCAGAGATTATTCATGGTAGAGATGGTGGCCCTCAGGGAGA |
| | | GATTGCAAATTCAGCTCACCATAAAGCTGTTGCTTCAAGGGGTCAA |
| | | AATGAAGGGGCTACTCCATTATCAGCAGAAAAGGTTAAAGCTGCT |
| | | GCTAAAGTTGGCTTGGCTGCTGCAGCAACAAAGGCCAAATTGTTT |
| | | GCTGATCATGAAGAACGAGAAATCCAGAGACTCTCTGCCAATATT |
| | | ATAAATCATCAGCTGAAGCGATTAGAGCTGAAGTTAAAGCAATTT |
| | | GCTGAAGTGGAAACATTCCTAATGAAAGAWTGTGAACAAGTGGA |
| | | GAGGACAAGACAGAGACTTTTGGCTGAGCGATCTCGTTTTATGGC |
| | | AGCACAAATGGGAGCTAGCGGGATCACTTCTATGACTCTACCAGG |
| | | AGCACCTGGTCCTTCCATGGTTAACAACTCAGGTAACAATAGACA |
| | | ACAACTTATGTCAGCCGCACCTTCACAACCGAGCATATCAGGATA |
| | | CAACAACAATCACCAACCACAACAGGTTCATCCCCACATGCCATT |
| | | CAGGCCTCGGCAGCAAATGTTTGCTATGGGGCCAAGGATGCCGCT |
| | | TGGTGCCATACA |
| 33 | Abacus SWI3C | ATGCCAGCTTCTTCTCCTTCAGATGGGGGGGGCAAATGGAGGAAG |
| | CDS (1-2403 | CGGCGGAGAGAACCCCAAATCAATCGGAGGCTGAAGCAGGAAGA |
| | bp) | CGAGGAGGAGGACGAAGACGACGCCGTTCCAGATGAGAATCTTGA |
| | | ACAACAACAGGATCGTGAGGATGATTCAGAGGATGGTGTCCAACC |
| | | TAACCCTCAATCTGGCTCTTCCGCACCCGATCCCTCTTCTTCCTCTC |
| | | AAGAGAGTGAGGTCTCTCCCGACGGTGTGGTTCGTTGCTCTGATTT |
| | | TCCACCTGTCATGAGGCGCATCTTGAATCGCCCTCACCCTTCCGTT |
| | | ATGGCCATTGTTGCTTTGGAGCGAGCTAATGAGTTTGGGGAAAGC |
| | | AAGGGTCAAGGTCAGGGAAGCTCTTTAATGTTTTTGGAGAATGTCT |
| | | CGCACGGGCAGCTTCAGGCTTTATCGGCTGTGCCTCCTGATTGCCC |
| | | TAGTTTGGATCAGGATAGGTCCGATGGGGGAAATTCGGCTTATGT |
| | | GGTCACTCCGCCTCCGATTATGGAAGGCCGCGGCGTTGTGAAGCG |
| | | TTTTGGGACTCGGTCTCATGTCGTTCCAATGCATTCGGATTGGTTTT |
| | | CACCTGCTACAGTGCATAGACTAGAGAGACAAGTGGTGCCACATT |
| | | TTTTCTCAGGAAAATCACCAGACCATACTCCTGAGAAATACATGG |
| | | AATGTAGGAACTATGTTGTTGCCAAATACATGGAGAATCCAGAAA |
| | | AACGGTTCACTGTGTCTGATTTCGTGGGAGTGATACCTGGTCTTGA |
| | | AACTGAAGATTTAAGCCGAATAGTTCGGTTTCTTGATCACTGGGGA |
| | | ATAATCAATTATTGTGCTGCTGTATCAAGTCATGAGCCATGGAATA |
| | | GCAGTTCCTACTTAAAGGAGGAGTCAAATGGTGAGATTCACGTGC |
| | | CATCTGCTGCATTAAAGTCCATTGATAGCTTAATCAAGTTTGAAAA |
| | | GCCCAAATGCAAGCTCAAGGCAGCTGATGTTTATTCATCCCTTTCA |
| | | AGTCATGACAACAATGTCTCTGATTTGGACAACAGAATTCGTGAG |
| | | CATTTATCTGAAAATCGTTGCAATAGTTGTTCTCGATCTCTACCCA |
| | | CTGTATGCTATCAGTCACACAAGGAGGTGGATATAATGCTATGCTC |
| | | TGATTGCTTCCATGATGGAAGGTATGTGATTGGTCACTCAAGCTTA |
| | | GATTTTATGAGGATGGATTCAACAAAAGATTATGGCGATCCAGAT |
| | | GGGGAAAGTTGGACAGATCAAGAAACGTTGTTGCTACTTGAGGCA |
| | | GTGGAAATGTACAATGAGAACTGGAATGAAATTGCAGAGCATGTT |
| | | GGAACTAAGTCTAAAGCCCAATGCATTCTTCATTTCCTTCGCCTGC |
| | | CTGTGGAAGATGGCCTTCTGGAAAATATTGAAGTTCCTGGGAATCC |
| | | TATATCATCCAGTATGTCAAATGGAGCTGTTCATGGACAAATAATT |
| | | GCAAATGGGGATTCAGCAGGATCATGCCAACAAGATGCTGATTTT |
| | | GAAAGCAGATTCCCTTTTGCCAATTCTGGGAATCCGGTCATGTCCC |
| | | TGGTTGCCTTTCTGGCTTCTTCGGTTGGCCCAAGAGTCGCTGCAGC |
| | | ATGTGCACATGCATCCTTGGCAGCATTGTCGGAGGAAAGTAGTTTA |
| | | TCTGCATCGGAGAACCTGTTACATAAGGAAGGACCAGGACAGAGT |
| | | AATAGGATGAACTCAGAGATTATTCATGGTAGAGATGGTGGCCCT |
| | | CAGGGAGAGATTGCAAATTCAGCTCACCATAAAGCTGTTGCTTCA |
| | | AGGGGTCAAAATGAAGGGGCTACTCCATTATCAGCAGAAAAGGTT |
| | | AAAGCTGCTGCTAAAGTTGGCTTGGCTGCTGCAGCAACAAAGGCC |
| | | AAATTGTTTGCTGATCATGAAGAACGAGAAATCCAGAGACTCTCT |
| | | GCCAATATTATAAATCATCAGCTGAAGCGATTAGAGCTGAAGTTA |
| | | AAGCAATTTGCTGAAGTGGAAACATTCCTAATGAAAGAATGTGAA |
| | | CAAGTGGAGAGGACAAGACAGAGACTTTTGGCTGAGCGATCTCGT |
| | | TTTATGGCAGCACAAATGGGAGCTAGCGGGATCACTTCTATGACTC |
| | | TACCAGGAGCACCTGGTCCTTCCATGGTTAACAACTCAGGTAACA |
| | | ATAGACAACAACTTATGTCAGCCGCACCTTCACAACCGAGCATAT |
| | | CAGGATACAACAACAATCACCAACCACAACAGGTTCATCCCCACA |
| | | TGCCATTCAGGCCTCGGCAGCAAATGTTTGCTATGGGGCCAAGGA |
| | | TGCCGCTTGGTGCCATACAGTCACCATCATCTGCTCCAAATGTCAT |
| | | GTACAATGCCTCAGGGAATCCCCAACCTAGTATGAATCATCCGAT |
| | | GTTAAGGCCAGTACCTGGTACTAGCTCTGGTTTGGGGTGA |
| 34 | 21TP1B-1-1 | LENVSHGQLQALSAVPPDCPSLDQDRSDGGNSAYVVTPPPIMEGRGV |
| | SW3C protein | VKRFGTRSHVVPMHSDWFSPATVHRLERQVVPHFFSGKSPDHTPEKY |
| | sequence (133- | MECRNYVVAKYMENPERRFTVSDFVGVIPGLETEDLSRIVRFLDHWG |
| | 759 AA) | TINYCAAVSSHEPWNSSSYLKEESNGEIHVPSAALKSIDSLIKFEKPKCK |
| | | LKAADVYSSLSSHDNNVSDLDNRIREHLSENRCNSCSRSLPTVCYQSH |
| | | KEVDIMLCSDCFHDGRYVIGHSSLDFTRMDSTKDYGDPDGESWTDQE |
| | | TLLLLEAVEMYNENWNEIAEHVGTKSKAQCILHFLRLPVEDGLLENIE |
| | | VPGNPISSSMSNGAVHGQIANGDSAGSCQQDADFESRFPFANSGNPV |
| | | MSLVAFLASSVGPRVAAACAHASLAALSEESSLSASENLLHKEGPGQ |
| | | SNRMNSEIIHGRDGGPQGEIANSAHHKAVASRGQNEGATPLSAEKVK |
| | | AAAKVGLAAAATKAKLPADHEEREIQRLSANINHQLKRLELKLKQF |
| | | AEVETFLMKECEQVERTRQRLLAERSRFMAAQMGASGITSMTLPGAP |
| | | GPSMVNNSGNNRQQLMSAAPSQPSISGYNNNHQPQQVHPHMPFRPR |
| | | QQMFAMGPRMP |
| 35 | 2ITPIB-21-1 | QDREDDSEDGVQPNPQSGSSAPDPSSSSQESEVSPDGVVRCSDFPPVM |
| | SWI3C protein | RRILNRPHPSVMAIVALERANEFGESKGQGQGSSLMFLENVSHGQLQ |
| | sequence (48- | ALSAVPPDCPSLDQDRSDGGNSAYVVTPPPIMEGRGVVKRFGTRSHV |
| | 759 AA) | VPMHSDWFSPATVHRLERQVVPHFFSGKSPDHTPEKYMECRNYVVA |
| | | KYMENPERRFTVSDFVGVIPGLETEDLSRIVRFLDHWGIINYCAAVSS |
| | | HEPWNSSSYLKEESNGEIHVPSAALKSIDSLIKFEKPKCKLKAADVYSS |
| | | LSSHDNNVSDLDNRIREHLSENRQNSCSRSLPTVCYQSHKEVDIMLCS |
| | | DCFHDGRYVIGHSSLDFTRMDSTKDYGDPDGESWTDQETLLLLEAVE |
| | | MYNENWNEIAEHVGTKSKAQCILHFLRLPVEDGLLENIEVPGNPISSS |
| | | MSNGAVHGQIANGDSAGSCQQDADFESRFPFANSGNPVMSLVAFLA |
| | | SSVGPRVAAACAHASLAALSEESSLSASENLLHKEGPGQSNRMNSEII |
| | | HGRDGGPQGEIANSAHHKAVASRQQNEGATPLSAEKVKAAAKVGLA |
| | | AAATKAKLFADHEEREIQRLSANIINHQLKRLELKLKQFAEVETFLMK |
| | | ECEQVERTRQRLLAERSRFMAAQMGASGITSMTLPGAPGPSMVNNSG |
| | | NNRQQLMSAAPSQPSISGYNNNHQPQQVHPHMPFRPRQQMFAMGPR |
| | | MP |
| 36 | 21LCV1-1-13 | QDREDDSEDGVQPNPQSGSSAPDPSSSSQESEVSPDGVVRCSDFPPVM |
| | SWI3C protein | RRILNRPHPSVMAIVALERANEFGESKGQGQGSSLMFLENVSHGQLQ |
| | sequence (48- | ALSAVPPDCPSLDQDRSDGGNSAYVVTPPPIMEGRGVVKRFGTRSHV |
| | 763 AA) | VPMHSDWFSPATVHRLERQVVPHFFSGKSPDHTPEKYMECRNYVVA |
| | | KYMENPEKRFTVSDFVGVIPGLETEDLSRIVRFLDHWGIINYCAAVSS |
| | | HEPWNSSSYLKEESNGEIHVPSAALKSIDSLIKFEKPKCKLKAADVYSS |
| | | LSSHDNNVSDLDNRIREHLSENRCNSCSRSLPTVCYQSHKEVDIMLCS |
| | | DCFHDGRYVIGHSSLDFMRMDSTKDYGDPDGESWTDQETLLLLEAV |
| | | EMYNENWNEIAEHVGTKSKAQCILHFLRLPVEDGLLENIEVPGNPISSS |
| | | MSNGAVHGQIIANGDSAGSCQQDADFESRFPFANSGNPVMSLVAFLA |
| | | SSVGPRVAAACAHASLAALSEESSLSASENLLHKEGPGQSNRMNSEII |
| | | HGRDGGPQGEIANSAHHKAVASRGQNEGATPLSAEKVKAAAKVGLA |
| | | AAATKAKLFADHEEREIQRLSANUINHQLKRLELKLKQPAEVETFLMK |
| | | XCEQVERTRQRLLAERSRFMAAQMGASGITSMTLPGAPGPSMVNNS |
| | | GNNRQQLMSAAPSQPSISGYNNNHQPQQVHPHMPFRPRQQMFAMGP |
| | | RMPLGAI |
| 37 | Abacus SWI3C | MPASEDRRGKWKRKKRGGLSAARKPKQEEEDMEEEDEENNNNNNE |
| | protein sequence | EMDDVENADELQQNGGATPDPGLGIGEVVEDSGSRISDFPAVVKRVV |
| | (1-800 AA) | IRPHASVMAVVAAERAGLIGETRGQGSLPALENISFGQLQALSTVPAD |
| | | SLDLERSDGSSSAYVISPPPIMDGEGVVKRFGDL VHVLPMHSDWFAP |
| | | NTVDRLERQVVPQFFSGKSPNHTPESYMEFRNAIVSKYVENPEKTLTI |
| | | SDCQGLVDGVDIEDFARVFRFLDHWGIINYCATAQSHPGPLRDVSDV |
| | | REDTNGEVNVPSAALTSIDSLIKFDKPNCRHKGGEVYSSLPSLDGDSP |
| | | DLDIRIREHLCDSHCNHCSRPLPTVYFQSQKKGDILLCCDCFHHGRFV |
| | | VGHSCLDFVRVDPMKFYGDQDGDNWTDQETLLLLEAVELYNENWV |
| | | QIADHVGSKSKAQCILHFLRLPVEDGLLDNVEVSGVTNTENPTNGYD |
| | | HKGTDSNGDLPGYSEQGSDTEIKLPFVKSPNPVMALVAFLASAVGPR |
| | | VAASCAHESLSVLSEDDRMKSEGMQGKEASLLDGENQQQDGAHKTS |
| | | SQNGAEAQTPLPQDKVMAAFRAGLSAAATKAKLFADHEEREIQRLSA |
| | | NIVNHQLKRMELKLKQFAEIETLLMKECEQVEKTRQRFSAERARMLS |
| | | ARFGSPGGISPQTNNLQGMSLSTGGNNINSLMHQQHQQQQASATSQP |
| | | SHIPGFSNNPQVQAQMHFMARQQQQQQQQQQQQQQAFSFGPRLPLN |
| | | AIQTNAGSTASPNVMFGNNQLNNPAAAGAASINQPSFSHPMVRSSTG |
| | | SGSGSGLGLN |
| 38 | SWIRM domain | CATGGAATGTAGGAACTATGTTGTTGCCAAATACATGGAGAATCC |
| | SNP (A/G | AGAAAAACGGTTCACTGTGTCTGATTTCGTGGGAGTGATACCTGGT |
| | causing K242R | CTTGAAACTG |
| | AA | |
| | substitution,; | |
| | position 728 bp | |
| | Abacus CDS) at | |
| | position 51 bp | |
| 39 | Zinc finger | TTGCTTCCATGATGGAAGGTATGTGATTGGTCACTCAAGCTTAGAT |
| | domain SNP | TTTATGAGGATGGATTCAACAAAAGATTATGGCGATCCAGATGGG |
| | (T/C causing | GAAAGTTGGA |
| | M398T AA | |
| | substitution;, | |
| | position 1193 bp | |
| | Abacus CDS) at | |
| | position 51 bp | |
| 40 | 21TP1B-21-1 | GAGTGGACAGCAGGCACGTATGCAAGATGTTTTTAAGATTCGAAA |
| | CAT2 3′UTR | TATGCAAGAAGGAACTAAATGGCGAAAAGTGCAGATTATAAATAT |
| | (1-500 bp) | GGAATGATGAATAACTCCTGTCTACTATCTTGTGTACTTTTCTTCTG |
| | | TAATCTTATGTTTTGTGTTGTTATATTATGTTATGTAACTCTTTATG |
| | | TAAACTATTTATTAAACTTAATAAATACTGGTCATGTGCACCAGTT |
| | | TCATATCCTTTGGTTTCCTTTGCTGAACTGTTCCCATTCAGCCAAAT |
| | | GAATGTGTCACTGTATTAATAGGGTTGATTATGCACCCCAAATCAA |
| | | ACTTTAATCCCTTTCATTATCTCAAGCAAGATTTGTTAGATAAAAT |
| | | GGTTCAAGGGTATATATCTGATTTCTGATATTAGTTATCATAACAA |
| | | CAGGTGATTGACTTAACATTTAACCTTTTGTGTTTGATATACAGAA |
| | | AGAGACTGGCCTTTGATTTGATTATGCTGATTGGTGCTT |
| 41 | 21TCV1-4-5 | GAGTGGACAGCAGGCACGTATGCAAGATGTTTTTAAGATTCGAAA |
| | CAT2 3′UTR (1 | TATGCAAGAAGGAACTAAATGGCGAAAAGTGCAGATTATAAATAT |
| | -495 bp) | GGAATGATGAATAACTCCTGTCTACTATCTTGTGTACTTTTCTTCTG |
| | | TAATCTTATGTTTTGTGTTGTTATATTATGTTATGTAACTCTTTATG |
| | | TAAACTATTTATTAAACTTAATAAATACTGGTCATGTGCACCAGTT |
| | | TCATATCCTTTGGTTTCCTTTGCTGAACTGTTCCCATTCAGCCAAAT |
| | | GAATGTGTCACTGTATTAATAGGGTTGATTATGCACCCCAAATCAA |
| | | ACTTTAATCCCTTTCATTATCTCAAGCAAGATTTGTTAGATAAAAT |
| | | GGTTCAAGGGTATATATCTGATTTCTGATATTAGTTATCATAACAA |
| | | CAGGTGATTGACTTAACATTTAACCTTTTGTGTTTGATATACAGAA |
| | | AGAGACTGGCCTTTGATTTGATTATGCTGATTGG |
| 42 | 21LCV1-1-13 | GAGTGGACAGCAGGCACGTATGCAAGATGTTTTTAAGATTCGAAA |
| | CAT2 3′UTR (1 | TATGCAAGAAGGAACTAAATGGCGAAAAGTGCAGATTATAAATAT |
| | -488 bp) | GGAATGATGAATAACTCCTGTGTACTATCTTGTGTACTTTTCTTCTG |
| | | TAATCTTATGTTTTGTGTTGTTATATTATGTTATGTAACTCTTTATG |
| | | TACACTATTTATTAAACTTAATAAATACTGGTCATGTGCACCAGTT |
| | | TCATATCCTTTGGTTTCCTTTGCTGAACTGTTCCCATTCAGCCAAAT |
| | | GAATGTGTCACTGTATTAATAGGGTAGATTATGCACCCCAAATCAA |
| | | ACTTTAATCCCTTTTATTATCTCAAGCAAGATTTGTTAGATAAAAT |
| | | GGTTCAAGGGTATATATCTGATATTAGTTATCATAACAACAGGTGA |
| | | TTGACTTAACATTTAACCTTTTGTGTTTGATATACAGAAAGAGACT |
| | | GGCCTTTGATTTGATTATGCTGATTGG |
| 43 | 21TP1B-1-1 | GAGTGGACAGCAGGCACGTATGCAAGATGTTTTTAAGATACGAAC |
| | CAT2 3′UTR (1 | TATGCAAGAAGGAACTAAATGGCGAAAAGTGCAGATTATAAATAT |
| | -489 bp) | GGAATGATGAATAACTCCTGTGTACTATCTTGTGTACTTTTCTTCTG |
| | | TAATCTTATGTTTTGTGTTGTTATATTATGTTATGTAACTCTTTGTG |
| | | TACACTATTTATTAAACTTAATAAATACTGGTCATGTGCACCAGTT |
| | | TCATATCCTTTGGTTTCCTTTGCTGAACTGTTCCCATTCAGCCAAAT |
| | | GAATGTGTCAATGTATTAATAGGGTTGATTATGCACCCCAAATCAA |
| | | ACTTAAATCCCTTTTATTATCTCAAGCAAGATTTGTTAGATCAAAT |
| | | GGTTCAAGGGTATCTGATATTAGTTATCATAACACCAGGTGATTGA |
| | | CTTAACATTTAACCTTTTGTGTTTAATATACAGAAAGAGACTGGCC |
| | | TTTGATTTGATTATGCTGATTGGTGCTT |
| 44 | Abacus CAT2 | GAGTGGACAGCAGGCACGTATGCAAGATGTTTTTAAGATTCGAAA |
| | 3′UTR (1-493 | TATGCAAGAAGGAACTAAATGGCGAAAAGTGCAGATTATAAATAT |
| | bp) | GGAATGATGAATAACTCCTGTGTACTATCTTGTGTACTTTTCTTCTG |
| | | TAATCTTATGTTTTGTGTTGTTATATTATGTTATGTAACTCTTTATG |
| | | TACACTATTTATTAAACTTAATAAATACTGGTCATGTGCACCAGTT |
| | | TCATATCCTTTGGTTTCCTTTGCTGAACTGTTCCCATTCAGCCAAAT |
| | | GAATGTGTCACTGTATTAATAGGGTAGATTATGCACCCCAAATCAA |
| | | ACTTTAATCCCTTTTATTATCTCAAGCAAGATTTGTTAGATAAAAT |
| | | GGTTCAAGGGTATATATCTGATATTAGTTATCATAACAACAGGTGA |
| | | TTGACTTAACATTTAACCTTTTGTGTTTGATATACAGAAAGAGACT |
| | | GGCCTTTGATTTGATTATGCTGATTGGTGCTT |
| 45 | CAT2 3′UTR | CTGTATTAATAGGGTAGATTATGCACCCCAAATCAAACTTTAATCC |
| | SNP (T/C; | CTTTTATTATCTCAAGCAAGATTTGTTAGATAAAATGGTTCAAGGG |
| | position 338 bp | TATATATCT |
| | Abacus 3′UTR) | |
| | at position 51 bp | |
| 46 | CAT2 3′UTR | CTCAAGCAAGATTTGTTAGATAAAATGGTTCAAGGGTATATATCTG |
| | indel (as shown | ATATAGTTATCATAACAACAGGTGATTGACTTAACATTTAACCTTT |
| | here, the | TGTGTTT |
| | insertion of | |
| | CTGATAT is | |
| | absent in the | |
| | Abacus | |
| | reference | |
| | genome between | |
| 47 | position | GTAGTTATGGACCGGATCCGCAGAATTAGTATATCTCACAGTTAA |
| | 20TP1B-1020-1 | |
| | ACS12 CDS | |
| | (1474-1518 bp) | |
| 48 | 20TP1B-1016-6 | GTAGTTATGGACCGGATCCGCAGAATTACCGAAACTAGTATATCTC |
| | ACS12 CDS | GCAGTTAA |
| | (1474-1527 bp) | |
| 49 | 20TP1B-1015-5 | GTAGTTATGGACCGGATCTGCAGAATTACCGAAAGTAGTATATCTC |
| | ACS12 CDS | ACAGTTAA |
| | (1474-1527 bp) | |
| 50 | Abacus ACS12 | ATGCGCCTCATTGTGCCACTCCAAGGTGTGGTGCAAGGTAGAGGC |
| | CDS (1-1518 | GGCCTTATTTGGGGCTCTCTTATCCCCTGCGCCCTCTTCTACTTCCT |
| | bp) | CCAGTTCTACCTCAAACGAAACCGTCCTCCAAGTACCTCCTCGAAT |
| | | CCTCCGTCGCCATCTCAGTCTTCCTCCAATCTTCTCGAACTCCAAA |
| | | GGTCGTCTTCCAGGTCAAACTTGTCCAGTCGTGGATCCATAGGCCG |
| | | AGTCACACTATCCACCCGGGGAAGTTTGATTGCAAAACCAAATGA |
| | | CTCCCCTTACTATATTGGGCTGGATAAGGTTAGGGAGGATCCTTAT |
| | | GACAGATTGGGTAACCCAAATGGGGTAATTGAGCTCGGTTTGGCC |
| | | GAGAACAAGTTGAGTTTTGATCTTATTGAGAAATGGGTTTCTGGGA |
| | | ATTGGACTAATTCGATAATTGGAACCAATGGTGGCGATTTGAATAT |
| | | TAGCAGCATTGCAGCATACCAACCGTTCGATGGATTGACTGAACT |
| | | GAAAGTGGCTATTGCTGATTTCATGACTCGGGTCGTGGAAAGAAC |
| | | TATCACATTTGACCCGTCTCAAATGGTGTTAACTGCTGGAGCAACC |
| | | CCTGCTACTGAAATTTTAAGCTTTTGCTTAGCAGACAATGGAAACG |
| | | CATTTCTTGTTCCCACACCTTATTATCCCGGCTTCGATAGAGACATT |
| | | AGATGGCGAGCTGGGGTGGAGCTAATTCCTGTTCATTGCCGTAGC |
| | | GCTGATAACTTTGCTTTGAACATTACTGTGCTTGATCAAGCATTTA |
| | | ATCAGGCAAGAAAGCGAGGAGTAAAAGTTCGTGGAATTCTAATTT |
| | | CAAATCCTGTTAATCCTGTTGGCAATGTACTGTCTCGGGAAACACT |
| | | TTGTGGTATTTTAGACTTTGCCCAGGAGAAGAACATCCATGTTATA |
| | | TCTGATGAAATATTTGCTGGGTCCATGTATGGAAATGAGGAGTTTG |
| | | TGAGCATGGCAGAAATTATTGAGACAGAGAATTTTGAAAAGAGCA |
| | | GGGTTCACATAATATACGGACTGTCGAAAGACCTATCTATTCCGGG |
| | | CTTTAGAGTTGGTGTTATTTATTCTTACAACGAGAATGTTTTGGCC |
| | | GCTGCAAAAAAGTTAACAAGATTTTCTTCAATCTCTGCTCCGGCCC |
| | | AGCGTCTTCTAGTATCGATGCTTTTAGATACGAGATTCCTTGAGGA |
| | | TTATATGAAGATAAACAAAGAAAGGATACGTAATATGCATAACTT |
| | | ATTTGTAGCAGGTTTGGGGCAATTAGGTATCAAGTGCGCAAAGAG |
| | | TAGTGCTGGTTTCTACTGTTGGGCAAATATGAGTGGGTTTATTCCA |
| | | TCATACAGTGAGAAAGGGGAACTTGAGTTATGGGAAAAGCTGTTG |
| | | AATGTTTCTAAGATCAATGTAACTCCTGGTTCAGCTTGCCACTGCA |
| | | TAGAGCCGGGCTGGTTTCGTTGTTGTTTTACTACTATAGCTGAAGA |
| | | TGATATCCCTGTAGTTATGGACCGGATCCGCAGAATTAGTATATCT |
| | | CACAGTTAA |
| 51 | 20TP1B-1020-1 | VVMDRIRRISISHS |
| | ACS12 protein | |
| | sequence (492- | |
| | 505 AA) | |
| 52 | 20TP1B-1016-6 | VVMDRIRRITETSISRS |
| | ACS12 protein | |
| | sequence (492- | |
| | 508 AA) | |
| 53 | 20TP1B-1015-5 | VVMDRICRITESSISHS |
| | ACS12 protein | |
| | sequence (492- | |
| | 508 AA) | |
| 54 | Abacus ACS12 | MRLIVPLQGVVQGRGGLIWGSLIPCALFYFLQFYLKRNRPPSTSSNPPS |
| | protein sequence | PSQSSSNLLELQRSSSRSNLSSRGSIGRVTLSTRGSLIAKPNDSPYYIGL |
| | (1-505 AA) | DKVREDPYDRLGNPNGVIELGLAENKLSFDLIEKWVSGNWTNSIIGTN |
| | | GGDLNISSIAAYQPFDGLTELKVAIADFMTRVVERTITFDPSQMVLTA |
| | | GATPATEILSFCLADNGNAFLVPTPYYPGFDRDIRWRAGVELIPVHCR |
| | | SADNFALNITVLDQAFNQARKRGVKVRGILISNPVNPVGNVLSRETLC |
| | | GILDFAQEKNIHVISDEIFAGSMYGNEEFVSMAEIIETENFEKSRVHIIY |
| | | GLSKDLSIPGFRVGVIYSYNENVLAAAKKLTRESSISAPAQRLLVSMLL |
| | | DTRFLEDYMKINKERIRNMHNLFVAGLGQLGIKCAKSSAGFYCWAN |
| | | MSGFIPSYSEKGELELWEKLLNVSKINVTPGSACHCIEPGWFRCCFTTI |
| | | AEDDIPVVMDRIRRISISHS |
| 55 | 20TP1B-1020-1 | ATTATTTGTTCATTTATTGTTTTATTTTTTCTTTCTTCTTCTCAATCA |
| | ACS12 3′UTR | CCTTGTTTTACAATTTTGAACTCTTTTGGGCTTTCAGCCTTAGAAGT |
| | (1-199 bp) | AAGGGATTTAGATATAAAGTTTTCTGGTGAAGTTTCATATAATATC |
| | | ATTATCAGCTAAAATCTCTTTTACAATGCACTTTTTCAATTAATTTA |
| | | GGATAAGAAAA |
| 56 | 20TP1B-1016-6 | ATTATTTGTTCATTTATTGTTTTATTTTTTCTTCTTCTCAATCACCTT |
| | ACS12 3′UTR | GTTTTACAATTTTGAACTCTTTTGGGCTTTCAGCCTTAGAAGTAAG |
| | (1-195 bp) | GGATTTAGATATAAAGTTTTCTGGTGAAGTTTCATATAATATCATT |
| | | ATCAGCTAAAATCTCTTTTACAATGCACTTTTTCAATTAATTTAGG |
| | | ATAAGAAAA |
| 57 | 20TP1B-1015-5 | ATTATTTGTTCATTTATTGTTTTATTTTTTCTTCTTCTCAATCACCTT |
| | ACS12 3′UTR | GTTTTACAATTTTGAACTCTTTTGGGCTTTCAGCCTTAGAAGTAAG |
| | (1-195 bp) | GGATTTAGATATAAAGTTTTTTGGTGAAGTTTCATATAATATCATT |
| | | ATCAGCTAAAATCTCTTTTACAATGCACTTTTTCAATTAATTTAGG |
| | | ATAAGAAAA |
| 58 | Abacus ACS12 | ATTATTTGTTCATTTATTGTTTTATTTTTTCTTTCTTCTTCTCAATCA |
| | 3′UTR (1-199 | CCTTGTTTTACAATTTTGAACTCTTTTGGGCTTTCAGCCTTAGAAGT |
| | bp) | AAGGGATTTAGATATAAAGTTTTCTGGTGAAGTTTCATATAATATC |
| | | ATTATCAGCTAAAATCTCTTTTACAATGCACTTTTTCAATTAATTTA |
| | | GGATAAGAAAA |
| 59 | ACS129 | ACTATAGCTGAAGATGATATCCCTGTAGTTATGGACCGGATCCGCA |
| | nucleotide indel | GAATTAGTATATCTCACAGTTAAATTATTTGTTCATTTATTGTTTTA |
| | (as shown here, | TTTTTTC |
| | the insertion of | |
| | TACCGAAAS* | |
| | * is absent in the | |
| | Abacus | |
| | reference | |
| | genome between | |
| | positions 1499- | |
| | 1500 bp Abacus | |
| | CDS) after | |
| | position 50 bp | |
| 60 | ACS12 3′UTR 4 | GCAGAATTAGTATATCTCACAGTTAAATTATTTGTTCATTTATTGTT |
| | nucleotide indel | TTATTTTTTCTTTCTTCTTCTCAATCACCTTGTTTTACAATTTTGAAC |
| | (TTTT) between | TCTTTTGGG |
| | positions 25-28 | |
| | bp of the | |
| | Abacus 3′UTR) | |
| | at positions 51- | |
| | 54 bp | |
| 61 | ACS12 3′UTR | TTAGTATATCTCACAGTTAAATTATTTGTTCATTTATTGTTTTATTTT |
| | C/T substitution | TTCTTTCTTCTTCTCAATCACCTTGTTTTACAATTTTGAACTCTTTTG |
| | (at position 31 | GGCTT |
| | bp of the | |
| | Abacus 3′UTR) | |
| | at position 51 bp | |

It will be apparent that the precise details of the methods or compositions described may be varied or modified without departing from the spirit of the described aspects of the disclosure. We claim all such modifications and variations that fall within the scope and spirit of the claims below.

1. A method for selecting a cannabis plant having resistance to hermaphroditism, comprising:
(i) obtaining a nucleic acid sample from the cannabis plant or its germplasm;
(ii) detecting one or more genetic markers in the nucleic acid sample that indicate resistance to hermaphroditism, wherein the one or more genetic markers comprise a polymorphism at one or more of nucleotide positions:
(a) 5,705,332 on chromosome X;
(b) 5,732,323 on chromosome X;
(c) 5,747,057 on chromosome X;
(d) 5,877,981 on chromosome X;
(e) 5,920,712 on chromosome X,
(f) 6,053,325 on chromosome X,
(g) 6,181,263 on chromosome X,
(h) 6,186,518 on chromosome X,
(i) 6,192,534 on chromosome X,
(j) 6,261,819 on chromosome X,
(k) 6,285,113 on chromosome X,
(l) 6,695,193 on chromosome X,
(m) 6,961,002 on chromosome X,
(n) 17,971,672 on chromosome X,
(o) 14,810,444 on chromosome 3,
(p) 57,051,092 on chromosome 3,
(q) 74,435,555 on chromosome 3,
(r) 5,233,698 on chromosome 4, and(s) 12,961,444 on chromosome 4,
according to the Abacus Cannabis reference genome version Csat_AbacusV2, NCBI assembly accession GCA_025232715.1;
(iii) indicating resistance to hermaphroditism, and
(iv) selecting the cannabis plant indicating resistance to hermaphroditism.

2. The method of claim 1, wherein the one or more genetic markers comprise a polymorphism at:
nucleotide position 5,732,323 on chromosome X, nucleotide position 17,971,672 on chromosome X, and nucleotide position 14,810,444 on chromosome 3.

3. The method of claim 1, wherein the one or more genetic markers comprise a polymorphism at nucleotide position 5,732,323 on chromosome X, nucleotide position 17,971,672 on chromosome X, or nucleotide position 14,810,444 on chromosome 3.

4. The method of claim 1, wherein the nucleotide position comprises:
(1) an A/A or G/A genotype at position 5,705,332 on chromosome X;
(2) an A/A or G/A genotype at position 5,732,323 on chromosome X;
(3) a G/G or A/G genotype at position 5,747,057 on chromosome X;
(4) a T/T C/T genotype at position 5,877,981 on chromosome X;
(5) a C/C or A/C genotype at position 5,920,712 on chromosome X;
(6) a T/T or C/T genotype at position 6,053,325 on chromosome X;
(7) a T/T or C/T genotype at position 6,181,263 on chromosome X;
(8) an A/A or G/A genotype at position 6,186,518 on chromosome X;
(9) a C/C or A/C genotype at position 6,192,534 on chromosome X;
(10) an A/A or G/A genotype at position 6,261,819 on chromosome X;
(11) an A/A or G/A genotype at position 6,285,113 on chromosome X;
(12) an A/A or C/A genotype at position 6,695,193 on chromosome X;
(13) a T/T or C/T genotype at position 6,961,002 on chromosome X;
(14) a T/T or C/T genotype at position 17,971,672 on chromosome X;
(15) a T/T or C/T genotype at position 14,810,444 on chromosome 3;
(16) a G/G or G/A genotype at position 57,051,092 on chromosome 3;
(17) a C/C genotype at position 74,435,555 on chromosome 3;
(18) a G/G or G/A genotype at position 5,233,698 on chromosome 4; or
(19) a G/G genotype at position 12,961,444 on chromosome 4.

5. The method of claim 4, wherein the nucleotide position comprises:
the A/A or G/A genotype at position 5,732,323 on chromosome X; the T/T or C/T genotype at position 17,971,672 on chromosome X; or the T/T or C/T genotype at position 14,810,444 on chromosome 3; or
the A/A or G/A genotype at position 5,732,323 on chromosome X; the T/T or C/T genotype at position 17,971,672 on chromosome X; and the T/T or C/T genotype at position 14,810,444 on chromosome 3.

6. A method for selecting a cannabis plant having resistance to hermaphroditism, comprising:
(i) obtaining a nucleic acid sample from the cannabis plant or its germplasm;
(ii) detecting one or more genetic markers in the nucleic acid sample that indicate resistance to hermaphroditism, wherein the one or more genetic markers comprise a polymorphism within a haplotype, and wherein the haplotype comprises the region:
(1) between positions 5,696,400 and 5,714,336 on chromosome X;
(2) between positions 5,725,231 and 5,737,968 on chromosome X;
(3) between positions 5,737,968 and 5,750,301 on chromosome X;
(4) between positions 5,860,363 and 5,894,065 on chromosome X;
(5) between positions 5,894,065 and 5,929,330 on chromosome X;
(6) between positions 6,032,408 and 6,059,371 on chromosome X;
(7) between positions 6,133,760 and 6,189,246 on chromosome X;
(8) between positions 6,189,246 and 6,197,726 on chromosome X;
(9) between positions 6,258,772 and 6,290,824 on chromosome X;
(10) between positions 6,688,670 and 6,715,018 on chromosome X;
(11) between positions 6,932,849 and 6,970,023 on chromosome X;
(12) between positions 17,900,648 and 17,980,443 on chromosome X;
(13) between positions 14,797,288 and 14,824,861 on chromosome 3;
(14) between positions 56,616,473 and 57,090,960 on chromosome 3;
(15) between positions 74,422,393 and 74,437,841 on chromosome 3;
(16) between positions 5,216,694 and 5,243,017 on chromosome 4; or
(17) between positions 12,923,613 and 13,009,666 on chromosome 4;
according to the Abacus Cannabis reference genome version Csat_AbacusV2, NCBI assembly accession GCA_025232715.1;
(iii) indicating resistance to hermaphroditism; and
(iv) selecting the cannabis plant indicating resistance to hermaphroditism.

7. The method of claim 1, wherein the detecting comprises using an oligonucleotide primer set or probe.

8. The method of claim 1, wherein the detecting comprises PCR, quantitative PCR (qPCR), and/or sequencing.

9. The method of claim 1, further comprising crossing the selected plant comprising the indicated resistance to hermaphroditism to produce one or more F1 or additional progeny plants, wherein at least one of the F1 or additional progeny plants comprises the indicated resistance to hermaphroditism.

10. The method of claim 9, wherein the crossing comprises selfing, sibling crossing, outcrossing, or backcrossing.

11. (canceled)

12. The method of claim 10, wherein the selfing, sibling crossing, outcrossing, or backcrossing comprises marker-assisted selection.

13-15. (canceled)

16. A method of identifying a cannabis plant having resistance to hermaphroditism, comprising:
(i) obtaining a nucleic acid sample from the plant or its germplasm;
(ii) analyzing the sample to detect one or more nucleic acid polymorphisms in:
(a) switch/sucrose nonfermenting 3C (SWI3C),
(b) catalase 2 (CAT2), or
(c) 1-aminocyclopropane-1-carboxylate synthase 12 (ACS12),
wherein the one or more nucleic acid polymorphisms are associated with resistance to hermaphroditism, thereby identifying a cannabis plant having resistance to hermaphroditism.

17. The method of claim 16, wherein the one or more nucleic acid polymorphisms are detected in SWI3C and comprise:
(a) a substitution at position 728 bp from the start codon of the coding sequence of Abacus SWI3C (SEQ ID NO: 33), or
(b) a substitution at position 1193 bp from the start codon of in the coding sequence of Abacus SWI3C (SEQ ID NO: 33).

18. (canceled)

19. The method of claim 16, wherein the one or more nucleic acid polymorphisms are detected in CAT2 and comprise:
(a) a substitution at position 338 bp from the start of the 3′UTR of Abacus CAT2 (SEQ ID NO: 44), or
(b) an insertion starting at 394 bp from the start of the 3′UTR of Abacus CAT2 (SEQ ID NO: 44).

20. (canceled)

21. The method of claim 16, wherein the one or more nucleic acid polymorphisms are detected in ACS12 and comprise:
(a) a deletion at position 1500 bp from the start codon of the coding sequence of Abacus ACS12 (SEQ ID NO. 50), or
(b) an insertion at position 25 bp from the start of the 3′UTR of Abacus ACS12 (SEQ ID NO: 58) or,
(c) a substitution at position 31 bp from the start of the 3′UTR of Abacus ACS12 (SEQ ID NO: 58).

22. (canceled)

23. The method of claim 16, wherein analyzing the sample comprises analyzing at least two of SWI3C, CAT2, and ACS12.

24-26. (canceled)

27. A plant identified by the method of claim 16.

28. A method of plant breeding, comprising crossing the plant identified by the method of claim 16.

29. (canceled)

30. A method of producing a genetically engineered cannabis plant resistant to hermaphroditism, comprising:
introducing a genetic modification in switch/sucrose nonfermenting 3C (SWI3C), catalase 2 (CAT2), or 1-aminocyclopropane-1-carboxylate synthase 12 (ACS12) that is associated with resistance to hermaphroditism; or
introducing a beneficial allele of SWI3C, CAT2, or ACS12.

31-35. (canceled)

36. A plant produced by the method of claim 30.

37. (canceled)
